(19) United States
(12) Patent Application Publication
(10) Pub. No.: US 2010/0137305 A1
(43) Pub. Date: Jun. 3, 2010

(54) AMINOPYRIMIDINES USEFUL AS KINASE INHIBITORS

(75) Inventors: Hayley Binch, Encinitas, CA (US); Stephen Young, Oxford (GB); Christopher Davis, Salisbury (GB); Michael Mortimore, Burford (GB); John Studley, Abingdon (GB); Daniel Robinson, Abingdon (GB); Michael O'Donnell, Abingdon (GB); Dean Boyall, Faringdon (GB); Joanne Pinder, Abingdon (GB); Simon Everitt, Beaconsfields (GB); Julian Golec, Farringdon (GB)

Correspondence Address: VERTEX PHARMACEUTICALS INC., 130 WAVERLY STREET, CAMBRIDGE, MA, 02139-4242, US

(73) Assignee: VERTEX PHARMACEUTICALS INCORPORATED, Cambridge, MA (US)

(21) Appl. No.: 12/562,181

(22) Filed: Sep. 18, 2009

Related U.S. Application Data
(63) Continuation of application No. PCT/US2008/057465, filed on Sep. 18, 2008.

(51) Int. Cl. A61K 31/5377; A61K 31/506; C07D 413/14; A61P 35/00; C07D 403/14; A61P 35/02

(52) U.S. Cl. 514/234.5; 514/235.8; 514/274; 544/122; 544/230; 544/317

(57) ABSTRACT

The present invention relates to compounds useful as inhibitors of Aurora protein kinases. The invention also provides pharmaceutically acceptable compositions comprising those compounds and methods of using the compounds and compositions in the treatment of various disease, conditions, and disorders. The invention also provides processes for preparing compounds of the invention.

### TECHNICAL FIELD OF THE INVENTION

The present invention relates to compounds useful as inhibitors of Aurora protein kinases. The invention also relates to pharmaceutically acceptable compositions comprising the compounds of the invention, methods of using the compounds and compositions in the treatment of various disorders, and processes for preparing the compounds.

### BACKGROUND OF THE INVENTION

The Aurora proteins are a family of three related serine/threonine kinases (termed Aurora-A, -B and -C) that are essential for progression through the mitotic phase of cell cycle. Specifically Aurora-A plays a crucial role in centrosome maturation and segregation, formation of the mitotic spindle and faithful segregation of chromosomes. Aurora-B is a chromosomal passenger protein that plays a central role in regulating the alignment of chromosomes on the meta-phase plate, the spindle assembly checkpoint and for the correct completion of cytokinesis.

Overexpression of Aurora-A, -B or -C has been observed in a range of human cancers including colorectal, ovarian, gastric and invasive duct adenocarcinomas.

A number of studies have now demonstrated that depletion or inhibition of Aurora-A or -B in human cancer cell lines by siRNA, dominant negative antibodies or neutralizing antibodies disrupts progression through mitosis with accumulation of cells with 4N DNA, and in some cases this is followed by endoreduplication and cell death.

The Aurora kinases are attractive targets due to their association with numerous human cancers and the roles they play in the proliferation of these cancer cells. It would be desirable to have an Aurora kinase inhibitor with favorable drug-like properties, such as stability in human liver microsomes. Accordingly, there is a need for compounds that inhibit Aurora kinases and also exhibit favorable drug-like properties.

### SUMMARY OF THE INVENTION

This invention provides compounds and pharmaceutically acceptable compositions thereof that are useful as inhibitors of Aurora protein kinases. More specifically, this invention provides compounds that are metabolically stable in human liver microsomes and/or potently inhibit cell proliferation.

These compounds are represented by formula I:

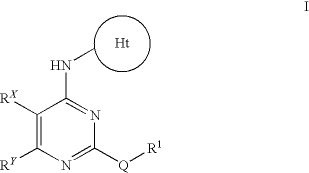

or a pharmaceutically acceptable salt thereof, wherein the variables are as defined herein.

These compounds and pharmaceutically acceptable compositions thereof are useful for inhibiting kinases in vitro, in vivo, and ex vivo. Such uses include treating or preventing myeloproliferative disorders and proliferative disorders such as melanoma, myeloma, leukemia, lymphoma, neuroblastoma, and cancer. Other uses include the study of kinases in biological and pathological phenomena; the study of intracellular signal transduction pathways mediated by such kinases; and the comparative evaluation of new kinase inhibitors.

### DETAILED DESCRIPTION OF THE INVENTION

One embodiment of this invention provides a compound of formula I:

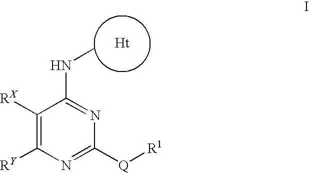

or a pharmaceutically acceptable salt thereof, wherein:
Ht is

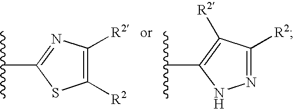

R² is H, C₁₋₃ alkyl, or cyclopropyl;
R^{2′} is H;
Q is —O—, —S—, or —C(R′)₂—;
R^{X} is H or F;
R^{Y} is

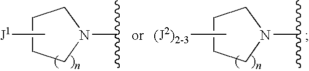

J¹ is F, NR⁴R⁵, CN, OR⁶, oxo (═O), or C₂₋₆alkyl optionally substituted with 1 occurrence of OH or OCH₃;
each J² is independently C₁₋₆alkyl, F, NR⁴R⁵, CN, or OR⁶; or two J² groups, together with the atom(s) to which they are bound, form a 4-7 membered heterocyclyl ring containing 1-2 heteroatoms selected from N or O; wherein said ring is optionally substituted with 0-3 J^{R};
n is 1 or 2;
R⁴ is H, C₁₋₅alkyl, or C₃₋₆ cycloalkyl;
R⁵ is C₁₋₅alkyl or C₃₋₆ cycloalkyl;
or R⁴ and R⁵, together with the nitrogen atom to which they are bound, form a 3-6 membered monocyclic ring containing 1-2 heteroatoms selected from O, N, or S; wherein said monocyclic ring is optionally substituted with 0-3 J^{R};
R⁶ is H, C₁₋₄alkyl or C₃₋₆ cycloalkyl; wherein said C₁₋₄alkyl or C₃₋₆ cycloalkyl is optionally substituted with 1-3 fluorine atoms;
J^{R} is F or R⁷;
R¹ is phenyl or a 6-membered heteroaryl ring, wherein said heteroaryl has 1-4 ring heteroatoms selected from O, N, and S; R¹ is optionally substituted with 0-4 occurrences of —NHC(O)R³ or 0-4 fluorine atoms;
R³ is C₁₋₆aliphatic or phenyl, wherein said R³ is optionally substituted with 0-6 J³;
each J³ is independently halo, C₁₋₆alkyl, —O—(C₁₋₆alkyl), —S—(C₁₋₆ alkyl), nitro, or CN, wherein said C₁₋₆alkyl group is optionally substituted with 0-3 flourine atoms; or two J³ groups, together with the carbon atom to which they are bound, form a 3-5 membered monocyclic group containing 0-1 heteroatom selected from O, N, and S;
each R⁷ is independently C₁₋₆ aliphatic; a 5-6 membered heteroaryl containing 1-4 heteroatoms selected from O, N, or S; each R⁷ is optionally substituted with 0-3 J⁷; and
J⁷ is independently NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄aliphatic), or haloC₁₋₄aliphatic.

For the avoidance of doubt, it should be understood that in a compound of this invention, if Rx is H, Ht is

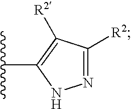

R² is cyclopropyl, R^{2′} is H, Q is S, R¹ is phenyl, and R³ is ethyl; then R^{Y} is not number

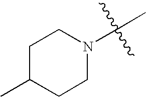

(4-methylpiperidine).

For the avoidance of doubt, it should also be understood that when R^{X} is H, Ht is

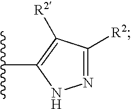

R² is methyl, R^{2′} is H, Q is S, R¹ is phenyl, and R³ is ethyl; then R¹ is not

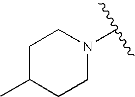

(4-methylpiperidine).

One embodiment of this invention provides a compound of formula I or a pharmaceutically acceptable salt thereof, wherein the variables are as defined herein:
J¹ is F, NR⁴R⁵, CN, OR⁶, or oxo (═O), optionally substituted with 1 occurrence of OH or OCH₃;
each J² is independently F, NR⁴R⁵, CN, or OR⁶, or two J² groups, together with the atom(s) to which they are bound, form a 4-7 membered heterocyclyl ring containing 1-2 heteroatoms selected from N or O, wherein said ring is optionally substituted with 0-3 J^{R};
n is 1 or 2; and
the values of the remaining variables are as described in formula I above.
In some embodiments, n is 1.

Another embodiment provides a compound of formula II:

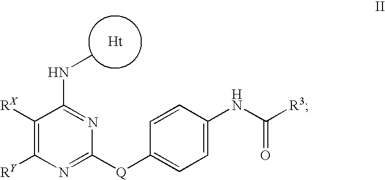

wherein the variables are as defined herein.

In some embodiments, Q is S.

In other embodiments, Rx is H.

In some embodiments, R² is H or optionally substituted C₁₋₆ aliphatic. In other embodiments, R² is C₁₋₃ alkyl or cyclopropyl. In some embodiments, R² is C₁₋₃ alkyl.

In some embodiments, R^{2′} is H. In other embodiments, R^{2′} is H and R² is C₁₋₃ alkyl or cyclopropyl.

In another embodiment, R¹ is phenyl. In some of these embodiments, R¹ is substituted at the para position. In some embodiments, R¹ is optionally substituted with 1 occurrence of —NHC(O)R³. In some embodiments, R¹ is

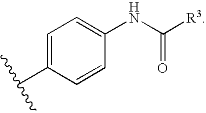

In some embodiments, R³ is C₁₋₆aliphatic wherein said R³ is optionally substituted with 0-6 J³. In some embodiments, R³is —CH₂CH₃, CH₂CF₃, CH₂CH₂CF₃, cyclopropyl, or

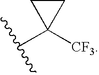

In other embodiments, R³ is phenyl. In some of these embodiments, R³ is substituted in the ortho position with J³. In some embodiments, J³ is halogen, CF₃, C₁₋₃alkyl, —S—(C₁₋₃alkyl), or OCF₃.

In some embodiments, Ht is

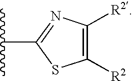

In other embodiments, Ht is

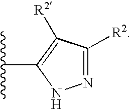

In some embodiments, n is 1. In other embodiments, n is 2.

In some embodiments, J² is independently C₁₋₆alkyl, F, NR⁴R⁵, CN, OR⁶, or R⁷.

In some embodiments, J¹ is F. In other embodiments, J¹ is NR⁴R⁵.

In some embodiments, R⁴ and R⁵, together with the nitrogen atom to which they are bound, form a 5-6 membered monocyclic ring containing 1-2 heteroatoms selected from O, N, or S; wherein said monocyclic ring is optionally substituted with 0-3 J^{R}.

In some embodiments, said monocyclic ring is a ring selected from piperidine, piperazine, morpholine, or pyrrolidine. In some embodiments, said piperidine, piperazine, morpholine, or pyrrolidine ring is optionally substituted with F or R⁷. In some embodiments, R⁷ is C₁₋₆aliphatic.

Another embodiment provides compounds wherein R^{Y} is

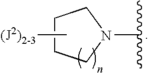

In some embodiments, n is 2. In some embodiments, R^{Y} is

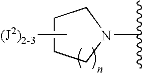

and n is 2.

In some embodiments, two J² groups, together with the atom(s) to which they are bound, form a 4-7 membered heterocyclyl ring containing 1-2 heteroatoms selected from N or O; wherein said ring is optionally substituted with 0-3 J^{R}.

In some embodiments, the two J² groups are attached to the same atom to form a spirocyclic compound. In some embodiments, said heterocyclyl ring contains 1 heteroatom. In some embodiments, said heteroatom is nitrogen. In some embodiments, said heterocyclyl ring is selected from piperidine or pyrrolidine. In some embodiments, said heterocyclyl ring is optionally substituted with 1 J^{R}.

In some embodiments, J^{R} is R⁷ and the R⁷ is C₁₋₆ aliphatic. In some embodiments, R⁷ is C₁₋₆alkyl. In some embodiments, R⁷ is methyl.

In some embodiments, R^{Y} is

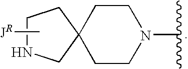

In other embodiments, R^{Y} is

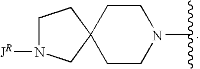

Another embodiment provides compounds wherein R^{Y} is

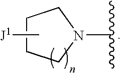

In some embodiments, n is 1. In some embodiments, R^{Y} is

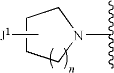

and n is 1.

In some embodiments, J¹ is F and R¹ is substituted with 1 occurrence of —NHC(O)R³. In some embodiments, R³ is C₁₋₆aliphatic, wherein said R³ is substituted with 0-6 J³;

each J³ is halo. In some embodiments, R³ is CH₂CF₃. In other embodiments, R³ is CH₂CH₂CF₃. In some embodiments, R³ is ethyl or cyclopropyl.

In some embodiments, R^{Y} is

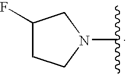

In other embodiments, R^{Y} is

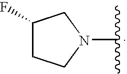

In some embodiments, R^{Y} is

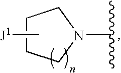

n is 1, J¹ is NR⁴R⁵, R¹ is substituted with 1 occurrence of —NHC(O)R³, and R³ is C₂₋₆aliphatic, wherein said R³ is substituted with 0-6 J³; each J³ is halo. In some embodiments, R^{Y} is

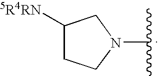

In some embodiments, R^{Y} is

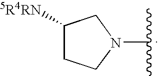

In some embodiments, R³ is CH₂CF₃ or CH₂CH₂CF₃. In other embodiments, R³ is CH₂CF₃. In some embodiments, R³ is ethyl or cyclopropyl.

In some embodiments, the variables of Formula I and Formula II include those shown in Table 1 below.

Another embodiment provides compounds selected from Table 1.

TABLE 1

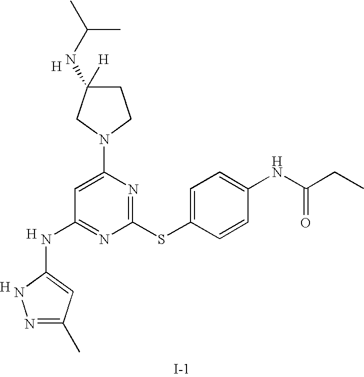

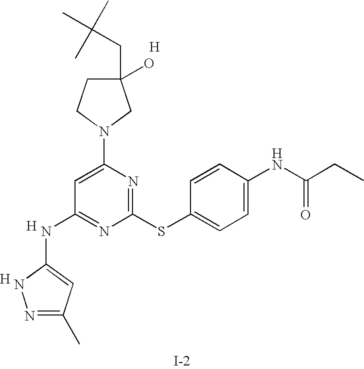

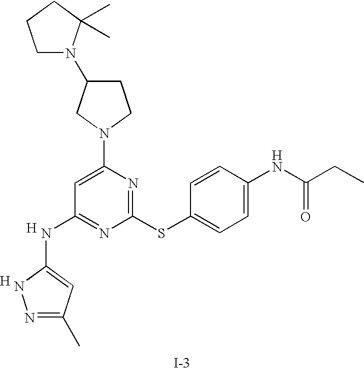

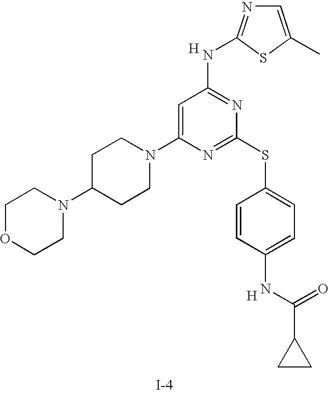

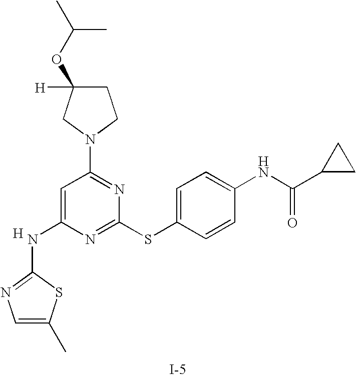

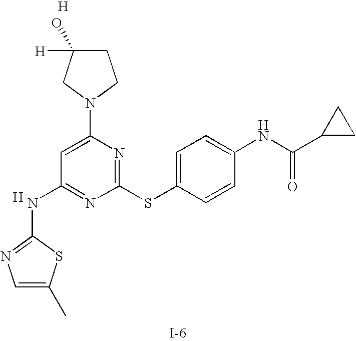

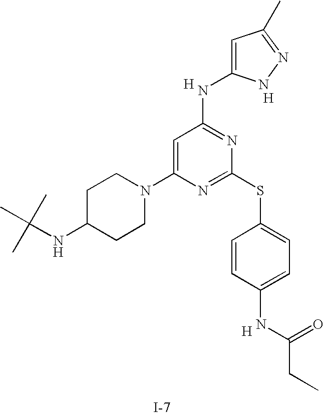

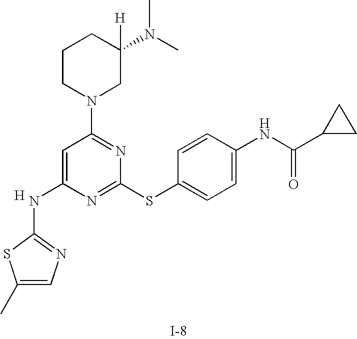

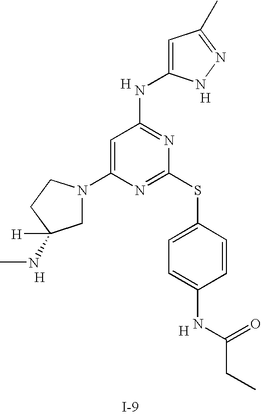

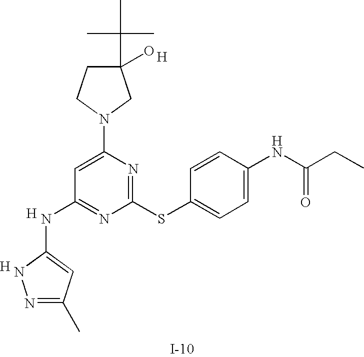

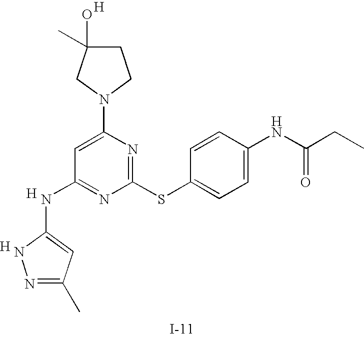

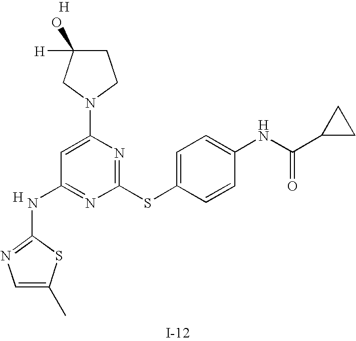

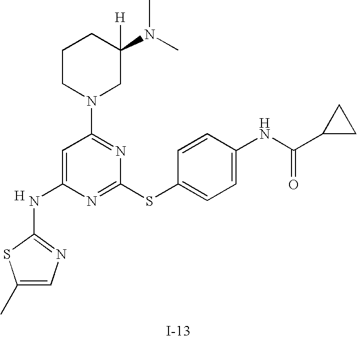

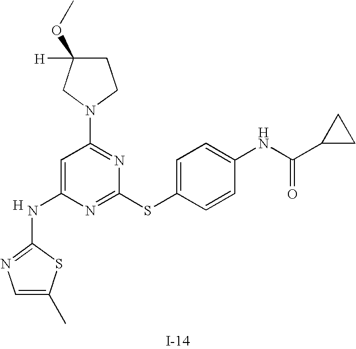

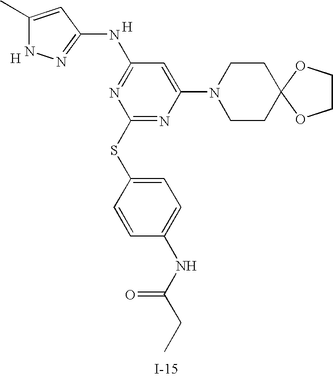

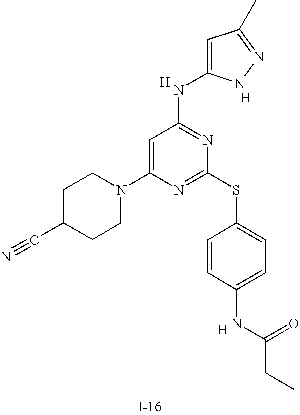

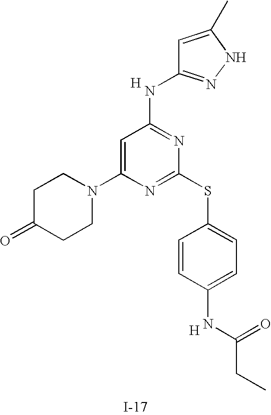

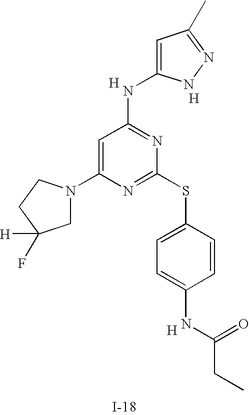

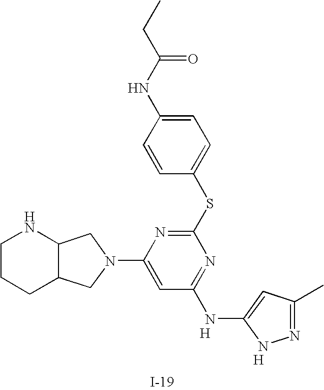

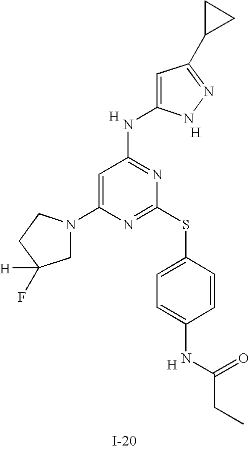

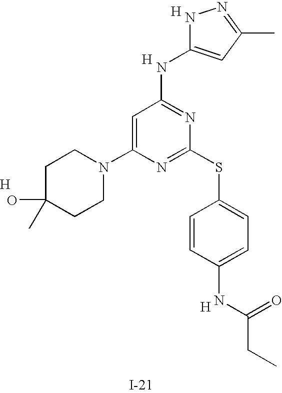

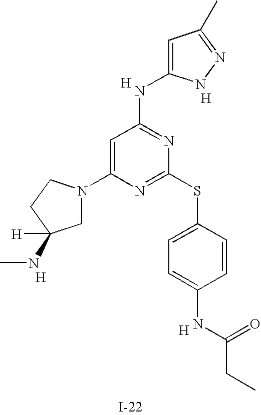

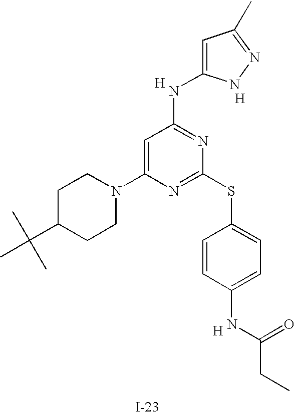

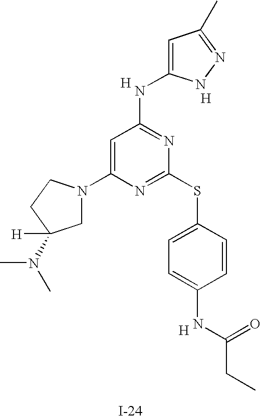

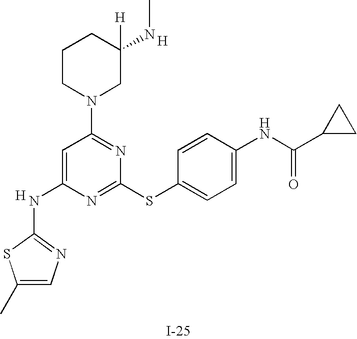

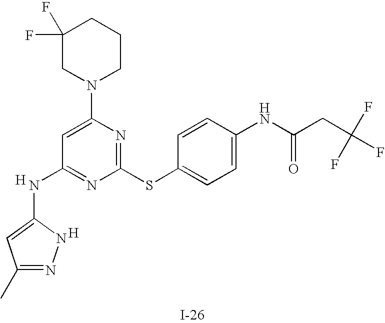

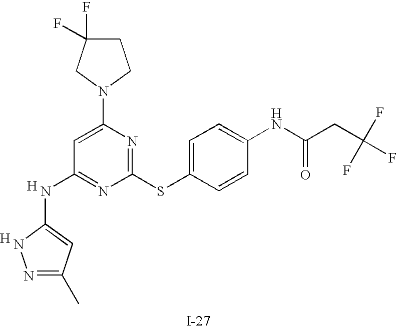

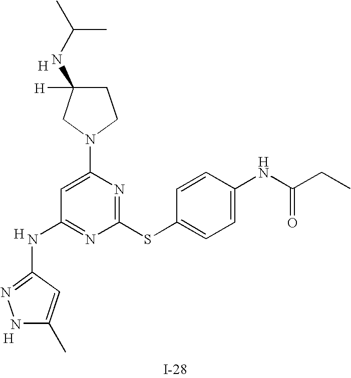

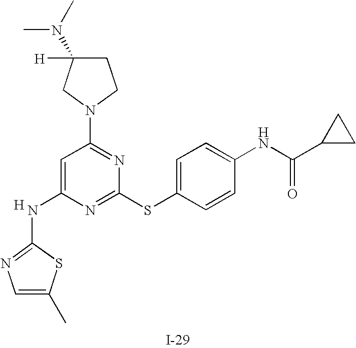

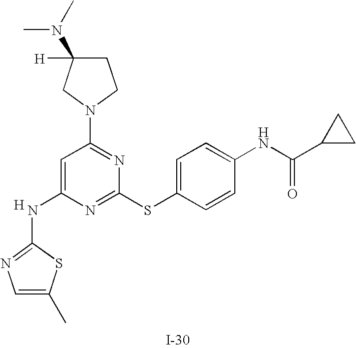

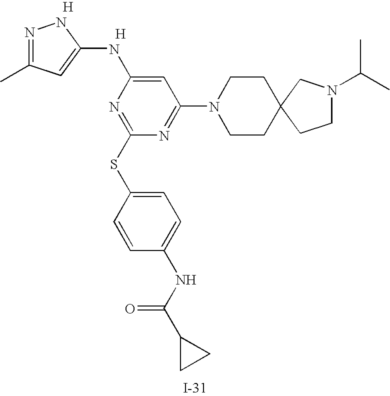

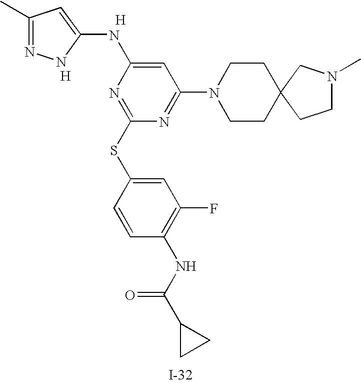

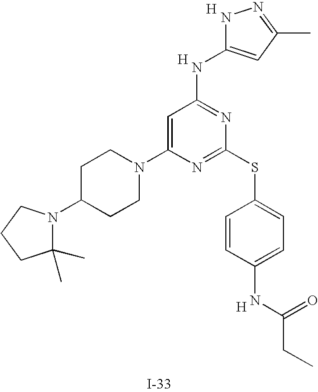

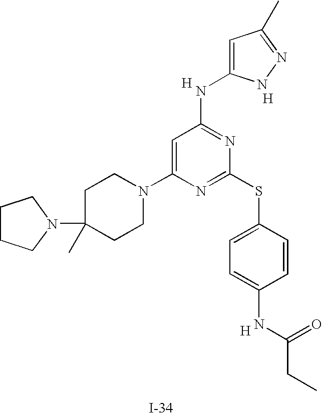

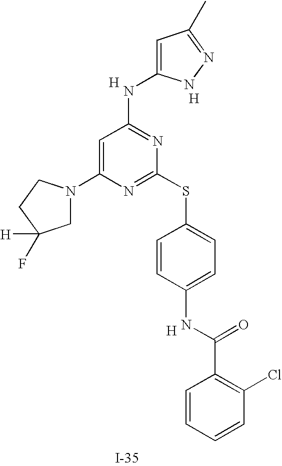

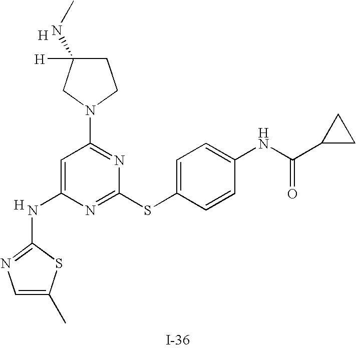

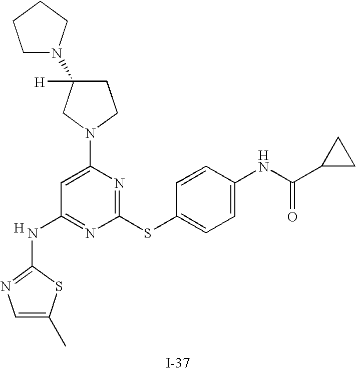

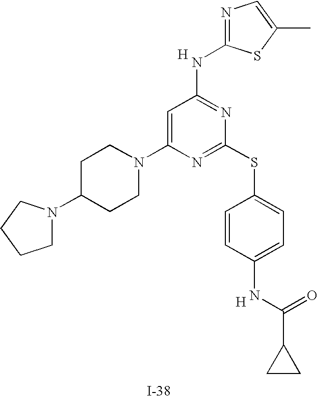

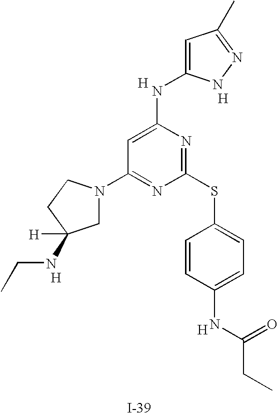

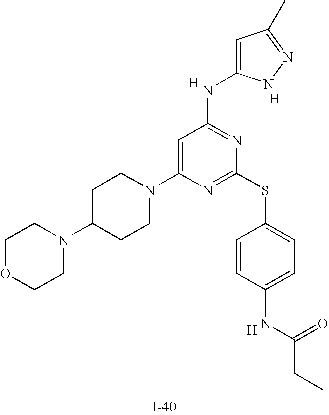

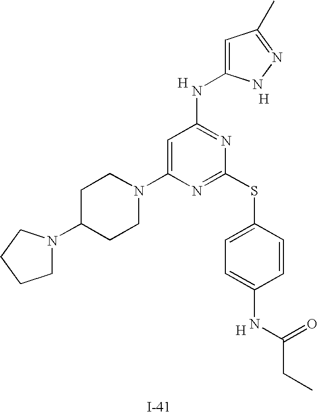

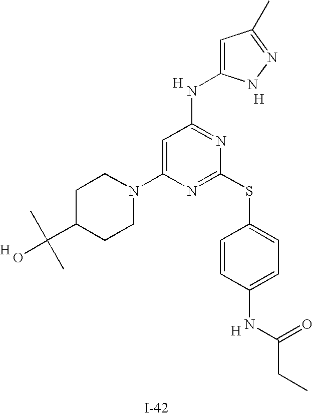

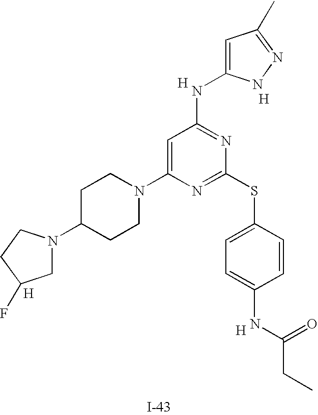

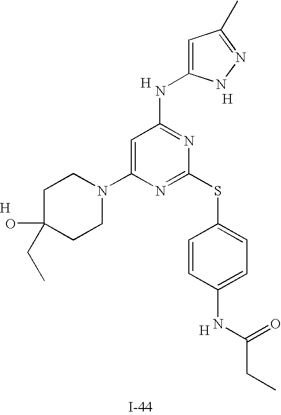

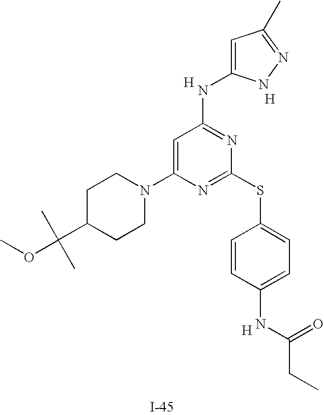

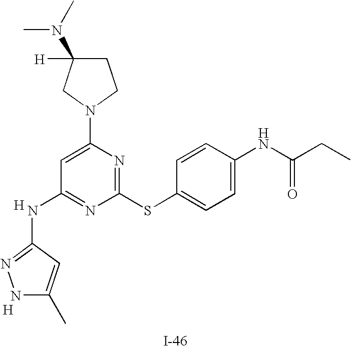

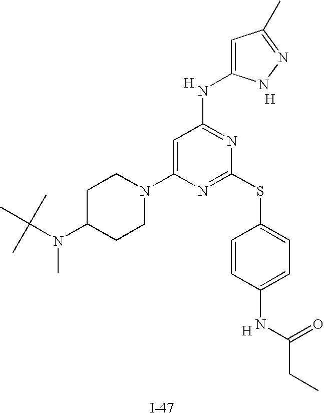

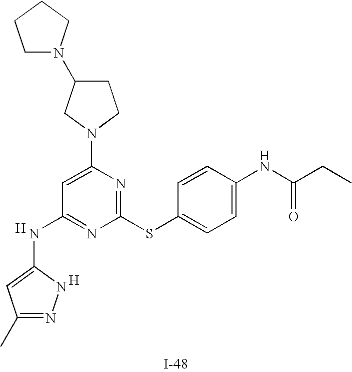

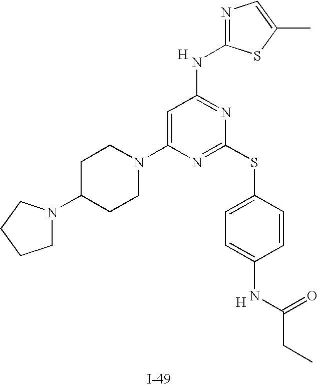

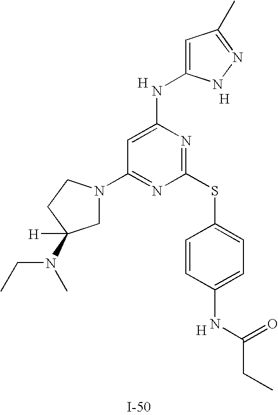

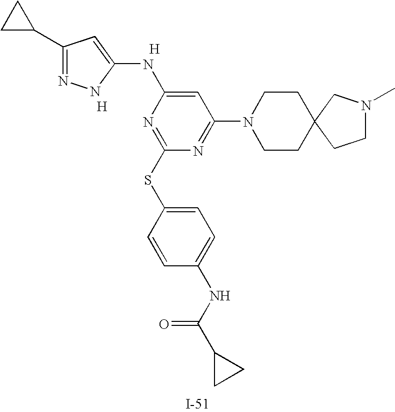

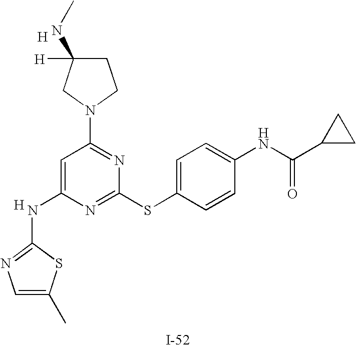

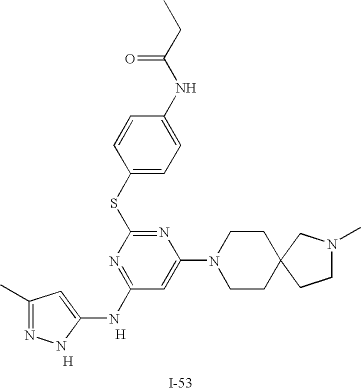

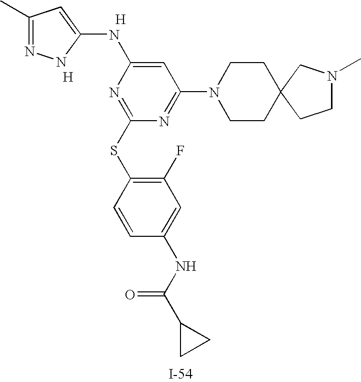

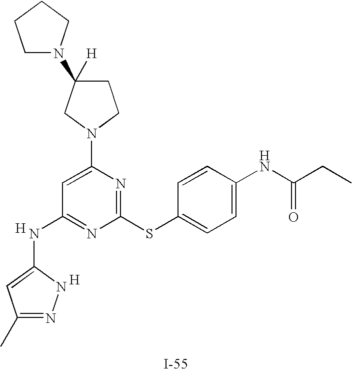

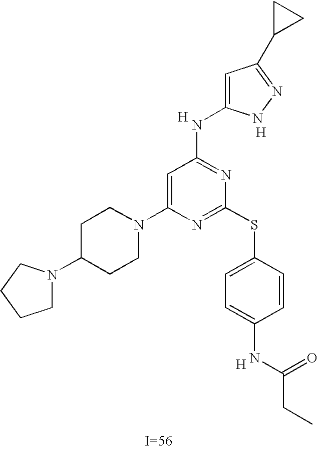

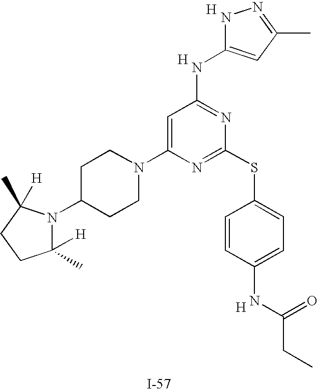

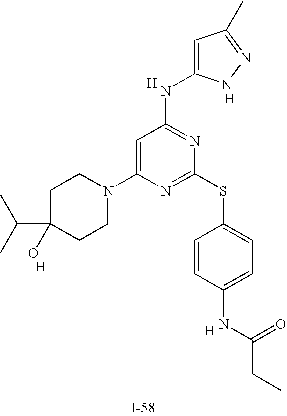

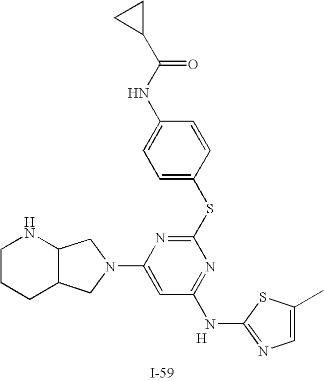

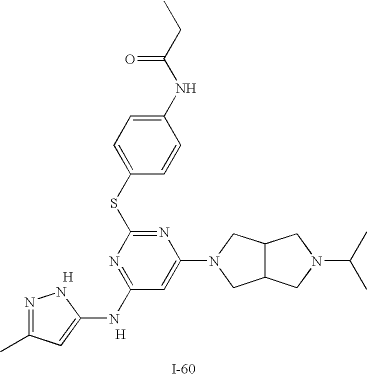

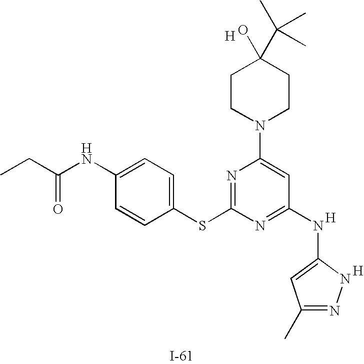

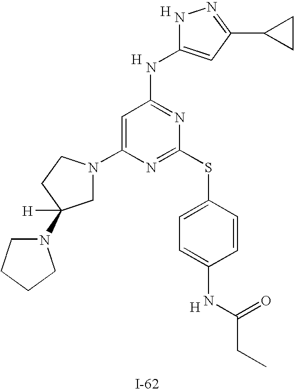

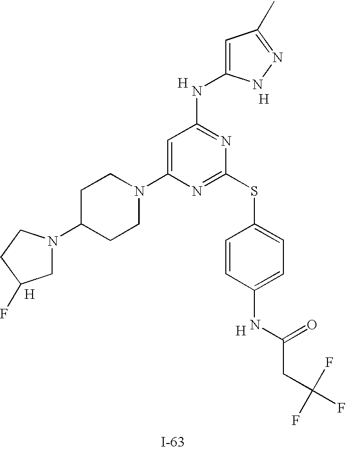

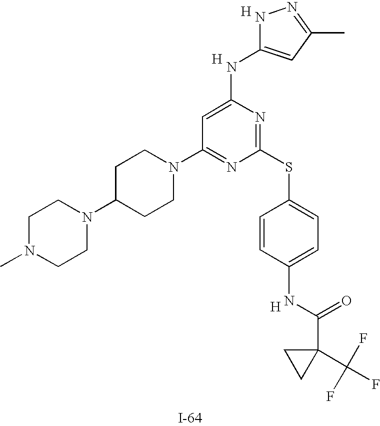

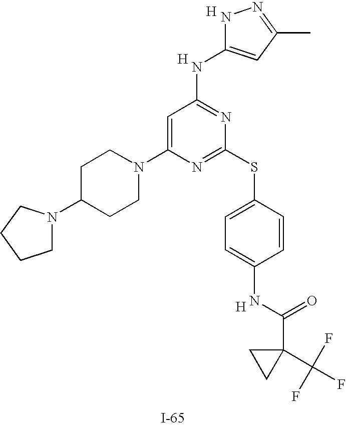

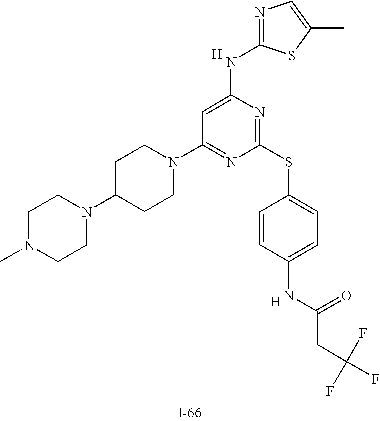

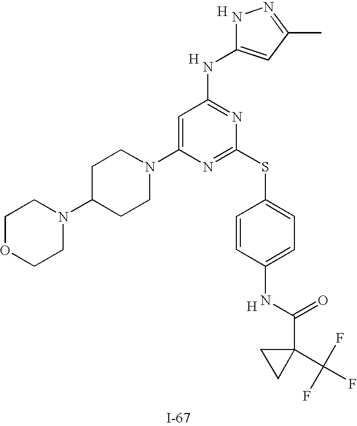

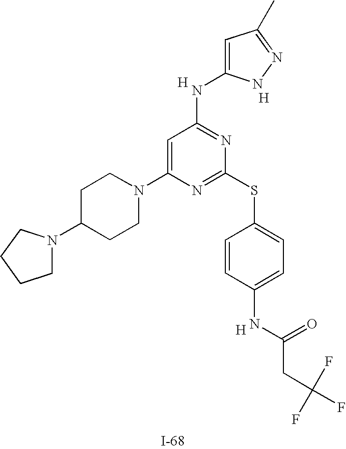

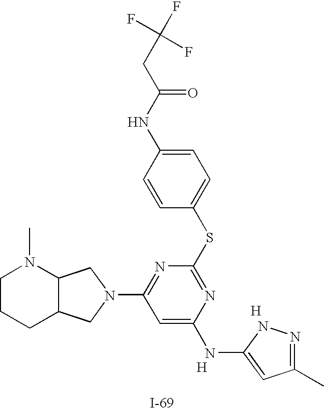

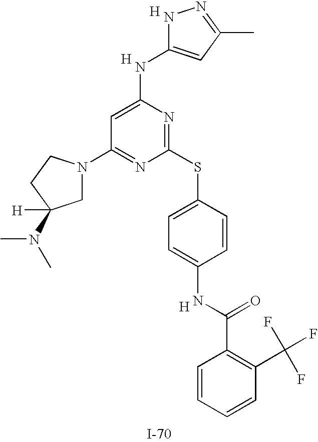

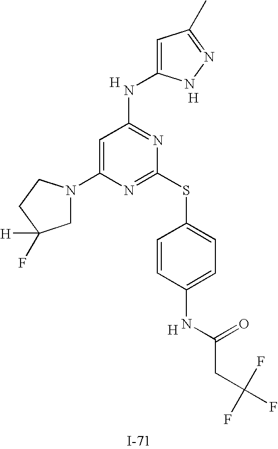

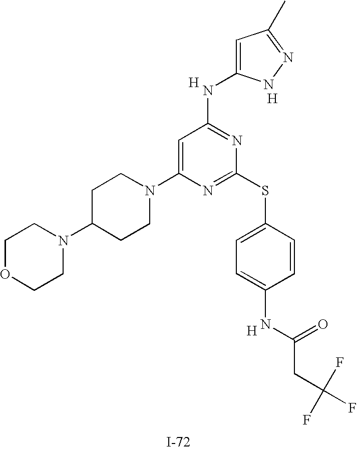

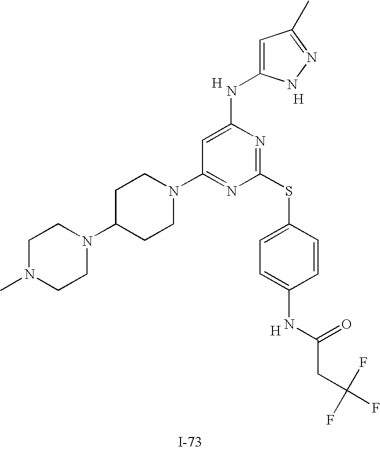

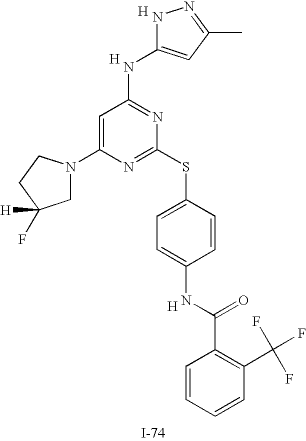

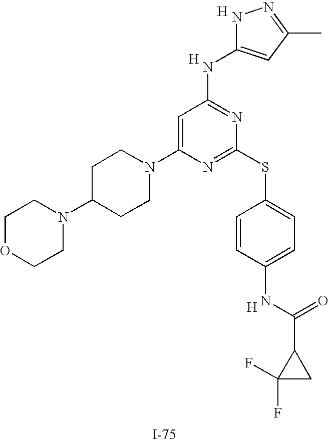

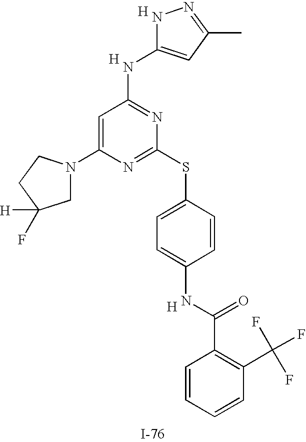

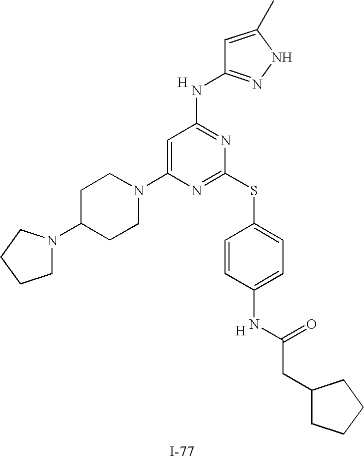

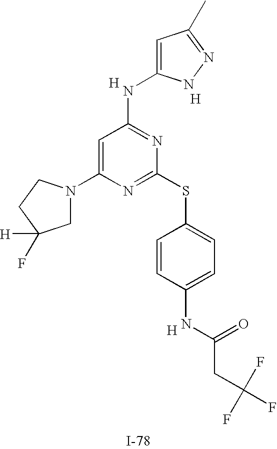

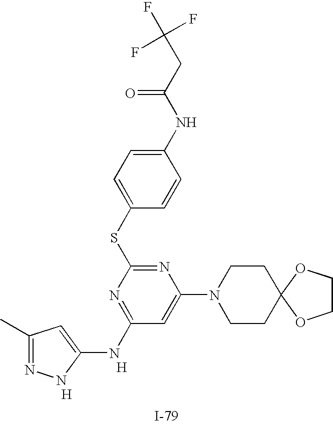

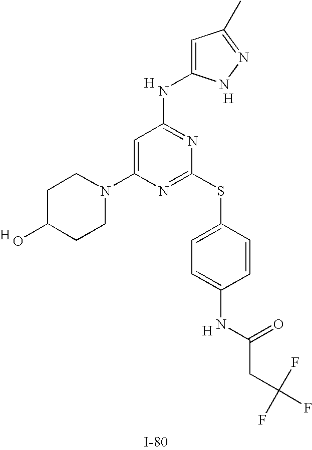

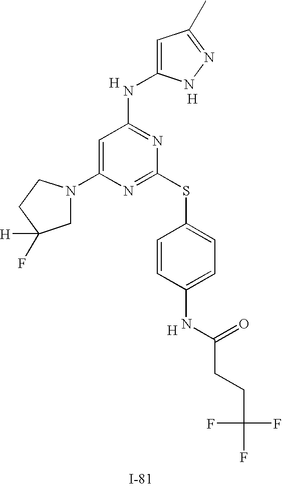

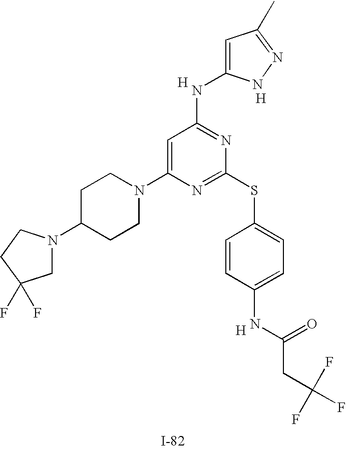

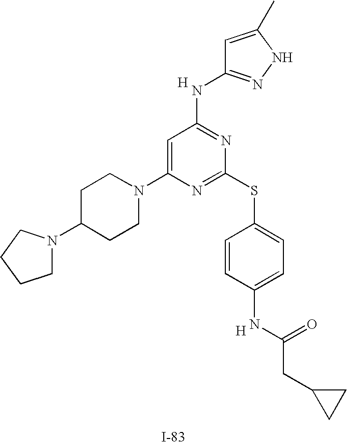

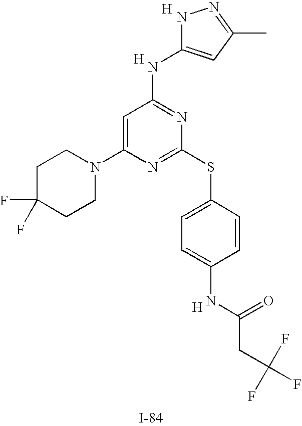

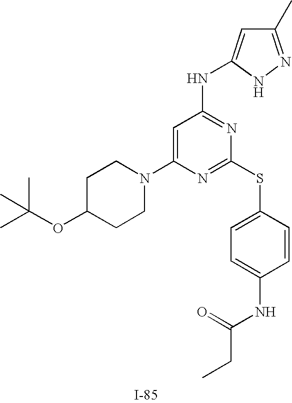

For purposes of this invention, the chemical elements are identified in accordance with the Periodic Table of the Elements, CAS version, Handbook of Chemistry and Physics, 75^{th} Ed. Additionally, general principles of organic chemistry are described in texts known to those of ordinary skill in the art, including, for example, “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As described herein, a specified number range of atoms includes any integer therein. For example, a group having from 1-4 atoms could have 1, 2, 3, or 4 atoms. A list of compounds expressed as “I-1 to I-5” means I-1, I-2, I-3, I-4, and I-5.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, and the like, as used herein, means an unbranched or branched, straight-chain or cyclic, substituted or unsubstituted hydrocarbon that is completely saturated or that contains one or more units of unsaturation that has a single point of attachment to the rest of the molecule. Suitable aliphatic groups include, but are not limited to, linear or branched, substituted or unsubstituted alkyl, alkenyl, or alkynyl groups. Specific examples include, but are not limited to, methyl, ethyl, isopropyl, n-propyl, sec-butyl, vinyl, n-butenyl, ethynyl, tert-butyl, cyclopropylmethyl, cyclopropylethyl, cyclobutylmethyl, cyclobutylethyl, cyclopentylmethyl, or cyclopentylethyl.

The term “cycloaliphatic” (or “carbocycle” or “carbocyclyl” or “cycloalkyl” and the like) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Suitable cycloaliphatic groups include, but are not limited to, cycloalkyl and cycloalkenyl groups. Specific examples include, but are not limited to, cyclohexyl, cyclopropenyl, and cyclobutyl.

The term “alkyl” as used herein, means an unbranched or branched, straight-chain or cyclic hydrocarbon that is completely saturated and has a single point of attachment to the rest of the molecule. Unless otherwise indicated, alkyl groups contain 1-12 carbon atoms. Specific examples of alkyl groups include, but are not limited to, methyl, ethyl, isopropyl, n-propyl, cyclopropyl, sec-butyl, and cyclobutyl.

In the compounds of this invention, rings include linearly-fused, bridged, or spirocyclic rings. Examples of bridged cycloaliphatic groups include, but are not limited to, bicyclo[3.3.2]decane, bicyclo[3.1.1]heptane, and bicyclo[3.2.2]nonane.

The term “heterocycle”, “heterocyclyl”, or “heterocyclic”, and the like, as used herein means non-aromatic, monocyclic or bicyclic ring in which one or more ring members are an independently selected heteroatom. In some embodiments, the “heterocycle”, “heterocyclyl”, or “heterocyclic” group has three to ten ring members in which one or more ring members is a heteroatom independently selected from oxygen, sulfur, nitrogen, or phosphorus, and each ring in the system contains 3 to 7 ring members. Examples of bridged heterocycles include, but are not limited to, 7-aza-bicyclo[2.2.1]heptane and 3-aza-bicyclo[3.2.2]nonane.

Suitable heterocycles include, but are not limited to, 3-1H-benzimidazol-2-one, 3-(1-alkyl)-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolane, benzodithiane, and 1,3-dihydro-imidazol-2-one.

As used herein, the term “Ht” is interchangeable with “Het” and

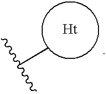

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “aryl” refers to monocyclic, or bicyclic ring having a total of five to twelve ring members, wherein at least one ring in the system is aromatic and wherein each ring in the system contains 3 to 7 ring members. The term “aryl” may be used interchangeably with the term “aryl ring”. The term “aryl” also refers to heteroaryl ring systems as defined hereinbelow.

The term “heteroaryl”, refers to monocyclic or bicyclic ring having a total of five to twelve ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”. Suitable heteroaryl rings include, but are not limited to, 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

The term “unsaturated”, as used herein, means that a moiety has one or more units of unsaturation.

The term “halogen” means F, Cl, Br, or I.

The term “protecting group”, as used herein, refers to an agent used to temporarily block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W., Wuts, P. G in “Protective Groups in Organic Synthesis”, Third Edition, John Wiley & Sons, New York: 1999, and other editions of this book, the entire contents of which are hereby incorporated by reference. The term “nitrogen protecting group”, as used herein, refers to an agents used to temporarily block one or more desired nitrogen reactive sites in a multifunctional compound. Preferred nitrogen protecting groups also possess the characteristics exemplified above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

Unless otherwise indicated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise indicated, all tautomeric forms of the compounds of the invention are within the scope of the invention. As would be understood by a skilled practitioner, a pyrazole group can be represented in a variety of ways. For example, a structure drawn as

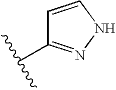

also represents other possible tautomers, such as

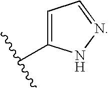

Likewise, a structure drawn as

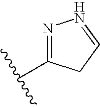

also represents other possible tautomers, such as

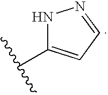

Unless otherwise indicated, a substituent can freely rotate around any rotatable bonds. For example, a substituent drawn as

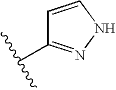

also represents

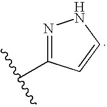

Likewise, a substituent drawn as

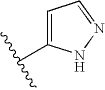

also represents

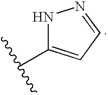

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

The compounds of this invention may be prepared in light of the specification using steps generally known to those of ordinary skill in the art. Those compounds may be analyzed by known methods, including but not limited to LCMS (liquid chromatography mass spectrometry) and NMR (nuclear magnetic resonance). It should be understood that the specific conditions shown below are only examples, and are not meant to limit the scope of the conditions that can be used for making compounds of this invention. Instead, this invention also includes conditions that would be apparent to those skilled in that art in light of this specification for making the compounds of this invention. Unless otherwise indicated, all variables in the following scheme are as defined herein

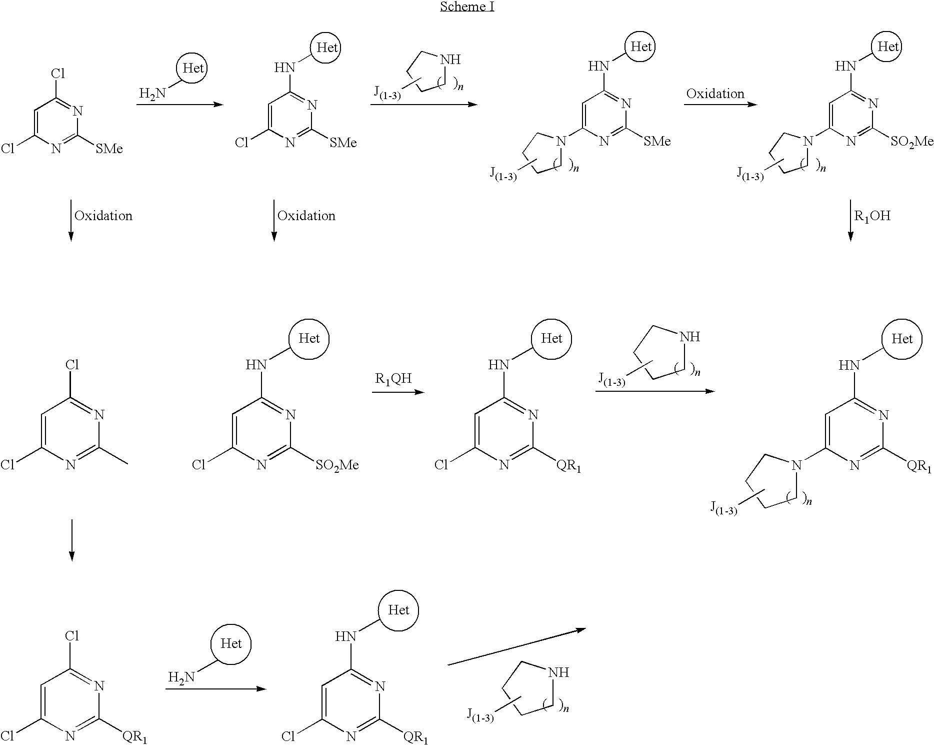

Scheme I above shows a generic method for making compounds of this invention. The compounds of this invention can be made in a variety of ways, as shown above. In essence, there are three main groups that are added to the dichloropyrimidine starting material. The order in which these groups are added can vary. The three main reactions involved are: addition of the pyrrolidine or piperidine, addition of the amino-heteroaryl, and addition of -Q-R¹ (which includes the oxidation of —SMe into a suitable leaving group, e.g., SO₂Me). As shown above, the pyrrolidine or piperidine, amino-heteroaryl, and -Q-R¹ can be added in various different orders. For instance, the amino-heteroaryl can be added first, followed by addition of the pyrrolidine or piperidine, oxidation, and finally addition of -Q-R¹. Or instead, oxidation can occur first, followed by addition of -Q-R¹, addition of the amino-heteroaryl, and finally addition of the pyrrolidine or piperidine. A skilled practitioner would understand the various reactions shown above.

The synthesis in the scheme above may be used to prepare compounds of this invention wherein R¹ is a ring substituted with 1 J¹ or 2-3 J² (the 1 J¹ or 2-3 J² being depicted above as 1-3 J groups).

Additionally, the compounds of this invention may be prepared according to the methods shown in WO 2004/000833.

Accordingly, this invention relates to processes for making the compounds of this invention.

Methods for evaluating the activity of the compounds of this invention (e.g., kinase assays) are known in the art and are also described in the examples set forth.

The activity of the compounds as protein kinase inhibitors may be assayed in vitro, in vivo or in a cell line. In vitro assays include assays that determine inhibition of either the kinase activity or ATPase activity of the activated kinase. Alternate in vitro assays quantitate the ability of the inhibitor to bind to the protein kinase and may be measured either by radiolabelling the inhibitor prior to binding, isolating the inhibitor/kinase complex and determining the amount of radiolabel bound, or by running a competition experiment where new inhibitors are incubated with the kinase bound to known radioligands.

Another aspect of the invention relates to inhibiting kinase activity in a biological sample, which method comprises contacting said biological sample with a compound of formula I or a composition comprising said compound. The term “biological sample”, as used herein, means an in vitro or an ex vivo sample, including, without limitation, cell cultures or extracts thereof; biopsied material obtained from a mammal or extracts thereof; and blood, saliva, urine, feces, semen, tears, or other body fluids or extracts thereof.

Inhibition of kinase activity in a biological sample is useful for a variety of purposes that are known to one of skill in the art. Examples of such purposes include, but are not limited to, blood transfusion, organ-transplantation, biological specimen storage, and biological assays.

Inhibition of kinase activity in a biological sample is also useful for the study of kinases in biological and pathological phenomena; the study of intracellular signal transduction pathways mediated by such kinases; and the comparative evaluation of new kinase inhibitors.

The Aurora protein kinase inhibitors or pharmaceutical salts thereof may be formulated into pharmaceutical compositions for administration to animals or humans. These pharmaceutical compositions, which comprise an amount of the Aurora protein inhibitor effective to treat or prevent an Aurora-mediated condition and a pharmaceutically acceptable carrier, are another embodiment of the present invention.

The term “Aurora-mediated condition” or “Aurora-mediated disease” as used herein means any disease or other deleterious condition in which Aurora (Aurora A, Aurora B, and Aurora C) is known to play a role. Such conditions include, without limitation, cancer, proliferative disorders, and myeloproliferative disorders.

Examples of myeloproliferative disorders include, but are not limited, to, polycythemia vera, thrombocythemia, myeloid metaplasia with myelofibrosis, chronic myelogenous leukaemia (CML), chronic myelomonocytic leukemia, hypereosinophilic syndrome, juvenile myelomonocytic leukemia, and systemic mast cell disease.

The term “cancer” also includes, but is not limited to, the following cancers: epidermoid Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal; rectum, Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma] hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; and Adrenal glands: neuroblastoma. Thus, the term “cancerous cell” as provided herein, includes a cell afflicted by any one of the above-identified conditions. In some embodiments, the cancer is selected from colorectal, thyroid, or breast cancer.

In some embodiments, the compounds of this invention are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease.

In some embodiments, the compounds of this invention are useful for treating hematopoietic disorders, in particular, acute-myelogenous leukemia (AML), chronic-myelogenous leukemia (CML), acute-promyelocytic leukemia (APL), and acute lymphocytic leukemia (ALL).

In addition to the compounds of this invention, pharmaceutically acceptable derivatives or prodrugs of the compounds of this invention may also be employed in compositions to treat or prevent the above-identified disorders.

A “pharmaceutically acceptable derivative or prodrug” means any pharmaceutically acceptable ester, salt of an ester or other derivative of a compound of this invention which, upon administration to a recipient, is capable of providing, either directly or indirectly, a compound of this invention or an inhibitorily active metabolite or residue thereof. Such derivatives or prodrugs include those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Examples of pharmaceutically acceptable prodrugs of the compounds of this invention include, without limitation, esters, amino acid esters, phosphate esters, metal salts and sulfonate esters.

The compounds of this invention can exist in free form for treatment, or where appropriate, as a pharmaceutically acceptable salt.

As used herein, the term “pharmaceutically acceptable salt” refers to salts of a compound which are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Base addition salts can be prepared by 1) reacting the purified compound in its acid form with a suitable organic or inorganic base and 2) isolating the salt thus formed.

Salts derived from appropriate bases include alkali metal (e.g., sodium and potassium), alkaline earth metal (e.g., magnesium), ammonium and N⁺ (C₁₋₄ alkyl)₄ salts. This invention also envisions the quaternization of any basic nitrogen-containing groups of the compounds disclosed herein. Water or oil-soluble or dispersible products may be obtained by such quaternization.

Base addition salts also include alkali or alkaline earth metal salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intraperitoneal, intrahepatic, intralesional and intracranial injection or infusion techniques.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, a bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, aqueous suspensions or solutions. In the case of tablets for oral use, carriers commonly used may include lactose and corn starch. Lubricating agents, such as magnesium stearate, may also be added. For oral administration in a capsule form, useful diluents may include lactose and dried cornstarch. When aqueous suspensions are required for oral use, the active ingredient may be combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials may include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations may be prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention may include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers may include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions may be prepared as solutions in saline, employing benzyl alcohol or other suitable preservatives, absorption promoters to enhance bioavailability, fluorocarbons, and/or other conventional solubilizing or dispersing agents.

The amount of kinase inhibitor that may be combined with the carrier materials to produce a single dosage form will vary depending upon the host treated, the particular mode of administration, and the indication. In an embodiment, the compositions should be formulated so that a dosage of between 0.01-100 mg/kg body weight/day of the inhibitor can be administered to a patient receiving these compositions. In another embodiment, the compositions should be formulated so that a dosage of between 0.1-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of inhibitor will also depend upon the particular compound in the composition.

According to another embodiment, the invention provides methods for treating or preventing cancer, a proliferative disorder, or a myeloproliferative disorder comprising the step of administering to a patient one of the herein-described compounds or pharmaceutical compositions.

The term “patient”, as used herein, means an animal, including a human.

In some embodiments, said method is used to treat or prevent a hematopoietic disorder, such as acute-myelogenous leukemia (AML), acute-promyelocytic leukemia (APL), chronic-myelogenous leukemia (CML), or acute lymphocytic leukemia (ALL).

In other embodiments, said method is used to treat or prevent myeloproliferative disorders, such as polycythemia vera, thrombocythemia, myeloid metaplasia with myelofibrosis, chronic myelogenous leukaemia (CML), chronic myelomonocytic leukemia, hypereosinophilic syndrome, juvenile myelomonocytic leukemia, and systemic mast cell disease.

In yet other embodiments, said method is used to treat or prevent cancer, such as cancers of the breast, colon, prostate, skin, pancreas, brain, genitourinary tract, lymphatic system, stomach, larynx and lung, including lung adenocarcinoma, small cell lung cancer, and non-small cell lung cancer.

Another embodiment provides a method of treating or preventing cancer comprising the step of administering to a patient a compound of formula I or a composition comprising said compound.

Another aspect of the invention relates to inhibiting kinase activity in a patient, which method comprises administering to the patient a compound of formula I or a composition comprising said compound. In some embodiments, said kinase is an Aurora kinase (Aurora A, Aurora B, Aurora C), Abl, Arg, FGFR1, MELK, MLK1, MuSK, Ret, or TrkA.

Depending upon the particular conditions to be treated or prevented, additional drugs may be administered together with the compounds of this invention. In some cases, these additional drugs are normally administered to treat or prevent the same condition. For example, chemotherapeutic agents or other anti-proliferative agents may be combined with the compounds of this invention to treat proliferative diseases.

Another aspect of this invention is directed towards a method of treating cancer in a subject in need thereof, comprising the sequential or co-administration of a compound of this invention or a pharmaceutically acceptable salt thereof, and a therapeutic agent. In some embodiments, said therapeutic agent is selected from an anti-cancer agent, an anti-proliferative agent, or a chemotherapeutic agent.

In some embodiments, said therapeutic agent is selected from camptothecin, the MEK inhibitor: U0126, a KSP (kinesin spindle protein) inhibitor, adriamycin, interferons, and platinum derivatives, such as Cisplatin.

In other embodiments, said therapeutic agent is selected from taxanes; inhibitors of bcr-abl (such as Gleevec, dasatinib, and nilotinib); inhibitors of EGFR (such as Tarceva and Iressa); DNA damaging agents (such as cisplatin, oxaliplatin, carboplatin, topoisomerase inhibitors, and anthracyclines); and antimetabolites (such as AraC and 5-FU).

In yet other embodiments, said therapeutic agent is selected from camptothecin, doxorubicin, idarubicin, Cisplatin, taxol, taxotere, vincristine, tarceva, the MEK inhibitor, U0126, a KSP inhibitor, vorinostat, Gleevec, dasatinib, and nilotinib.

In another embodiment, said therapeutic agent is dasatnib.

In another embodiment, said therapeutic agent is nilotinib.

In another embodiment, said therapeutic agent is selected from Her-2 inhibitors (such as Herceptin); HDAC inhibitors (such as vorinostat), VEGFR inhibitors (such as Avastin), c-KIT and FLT-3 inhibitors (such as sunitinib), BRAF inhibitors (such as Bayer's BAY 43-9006) MEK inhibitors (such as Pfizer's PD0325901); and spindle poisons (such as Epothilones and paclitaxel protein-bound particles (such as Abraxane®).

Other therapies or anticancer agents that may be used in combination with the inventive anticancer agents of the present invention include surgery, radiotherapy (in but a few examples, gamma-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), Gleevec™, dexamethasone, and cyclophosphamide.

A compound of the instant invention may also be useful for treating cancer in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); zoledronate (Zometa®) and vorinostat (Zolinza®).

For a comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

Another embodiment provides a simultaneous, separate or sequential use of a combined preparation.

Those additional agents may be administered separately, as part of a multiple dosage regimen, from the kinase inhibitor-containing compound or composition. Alternatively, those agents may be part of a single dosage form, mixed together with the kinase inhibitor in a single composition.

In order that this invention be more fully understood, the following preparative and testing examples are set forth. These examples are for the purpose of illustration only and are not to be construed as limiting the scope of the invention in any way. All documents cited herein are hereby incorporated by reference.

### EXAMPLES

As used herein, the term “Rt(min)” refers to the HPLC retention time, in minutes, associated with the compound. Unless otherwise indicated, the HPLC method utilized to obtain the reported retention time is as follows:

Column: ACE C8 column, 4.6×150 mm

Gradient: 0-100% acetonitrile+methanol 60:40 (20 mM Tris phosphate)

Flow rate: 1.5 mL/minute

Detection: 225 nm.

Mass spec. samples were analyzed on a MicroMass Quattro Micro mass spectrometer operated in single MS mode with electrospray ionization. Samples were introduced into the mass spectrometer using chromatography. Mobile phase for all mass spec. analyses consisted of 10 mM pH 7 ammonium acetate and a 1:1 acetonitrile-methanol mixture, column gradient conditions was 5%-100% acetonitrile-methanol over 3.5 mins gradient time and 5 mins run time on an ACE C8 3.0×75 mm column. Flow rate was 1.2 ml/min.

¹H-NMR spectra were recorded at 400 MHz using a Bruker DPX 400 instrument.

The following compounds of formula I were prepared according to the methods shown in the schemes described herein (Scheme I, Scheme II, Methods A, B, C, and D) and similar to the ones described herein for compound I-69. The compounds were also analyzed according to the methods described herein.

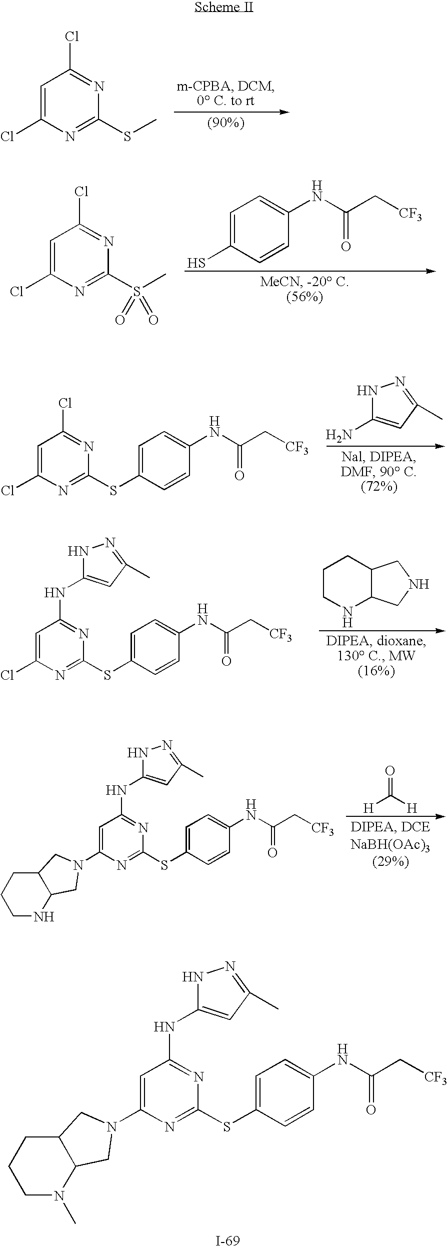

### Example 1

### 3,3,3-trifluoro-N-(4-(4-(3-methyl-1H-pyrazol-5-ylamino)-6-(1-methyltetrahydro-1H-pyrrolo[3,4-b]pyridin-6(2H,7H,7aH)-yl)pyrimidin-2-ylthio)phenyl)propanamide (I-69)

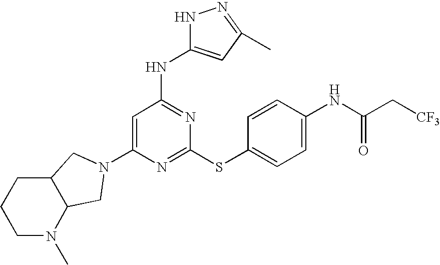

### Method A: 4,6-dichloro-2-(methylsulfonyl)pyrimidine

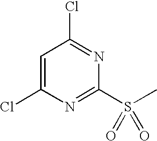

To a solution of 4,6-dichloro-2-(methylthio)pyrimidine (25 g, 0.13 mol) in dichloromethane (500 ml) at 0° C. was added m-chloroperbenzoic acid (74 g, 0.33 mol) over a period of 40 minutes. The solution was allowed to warm up to room temperature and stirred for a further 4 hours. The mixture was diluted with dichloromethane (750 ml) and then treated with 50% Na₂S₂O₃/NaHCO₃ solution, a saturated sodium bicarbonate solution and brine. The organic layer was dried over magnesium sulfate and concentrated in vacuo to afford the title compound as a white solid (26.75 g, 91% yield).

¹H NMR (DMSO D⁶, 400 MHz) δ 3.44 (3H, s), 8.43 (1H, s); MS (ES⁺) 229.

### Method B: N-(4-(4,6-dichloropyrimidin-2-ylthio)phenyl)-3,3,3-trifluoropropanamide

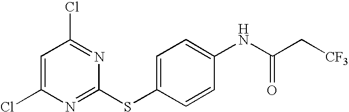

A solution of 4,6-dichloro-2-(methylsulfonyl)pyrimidine (8 g, 35 mmol) and 3,3,3-trifluoro-N-(4-mercaptophenyl)propanamide (8.7 g, 37 mmol) in acetonitrile (250 ml) was cooled down to −10° C. Triethylamine (4.9 ml, 35 mmol) was added dropwise over 20 minutes while maintaining the temperature at −10° C. Once added, the solution was stirred at that temperature for a further 20 minutes then allowed to warm up to room temperature and concentrated to 150 ml. Water (250 ml) was added to the reaction mixture. A solid was collected by filtration and dried by suction. This orange solid was slurried in a minimal amount of ethyl acetate. An off white solid was collected by filtration and dried in vacuo. The process was repeated to yield more solid. The batches were combined to give the desired compound (7.9 g, 56% yield). ¹H NMR (DMSO D⁶, 400 MHz) δ 3.59 (2H, q), 7.59 (2H, d), 7.70 (2H, d), 7.74 (1H, s), 10.58 (1H, s); MS (ES⁺) 383.

### Method C: N-(4-(4-chloro-6-(3-methyl-1H-pyrazol-5-ylamino)pyrimidin-2-ylthio)phenyl)-3,3,3-trifluoropropanamide

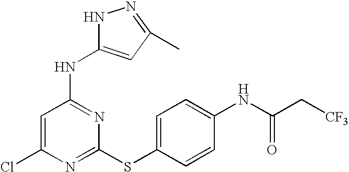

A solution of N-(4-(4,6-dichloropyrimidin-2-ylthio) phenyl)-3,3,3-trifluoro propanamide (14.2 g, 37 mmol), 3-amino-5-methylpyrazole (4 g, 41 mmol), sodium iodide (6.1 g, 41 mmol) and diisopropylethylamine (19.3 ml, 0.11 mol), in dimethylformamide (130 ml) was heated at 90° C. for 18 hours. The reaction mixture was concentrated to dryness. The residue was redissolved in ethyl acetate, washed with a saturated sodium bicarbonate aqueous solution and brine. The organic layer was dried over magnesium sulfate and concentrated in vacuo to afford an orange foam. The residue was slurried in dichloromethane and sonicated for 20 minutes. A solid was collected by filtration. This process was repeated to give more pure product. The pure batches were combined to give the desired product as a pale yellow solid (11.77 g, 72% yield). ¹H NMR (DMSO D⁶, 400 MHz) δ 1.96 (3H, s), 3.56 (2H, q), 5.26 (1H, br s), 6.49 (1H, br s), 7.59 (2H, d), 7.74 (2H, d), 10.21 (1H, br s), 10.57 (1H, br s), 11.90 (1H, br s); MS (ES⁺) 443.

### Method D: 3,3,3-trifluoro-N-(4-(4-(3-methyl-1H-pyrazol-5-ylamino)-6-(tetrahydro-1H-pyrrolo[3,4-b]pyridin-6(2H,7H,7aH)-yl)pyrimidin-2-ylthio)phenyl)propanamide

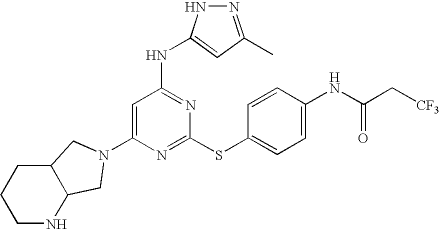

A microwave vial was charged with N-(4-(4-chloro-6-(3-methyl-1H-pyrazol-5-ylamino)pyrimidin-2-ylthio)phenyl)-3,3,3-trifluoropropanamide (2.5 g, 5.92 mmol), cis-Octahydropyrrolo[3,4-b]pyridine (2.23 g, 17.8 mmol), diisopropylethylamine (10.3 ml, 59.2 mmol) and dioxane (30 ml). The vial was heated at 130° C. for 90 minutes in the CEM microwave. The reaction mixture was diluted with ethyl acetate, washed with a saturated sodium bicarbonate solution and brine. The organic layer was dried over magnesium sulfate and concentrated in vacuo. The residue was purified by reverse phase preparative HPLC [Waters Sunfire C18, 10 μM, 100 A column, gradient 10%-95% B (solvent A: 0.05% TFA in water; solvent B: CH3CN) over 16 minutes at 25 mL/min] to give the desired product as a trifluoroacetic acid salt (620 mg, 16% yield). ¹H NMR (DMSO D⁶, 400 MHz) δ 1.60-1.82 (4H, m), 1.95-2.05 (4H, m), 2.92 (1H, m), 3.10-3.90 (7H, m, water peak obscures some signals), 5.43-5.85 (2H, m), 7.53 (2H, d), 7.69 (2H, d), 8.32 (1H, br s), 8.83 (1H, br d), 9.24 (1H, s), 10.5 (1H, s), 11.69 (1H, br s).

### Method E: 3,3,3-trifluoro-N-(4-(4-(3-methyl-1H-pyrazol-5-ylamino)-6-(1-methyltetrahydro-1H-pyrrolo[3,4-b]pyridin-6(2H,7H,7aH)-yl)pyrimidin-2-ylthio)phenyl)propanamide

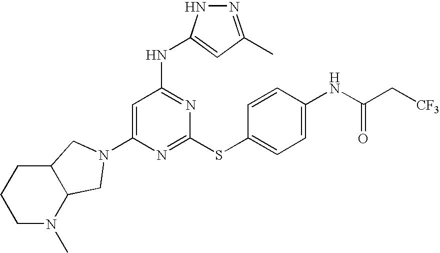

Sodium triacetoxyborohydride (382 mg, 1.8 mmol) was added to a suspension of 3,3,3-trifluoro-N-(4-(4-(3-methyl-1H-pyrazol-5-ylamino)-6-(tetrahydro-1H-pyrrolo[3,4-b]pyridin-6(2H,7H,7aH)-yl)pyrimidin-2-ylthio)phenyl)propanamide 2,2,2-trifluoroacetate (584 mg, 0.90 mmol), diisopropylethylamine (0.313 ml, 1.80 mmol) and 37% formaldehyde (0.073 ml, 0.90 mmol) in dichloroethane (30 ml). The reaction mixture was stirred at room temperature for 20 minutes. The reaction was quenched by addition of a saturated solution of sodium bicarbonate. The aqueous phase was extracted with dichloromethane (3 times). The combined organic layers were dried over sodium sulfate and concentrated in vacuo. The residue was purified on silica gel by flash column chromatography and recrystallised from hot EtOAc/cyclohexane to give the desired product as a white solid (144.1 mg, 29% yield). ¹H-1 NMR (DMSO D⁶, 400 MHz) δ 1.40-1.51 (1H, m), 1.52-1.69 (3H, m), 2.01 (3H, brs), 2.01-2.09 (1H, partly obscured m), 2.15 (3H, brs), 2.30-2.41 (1H, m), 2.06-2.68 (2H, m), 3.11-3.28 (2H, m), 3.53 (2H, q), 5.45 (1H, brs), 5.77 (1H, vbrs), 0.54 (2H, d), 7.67 (2H, d), 9.07 (1H, brs), 10.48 (1H, s), 11.65 (1H, brs). NB solvent/water peaks obscure some signals.

The various R^{y}H moieties used in the preparation of compounds of formula I are either commercially available (4-(Piperidin-4-yl)-morpholine; (S)-(−)-3-pyrrolidinol; (R)-(+)-3-Pyrrolidinol; (S)-(−)-3-(Methylamino)pyrrolidine; (R)-(+)-3-(Methylamino)pyrrolidine; 1,4-Dioxa-8-azaspiro[4.5]decane; 4-Cyanopiperidine; 4-(trifluoromethyl)piperidine; 4-Piperidone monohydrate hydrochloride; (R)-(−)-3-Fluoropyrrolidine hydrochloride; (S)-(+)-3-Fluoropyrrolidine hydrochloride; 4-methylpiperidin-4-ol hydrochloride; 4-tert-Butyl-piperidine; (3S)-(−)-3-(Dimethylamino)pyrrolidine; (3R)-(+)-3-(Dimethylamino)pyrrolidine; 3,3-Difluoropiperidine hydrochloride; 3,3-Difluoropyrrolidine hydrochloride; 4-(1-Pyrrolidinyl)piperidine; (3S)-(−)-3-(Ethylamino)pyrrolidine; [1,3′]Bipyrrolidinyl; 1-Methyl-4-(piperidin-4-yl)piperazine; 4-Hydroxypiperidine; 4,4-Difluoropiperidine), described in the literature (See Palmer, J. T.; et al. J. Med. Chem., 2005, 48, 7520 for the synthesis of tert-butyl-piperidin-4-yl-amine; Osakada, K.; Ikariya, T.; Saburi, M.; Yoshikawa, S.; Chem. Lett., 1981, 1691 for the synthesis of (S)—N-methylpiperidin-3-amine; U.S. Pat. No. 5,521,199 for the synthesis of 2-(piperidin-4-yl)propan-2-ol) or can be prepared following procedures similar to the ones described below.

### Example 2

### (R)—N-isopropylpyrrolidin-3-amine bis(2,2,2-trifluoroacetate)

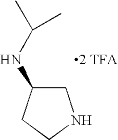

### Method F: (R)-tert-butyl 3-(propan-2-ylideneamino)pyrrolidine-1-carboxylate

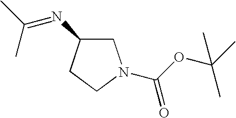

(3R)-3-Amino-1-(tert-butoxycarbonyl)pyrrolidine (0.5 g, 2.69 mmol) was dissolved in a mixture of dichloromethane (10 ml) and acetone (2 ml). Magnesium sulfate (0.5 g) was added and the reaction mixture was stirred at room temperature for 18 hours. The mixture was filtered and concentrated under reduced pressure to afford the title compound as an oil (601 mg, 99% yield). ¹H NMR (CDCl₃, 400 MHz) δ 1.46 (9H, s), 2.10 (4H, m), 3.10 (1H, dd), 3.33-3.65 (4H, m).

### Method G: (R)-tert-butyl 3-(isopropylamino)pyrrolidine-1-carboxylate

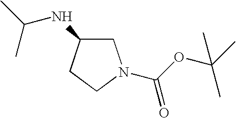

Platinum oxide (60 mg) was added to a solution of (R)-tert-butyl 3-(propan-2-ylideneamino)pyrrolidine-1-carboxylate (600 mg, 2.65 mmol) in methanol (4 ml). The reaction was stirred for 18 hours under an atmosphere of hydrogen. The reaction mixture was filtered through a path of celite and washed with more methanol. The filtrate was concentrated in vacuo to afford an oil which solidified on standing (320 mg, 53% yield). ¹H NMR (CDCl₃, 400 MHz) δ 1.02 (6H, d), 1.38 (9H, s), 1.58-1.72 (2H, m), 2.02 (1H, m), 2.82 (1H, m), 3.08 (1H, m), 3.20 (1H, m), 3.35 (1H, m), 3.40-3.60 (2H, m).

### Method H: (R)—N-isopropylpyrrolidin-3-amine bis(2,2,2-trifluoroacetate)

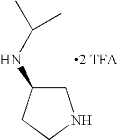

Trifluoroacetic acid (2 ml) was added to (R)-tert-butyl 3-(isopropylamino)pyrrolidine-1-carboxylate (520 mg, 2.28 mmol) in dichloromethane (3 ml). The reaction was stirred for 7 hours at room temperature. The reaction mixture was concentrated in vacuo. The residue was triturated with petroleum ether to afford the desired compound as a solid (721 mg, 89% yield). ¹H NMR (DMSO D⁶, 400 MHz) δ 1.25 (6H, d), 2.02 (1H, m), 2.31 (1H, m), 3.12-3.70 (5H, m), 3.85-4.10 (1H, m), 8.90-9.50 (4H, br m).

Other R^{y}H moieties used in the preparation of compounds of formula I can be prepared via a sequence similar to the one described example 2 (methods F, G and H): (S)—N-isopropylpyrrolidin-3-amine bis(2,2,2-trifluoroacetate)

### Example 3

### 3-neopentylpyrrolidin-3-ol 2,2,2-trifluoroacetate

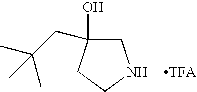

### Method I: tert-butyl 3-hydroxy-3-neopentylpyrrolidine-1-carboxylate

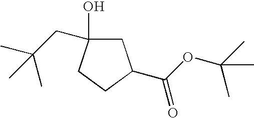

Cerium(III) chloride heptahydrate (3.42 g, 9.17 mmol) was heated under high vacuum at 140° C. for 18 hours. Argon was introduced into the hot flask and then cooled to 0° C. before tetrahydrofuran (30 ml) was added with rapid stirring. The suspension was then allowed to warm to room temperature and stirred for 18 hours. The suspension was cooled to 0° C. and neopentylmagnesium chloride 1M in diethylether (9.17 ml, 9.17 mmol) was added and stirred at 0° C. for 90 minutes. N-(tert-butoxycarbonyl)-3-pyrrolidinone (1.13 g, 6.11 mmol) in tetrahydrofuran (10 ml) was added dropwise at 0° C. The reaction mixture was stirred at 0° C. for a further 2 hours after complete addition. The reaction mixture was quenched with a saturated solution of ammonium chloride, extracted with ethyl acetate. The organic layer was washed with brine, dried over magnesium sulfate and concentrated in vacuo. The crude product was purified by silica gel flash chromatography to afford the title compound as a white solid (281 mg, 19% yield). ¹H NMR (CDCl₃, 400 MHz) δ 1.07 (9H, s), 1.48 (9H, s), 1.55 (1H, br s), 1.65 (2H, dd), 1.85 (1H, m), 1.96 (1H, tq), 3.26 (1H, d), 3.41-3.53 (3H, m).

### Method J: 3-neopentylpyrrolidin-3-ol 2,2,2-trifluoroacetate

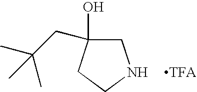

Trifluoroacetic acid (1 ml) was added to a solution of tert-butyl 3-hydroxy-3-neopentylpyrrolidine-1-carboxylate (281 mg, 1.14 mmol) in dichloromethane (8 ml) at 0° C. The reaction was stirred for 2 hours at 0° C. The reaction mixture was concentrated in vacuo to afford the desired compound as a brown oil (281 mg, 91% yield). ¹H NMR (CDCl₃, 400 MHz) δ 1.07 (9H, s), 1.74 (2H, dd), 2.03 (1H, m), 2.25 (1H, m), 3.07 (1H, m), 3.46-3.63 (3H, m), 8.66 (1H, br s), 9.10 (1H, br s).

Other R^{y}H moieties used in the preparation of compounds of formula I can be prepared via a sequence similar to the one described example 3 (methods I and J): 3-tert-butylpyrrolidin-3-ol 2,2,2-trifluoroacetate; 4-ethylpiperidin-4-ol 2,2,2-trifluoroacetate; 4-isopropylpiperidin-4-ol 2,2,2-trifluoroacetate; 4-tert-butylpiperidin-4-ol 2,2,2-trifluoroacetate.

### Example 4

### 4-((2R,5R)-2,5-dimethylpyrrolidin-1-yl)piperidine 2,2,2-trifluoroacetate

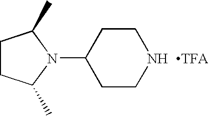

### Method K: tert-butyl 4-((2R,5R)-2,5-dimethylpyrrolidin-1-yl)piperidine-1-carboxylate

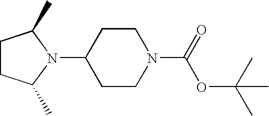

Sodium cyanoborohydride (315 mg, 5.01 mmol) was added in one portion to a stirred solution of 1-tert-Butoxycarbonyl-piperidin-4-one (1 g, 5.01 mmol) and (2R,5R)-(−) -trans-2,5-dimethylpyrrolidine (0.5 g, 5.01 mmol) in trifluoroethanol (12 ml). The reaction mixture was stirred at room temperature for 3 hours. It was hydrolysed with a saturated sodium bicarbonate aqueous solution. Extractions were carried out with ethyl acetate. The organic layer was washed with water and brine, dried over magnesium sulfate and concentrated in vacuo. The residue was purified on silica gel by column flash chromatography (20% ethyl acetate in petroleum) to afford the title compound as an oil (400 mg, 28% yield). ¹H NMR (CDCl₃, 400 MHz) δ 1.02 and 1.07 (6H, d, rotamers), 1.32-1.45 (2H, m), 1.47 and 1.48 (9H, s, rotamers), 1.51-1.65 (1H, m), 1.67-1.95 (3H, m), 2.01-2.12 (2H, m), 2.66-2.76 (3H, m), 3.25-3.35 (2H, m), 4.05-4.25 (2H, m).

### 4-((2R,5R)-2,5-dimethylpyrrolidin-1-yl)piperidine 2,2,2-trifluoroacetate

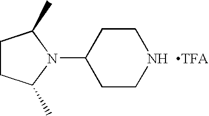

4-((2R,5R)-2,5-dimethylpyrrolidin-1-yl)piperidine 2,2,2-trifluoroacetate was prepared by using method J.

Other R^{y}H moieties used in the preparation of compounds of formula I can be prepared via a sequence similar to the one described example 4 (methods K and J): 2,2-dimethyl-1,3′-bipyrrolidine 2,2,2-trifluoroacetate; 4-(2,2-dimethylpyrrolidin-1-yl)piperidine 2,2,2-trifluoroacetate; (R)-4-(3-fluoropyrrolidin-1-yl)piperidine 2,2,2-trifluoroacetate; (S)-4-(3-fluoropyrrolidin-1-yl)piperidine 2,2,2-trifluoroacetate; 4-(3,3-difluoropyrrolidin-1-yl)piperidine 2,2,2-trifluoroacetate.

### Example 5

### (S)-3-isopropoxypyrrolidine 2,2,2-trifluoroacetate

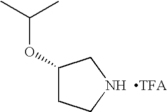

### Method L: (S)-tert-butyl 3-isopropoxypyrrolidine-1-carboxylate

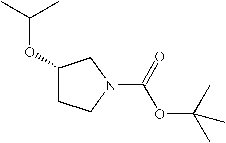

To a stirred solution of (S)-(+)-tert-butyl-3-hydroxypyrrolidine-1-carboxylate (1.0 g, 5.34 mmol) in isopropyl iodide (15 ml) was added silver(I) oxide (1.49 g, 6.41 mmol). The reaction mixture stirred at room temperature for 24 hours then heated at 40° C. for a further 24 hours. The reaction mixture was then cooled down to room temperature and filtered through a pad of Celite, washing with diethyl ether (2×10 ml). The filtrate was concentrated in vacuo and the residue purified on silica gel by flash column chromatography eluting with 20% ethyl acetate in petroleum ether to afford the title compound as a colourless oil (0.50 g, 41% yield). ¹H NMR (CDCl₃, 400 MHz) δ 1.02 and 1.07 (6H, d, rotamers), 1.32-1.45 (2H, m), 1.47 and 1.48 (9H, s, rotamers), 1.51-1.65 (1H, m), 1.67-1.95 (3H, m), 2.01-2.12 (2H, m), 2.66-2.76 (3H, m), 3.25-3.35 (2H, m), 4.05-4.25 (2H, m).

### (S)-3-isopropoxypyrrolidine 2,2,2-trifluoroacetate

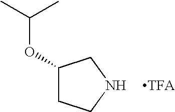

(S)-3-isopropoxypyrrolidine 2,2,2-trifluoroacetate was prepared from (S)-tert-butyl 3-isopropoxypyrrolidine-1-carboxylate by using method J.

### Example 6

### (R)—N,N-dimethylpiperidin-3-amine hydrochloride

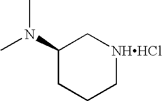

### Method M: (S)-tert-butyl 3-(methylsulfonyloxy)piperidine-1-carboxylate

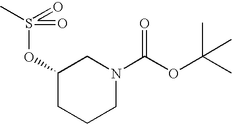

To a stirred solution of (S)-(+)-tert-butyl-3-hydroxypyrrolidine-1-carboxylate (10.0 g, 49.7 mmol) and triethylamine (13.9 ml, 99.4 mmol) in dichloromethane (150 ml) was added methanesulfonyl chloride (4.25 ml, 54.7 mmol) at 0° C. The reaction mixture was allowed to warm up to room temperature and stirred for 18 hours. The reaction mixture was washed with a saturated solution of sodium bicarbonate, water and brine. The organic layer was dried over magnesium sulfate and concentrated in vacuo to provide the title compound as a white solid (11.78 g, 85% yield). ¹H NMR (CDCl₃, 400 MHz) δ 1.48 (9H, s), 1.55 (1H, br s), 1.76-2.05 (3H, m), 3.07 (3H, s), 3.35 (1H, m), 3.48 (1H, m), 3.53-3.74 (2H, m), 4.73 (1H, br s).

### Method N: (R)-tert-butyl 3-(dimethylamino)piperidine-1-carboxylate

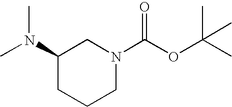

A solution of (S)-tert-butyl 3-(methylsulfonyloxy)piperidine-1-carboxylate (1.0 g, 3.58 mmol) and 2 M dimethylamine in methanol (60 ml) was placed in a sealed tube and heated to 90° C. for 48 hours. The reaction mixture was allowed to cool down to room temperature and was concentrated in vacuo. Ethyl acetate was added to the residue. The remaining mesylate crashed out and was removed by filtration. The filtrate was concentrated in vacuo to give the desired compound as a sticky orange oil (0.808 g, 99% yield).

### Method O: (R)—N,N-dimethylpiperidin-3-amine hydrochloride

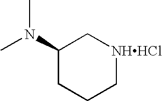

1.25 M hydrochloric acid in methanol (13 ml) was added to a solution of (R)-tert-butyl 3-(dimethylamino) piperidine-1-carboxylate (808 mg, 3.54 mmol) in methanol (5 ml). The reaction mixture was stirred at room temperature for 3 hours, then, concentrated in vacuo to leave the desired product as an orange oil (0.737 g, quantitative yield).

Other R^{y}H moieties used in the preparation of compounds of formula I can be prepared via a sequence similar to the one described in example 6 (methods M, N and O): (S)—N,N-dimethylpiperidin-3-amine hydrochloride; (R)-1,3′-bipyrrolidine hydrochloride, (S)—N-ethyl-N-methylpyrrolidin-3-amine hydrochloride, (S)-1,3′-bipyrrolidine hydrochloride.

Other R^{y}H moieties comprised in compounds of formula I of this invention can be prepared via method O: octahydro-1H-pyrrolo[3,4-b]pyridine dihydrochloride.

### Example 7

### 3-methylpyrrolidin-3-ol

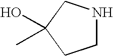

### Method P: 1-benzyl-3-methylpyrrolidin-3-ol

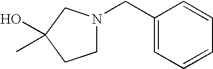

A solution of 1-benzyl-3-pyrrolidinone (2 ml, 12.2 mmol) in diethylether (20 ml) was added dropwise to a stirred solution of 3 M methyl magnesium bromide (5.29 ml, 15.86 mmol) in diethylether (10 ml) and tetrahydrofuran (5 ml) at 0° C. The reaction mixture was stirred at 0° C. for a further 40 minutes. The reaction mixture was quenched with an aqueous solution of ammonium chloride and the compound was extracted into ethyl acetate. The organic phase was washed with brine, dried over magnesium sulfate and concentrated in vacuo. The residue was purified on silica gel by flash column chromatography to afford the title compound as a yellow oil (1.04 g, 45% yield). ¹H NMR (CDCl₃, 400 MHz) δ 1.37 (3H, s), 1.87-2.00 (2H, m), 2.32 (1H, d), 2.43 (1H, q), 2.66 (1H, br s), 2.80 (1H, d), 3.05 (1H, dt), 3.71 (2H, s), 7.26-7.33 (5H, m).

### Method Q: 3-methylpyrrolidin-3-ol

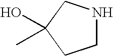

A suspension of 1-benzyl-3-methylpyrrolidin-3-ol (1.04 g, 5.44 mmol) and 10% palladium on carbon (50% wet) (300 mg) in methanol was shaken under hydrogen atmosphere in a Parr bottle at 60 psi for 48 hours. The reaction mixture was filtered through a short path of celite washing with methanol. The filtrate was concentrated in vacuo to give an orange oil (472 mg, 86% yield). ¹H NMR (CDCl₃, 400 MHz) δ 1.38 (3H, s), 1.77-1.97 (2H, m), 2.21 (2H, br s), 2.76 (1H, d), 2.98-3.10 (2H, m), 3.22-3.30 (1H, m).

### Example 8

### (S)-3-methoxypyrrolidine hydrochloride

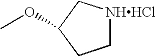

### Method R: (S)-tert-butyl 3-methoxypyrrolidine-1-carboxylate

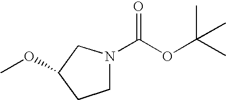

Sodium hydride (60%, 256 mg, 6.40 mmol) was added portionwise to a solution of (S)—N-(tert-Butoxycarbonyl)-3-hydroxypyrrolidine (1 g, 5.34 mmol) in tetrahydrofuran (30 ml) at 0° C. The reaction mixture was warmed up to room temperature and stirred for 30 minutes. The reaction was cooled down to 0° C. and methyl iodide (0.7 ml, 10.68 mmol) was added. The mixture was warmed up to room temperature and stirred for a further 18 hours. Water (20 ml) and diethylether (20 ml) were added. The aqueous layer was further extracted with diethylether. The combined organic layers were dried over magnesium sulfate and concentrated in vacuo to afford the title compound as a pale yellow oil (0.794 mg, 74% yield).

### (S)-3-methoxypyrrolidine hydrochloride

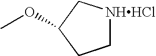

(S)-3-methoxypyrrolidine hydrochloride was prepared from (S)-tert-butyl 3-methoxypyrrolidine-1-carboxylate by using method O.

### Example 9

### 2-isopropyl-2,8-diazaspiro[4.5]decane bis(2,2,2-trifluoroacetate)

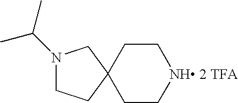

### Method S: tert-butyl 2-isopropyl-2,8-diazaspiro[4.5]decane-8-carboxylate

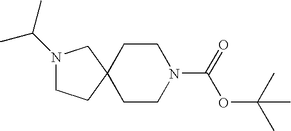

A solution of tert-butyl 2,8-diazaspiro[4.5]decane-8-carboxylate (1.5 g, 6.25 mmol) (prepared following the procedure described in the literature: US20060019985), sodium triacetoxy borohydride (2.1 g, 10 mmol) and acetic acid (2 drops) in acetone (20 ml) was stirred at room temperature for 16 hours. The reaction mixture was hydrolysed with a saturated aqueous solution of sodium bicarbonate (20 ml) and the compound was extracted with ethyl acetate (3×50 ml). The organic layer was washed with brine, dried over magnesium sulfate and concentrated under reduced pressure. The residue was purified on silica gel by flash column chromatography to afford the desired compound (150 mg, 8% yield). ¹H NMR (CD₃OD, 400 MHz) δ 1.33-1.36 (6H, d), 1.45 (9H, s), 1.60-1.65 (4H, m), 1.95-2.05 (2H, m), 3.30-3.40 (2H, m), 3.45-3.55 (2H, m).

### 2-isopropyl-2,8-diazaspiro[4.5]decane bis(2,2,2-trifluoroacetate)

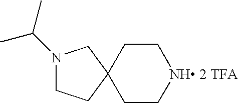

2-isopropyl-2,8-diazaspiro[4.5]decane bis(2,2,2-trifluoroacetate) was prepared from tert-butyl 2-isopropyl-2,8-diazaspiro[4.5]decane-8-carboxylate by using method J.

Other R^{y}H moieties used in the preparation of compounds of formula I can be prepared via a sequence similar to the one described in example 9 (methods S and J): 2-isopropyl-2,7-diazaspiro[4.4]nonane bis(2,2,2-trifluoroacetate), 2-isopropyloctahydropyrrolo[3,4-c]pyrrole bis(2,2,2-trifluoroacetate).

### Example 10

### 2-methyl-2,8-diazaspiro[4.5]decane hydrochloride

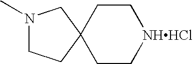

### Method T: tert-butyl 2-methyl-1-oxo-2,8-diazaspiro[4.5]decane-8-carboxylate

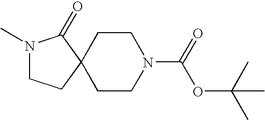

A solution of 4-spiro-[3-(N-methyl-2-pyrrolidinone)]-piperidine hydrochloride (1.0 g, 5 mmol), di-tert-butyl dicarbonate (1.4 g, 6 mmol) and triethylamine (1.7 ml, 12 mmol) in dichloromethane (20 ml) was stirred at room temperature for 18 hours. The reaction mixture was diluted with dichloromethane, washed with a saturated aqueous solution of sodium bicarbonate and brine. The organic layer was dried over magnesium sulfate and concentrated under reduced pressure. The residue was purified on silica gel by flash column chromatography to afford the desired compound (1.3 g, 99% yield).

¹H NMR (DMSO D⁶, 400 MHz) δ 1.26-1.35 (2H, m), 1.40 (9H, s), 1.52 (2H, dt), 1.91 (2H, t), 2.72 (3H, s), 2.83-2.98 (2H, m), 3.27 (2H, t), 3.77-3.87 (2H, m).

### Method U: tert-butyl 2-methyl-2,8-diazaspiro[4.5]decane-8-carboxylate

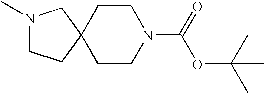

tert-Butyl 2-methyl-1-oxo-2,8-diazaspiro[4.5]decane-8-carboxylate (1.3 g, 4.84 mmol) was taken up in tetrahydrofuran (25 ml) and cooled down to 0° C. Borane 1 M in tetrahydrofuran (15 ml, 15 mmol) was added dropwise. The reaction mixture was then heated to reflux for 18 hours. The reaction was cooled down to 0° C., quenched with methanol (15 ml), and concentrated in vacuo to give the desired compound (1.23 g, quantitative yield). ¹H NMR (CD₃OD, 400 MHz) δ 1.47 (9H, s), 1.50-1.60 (4H, m), 1.74 (2H, t), 2.37 (3H, s), 2.49 (2H, s), 2.66 (2H, t), 3.30-3.50 (4H, m).

### 2-methyl-2,8-diazaspiro[4.5]decane hydrochloride

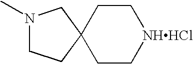

2-methyl-2,8-diazaspiro[4.5]decane hydrochloride was prepared from tert-butyl 2-methyl-2,8-diazaspiro[4.5]decane-8-carboxylate by using method O.

### Example 11

### 4-methyl-4-(pyrrolidin-1-yl)piperidine bis(2,2,2-trifluoroacetate)

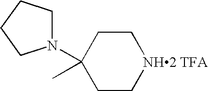

### Method V: 1-tert-butyl 4-ethyl 4-methylpiperidine-1,4-dicarboxylate

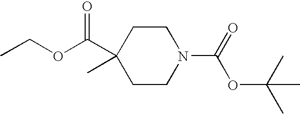

Butyllithium 2.5 M in hexanes (15 ml, 37.5 mmol) was added dropwise over 15 minutes to an ice cold solution of diisopropylamine (5.35 ml, 37.5 mmol) in tetrahydrofuran (200 ml). The solution was stirred at 0° C. for 15 minutes, then cooled to −70° C. A solution of 1-tert-butyl 4-Ethyl piperidine-1,4-dicarboxylate (9.0 g, 35 mmol) in tetrahydrofuran (30 ml) was added dropwise over 15 minutes. The reaction mixture was stirred at −70° C. for a further 1 hour. A solution of methyl iodide (3.2 ml, 52 mmol) in tetrahydrofuran (30 ml) was added dropwise over 15 minutes. The reaction mixture was stirred at −70° C. for a further 2 hours and then allowed to warm up to room temperature overnight. The reaction mixture was partitioned between EtOAc and a saturated solution of ammonium chloride. The organic layer was washed with brine, dried over magnesium sulfate and concentrated in vacuo. The residue was purified on silica gel by flash column chromatography to afford the desired compound (5.7 g, 60% yield). ¹H NMR (CDCl₃, 400 MHz) δ 1.21 (3H, s), 1.28 (3H, t), 1.32-1.42 (2H, m), 1.47 (9H, s), 2.08 (2H, dt), 2.95-3.03 (2H, m), 3.79 (2H, dt), 4.18 (2H, q).

### Method W: 1-(tert-butoxycarbonyl)-4-methylpiperidine-4-carboxylic acid

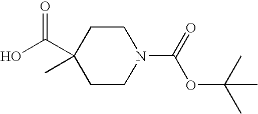

2.0 M sodium hydroxide (20 ml) was added to 1-tert-butyl 4-ethyl 4-methylpiperidine-1,4-dicarboxylate (4 g, 14.76 mmol) in tetrahydrofuran (40 ml) and methanol (5 ml). The solution was stirred at room temperature for 48 hours. The reaction mixture was extracted with ethyl acetate. The aqueous layer was acidified to pH 1-2 with a concentrated solution of HCl. The acid was extracted with EtOAc. The organic layer was washed with brine, dried over magnesium sulfate and concentrated in vacuo to afford the desired compound (2.7 g, 75% yield). ¹H NMR (CDCl₃, 400 MHz) δ 1.29 (3H, s), 1.36-1.48 (2H, m), 1.47 (9H, s), 2.09 (2H, dt), 3.08 (2H, t), 3.79 (2H, br d).

### Method X: tert-butyl 4-isocyanato-4-methylpiperidine-1-carboxylate

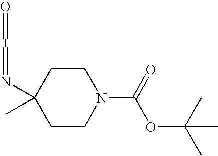

Ethyl chloroformate (1.64 ml, 17 mmol) was added dropwise to a solution of 1-(tert-butoxycarbonyl)-4-methylpiperidine-4-carboxylic acid (2.7 g, 11 mmol) and triethylamine (2.1 ml, (15.5 mmol) in tetrahydrofuran (40 ml) cooled to −15° C. The solution was stirred at −15° C. for 20 minutes. Sodium azide (1.44 g, 22 mmol) was added and the reaction mixture was stirred at −15° C. for a further 1 hour before allowing it to warm up to room temperature. The reaction mixture was partitioned between water and toluene. The organic phase was washed with brine, dried over magnesium sulfate and concentrated in vacuo to a volume of approximately 50 ml. The solution was heated at reflux for 1 hour, after which time, the evolution of nitrogen gas had ceased. The reaction mixture was concentrated in vacuo and purified on silica gel by flash column chromatography to afford the desired compound (560 mg, 22% yield). ¹H NMR (CDCl₃, 400 MHz) δ 1.43 (3H, s), 1.48 (9H, s), 1.49-1.57 (2H, m), 2.67-2.73 (2H, dt), 3.05 (2H, t), 3.96 (2H, m).

### Method Y: tert-butyl 4-amino-4-methylpiperidine-1-carboxylate

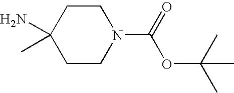

A mixture of potassium hydroxide (400 mg, 7 mmol) and tert-butyl 4-isocyanato-4-methylpiperidine-1-carboxylate (560 mg, 2.3 mmol) in tetrahydrofuran (4 ml) and water (4 ml) was stirred at room temperature for 18 hours. The amine was extracted with ethyl acetate. The organic phase was washed with brine, dried over magnesium sulfate and concentrated in vacuo to afford the desired compound (500 mg, quantitative yield). ¹H NMR (CDCl₃, 400 MHz) δ 1.17 (3H, s), 1.36-1.56 (4H, m), 1.47 (9H, s), 3.38-3.52 (4H, m).

### Method Z: tert-butyl 4-methyl-4-(pyrrolidin-1-yl)piperidine-1-carboxylate

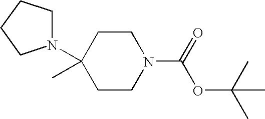

A mixture of tert-butyl 4-amino-4-methylpiperidine-1-carboxylate (300 mg, 1.4 mmol), potassium carbonate (390 mg, 2.8 mmol) and 1,4-dibromobutane (367 mg, 1.7 mmol) in acetonitrile (10 ml) was placed in a sealed tube and stirred at 100° C. for 18 hours. The solid was filtered off and the filtrate was concentrated in vacuo. The residue was purified on silica gel by flash column chromatography to afford the desired compound (102 mg, 27% yield). ¹H NMR (CDCl₃, 400 MHz) δ 1.00 (3H, s), 1.38-1.50 (11H, m), 1.69-1.83 (6H, m), 2.60-2.72 (4H, m), 3.33-3.54 (4H, m).

4-methyl-4-(pyrrolidin-1-yl)piperidine bis(2,2,2-trifluoroacetate)

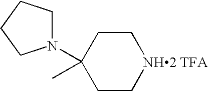

4-methyl-4-(pyrrolidin-1-yl)piperidine bis(2,2,2-trifluoroacetate) was prepared from tert-butyl 4-methyl-4-(pyrrolidin-1-yl)piperidine-1-carboxylate by using method I.

### Example 12

### 4-(2-methoxypropan-2-yl)piperidine

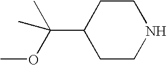

### Method AA: 1-benzyl 4-ethyl piperidine-1,4-dicarboxylate

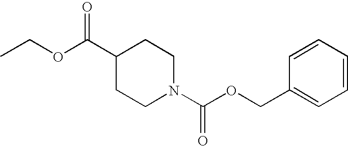

Benzyl chloroformate (4.99 ml, 34.98 mmol) was added dropwise to a solution of ethyl isonipecotate (5.0 g, 31.8 mmol) and triethylamine (5.76 ml, 41.34 mmol) in chloroform (70 ml) at 0° C. After addition, the reaction mixture was stirred at 0° C. for 1 hour, then allowed to warm up to room temperature and stirred for 18 hours. The reaction mixture was washed with 1 M HCl twice and brine. The solution was dried over magnesium sulfate and concentrated in vacuo. The residue was purified on silica gel by flash column chromatography to afford the desired compound as a colourless oil (6.10 g, 66% yield). ¹H NMR (CDCl₃, 400 MHz) δ 1.26 (3H, t), 1.66 (2H, dq), 1.82-1.97 (2H, m), 2.46 (1H, tt), 2.93 (2H, t), 4.02-4.19 (4H, m), 5.13 (2H, s), 7.28-7.39 (5H, m).

### Method BB: benzyl 4-(2-hydroxypropan-2-yl)piperidine-1-carboxylate

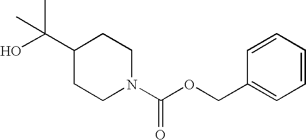

1-benzyl 4-ethyl piperidine-1,4-dicarboxylate (3.0 g, 10.3 mmol) in tetrahydrofuran (15 ml) was added dropwise to a solution of methyl magnesium chloride 3 M in tetrahydrofuran (8.58 ml, 31.8 mmol) in tetrahydrofuran (10 ml) at −78° C. After addition, the reaction mixture was stirred at −78° C. for 1 hour, then allowed to warm up to room temperature and stirred for a further 1 hour. The reaction mixture was quenched by addition of 1 M HCl solution, then extracted with ethyl acetate twice. The combined organics were washed with brine, dried over magnesium sulfate and concentrated in vacuo. The residue was purified on silica gel by flash column chromatography to afford the desired compound as a colourless oil (2.04 g, 71% yield). ¹H NMR (CDCl₃, 400 MHz) δ 1.18 (6H, s), 1.26 (2H, dt), 1.45 (1H, tt), 1.77 (2H, d), 2.72 (2H, t), 4.28 (2H, br s), 5.13 (2H, s), 7.28-7.37 (5H, m).

### Method CC: benzyl 4-(2-methoxypropan-2-yl)piperidine-1-carboxylate

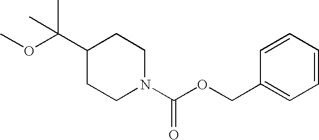

To a suspension of 60% sodium hydride in mineral oil (242 mg, 6.06 mmol) and methyl iodide (0.74 ml, 12.12 mmol) in tetrahydrofuran (10 ml) at room temperature was carefully added portionwise benzyl 4-(2-hydroxypropan-2-yl)piperidine-1-carboxylate (1.12 g, 4.04 mmol) in tetrahydrofuran (10 ml). The reaction mixture was heated to 50° C. for 18 hours. The reaction mixture was cooled down to 0° C. and carefully quenched by addition of a saturated solution of ammonium chloride, then extracted with ethyl acetate twice. The combined organics were washed with brine, dried over magnesium sulfate and concentrated in vacuo. The residue was purified on silica gel by flash column chromatography to afford the desired compound as a colourless oil (634 mg, 54% yield). ¹H NMR (CDCl₃, 400 MHz) δ 1.11 (6H, s), 1.20-1.30 (2H, m), 1.60 (1H, tt), 1.71 (2H, br d), 2.73 (2H, t), 3.20 (3H, s), 4.28 (2H, br s), 5.15 (2H, s), 7.30-7.40 (5H, m).

### Method DD: 4-(2-methoxypropan-2-yl)piperidine

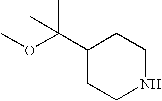

A suspension of benzyl 4-(2-methoxypropan-2-yl)piperidine-1-carboxylate (0.634 g, 2.18 mmol) and 10% palladium on carbon containing 50% of water (0.120 g) in methanol (20 ml) was stirred under an atmosphere of hydrogen for 3 days. The reaction mixture filtered through celite and the filtrate was concentrated in vacuo. The residue was redissolved in methanol (˜4 ml), loaded onto SCX-2 cartridge, washed with methanol and released by washing with 2 N NH3 in methanol. The mixture was concentrated in vacuo to afford the title compound as a yellow oil (0.273 g, 80% yield). ¹H NMR (CDCl₃, 400 MHz) δ 1.11 (6H, s), 1.22-1.50 (2H, m), 1.53-1.75 (2H, m), 1.90 (1H, t), 2.34 (1H, br s), 2.62 (1H, tt), 3.07 (1H, dt), 3.16-3.24 (4H, m).

### Example 13

### N-tert-butyl-N-methylpiperidin-4-amine

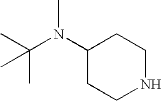

### 1-benzyl-N-tert-butylpiperidin-4-amine

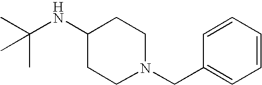

1-benzyl-N-tert-butylpiperidin-4-amine was prepared from N-Benzyl-4-piperidone and tert-Butylamine by using method K. ¹H NMR (CDCl₃, 400 MHz) δ 1.12 (9H, s), 1.35-1.55 (2H, m), 1.60-1.55 (3H, m), 2.04 (2H, dt), 2.54 (1H, m), 2.85 (2H, td), 3.51 (2H, d), 7.25-7.35 (5H, m).

### Method EE: 1-benzyl-N-tert-butyl-N-methylpiperidin-4-amine

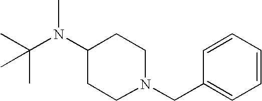

1-benzyl-N-tert-butylpiperidin-4-amine (300 mg, 1.22 mmol), 88% formic acid (0.2 ml, 2.44 mmol) and 37% formaldehyde (0.3 ml, 1.83 mmol) were heated to 55° C. for 12 hours. 8 M potassium hydroxide (0.4 ml) was added in mixture with some brine. The aqueous phase was extracted with ethyl acetate. The organic phase was dried over sodium sulfate and concentrated in vacuo to afford the desired compound as an oil (117 mg, 40% yield). ¹H NMR (CDCl₃, 400 MHz) δ 1.12 (9H, s), 1.57 (2H, br d), 1.77 (2H, dq), 2.00 (2H, dt), 2.27 (3H, s), 2.78 (1H, br t), 2.94 (2H, br td), 3.50 (2H, s), 7.25-7.36 (5H, m).

### N-tert-butyl-N-methylpiperidin-4-amine

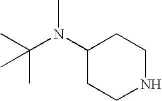

N-tert-butyl-N-methylpiperidin-4-amine was prepared from 1-benzyl-N-tert-butyl-N-methylpiperidin-4-amine by using method Q. ¹H NMR (CDCl₃, 400 MHz) δ 1.36 (9H, s), 1.90-2.05 (4H, m), 2.58 (3H, s), 3.07 (2H, dt), 3.43 (2H, d), 3.68 (1H, m).

The different R¹QH moieties used in the preparation of compounds of formula I wherein Q is a sulfur atom were prepared by three different methodologies starting from commercially available 4-aminothiophenol, bis-(4-aminophenyl)disulfide or from the iodo- derivative. The three strategies are outlined below.

### Example 14

### Method FF: N-(4-mercaptophenyl)cyclopropanecarboxamide

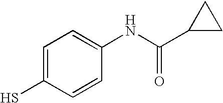

Triethylamine (160.6 ml, 1.14 mol) was added to a solution of 4-aminothiophenol (65.02 g, 520 mmol) in tetrahydrofuran (1 L) cooled down to 0° C. Cyclopropanecarboxylic acid chloride (103.7 ml, 1.14 mol) was added dropwise to keep the temperature below 10° C. The reaction mixture was stirred at 0° C. for 20 minutes then warmed up to room temperature for 1 hour. The solid was filtered off and the filtrate was concentrated in vacuo.

The residue was treated with sodium hydroxide (65.02 g, 1.63 mol) in ethanol (375 ml) and water (625 ml). The reaction mixture was heated to 100° C. for 1 hour, filtered and concentrated under reduced pressure. The residue was diluted with water and filtered through a path of celite. The filtrate was acidified with concentrated hydrochloric acid and the resulting solid was filtered. The solid was dissolved in ethyl acetate (3.75 L) and washed with brine. The organic phase was dried over magnesium sulfate and concentrated in vacuo to afford the title compound (86.3 g, 86% yield). ¹H NMR (DMSO D⁶, 300 MHz) 0.76-0.85 (4H, m), 1.76 (1H, m), 5.19 (1H, s), 7.23 (2H, d), 7.5 (2H, d), 10.18 (1H, s); MS (ES⁺) 194.

Other R¹QH moieties used in the preparation of compounds of formula I of this invention, wherein Q is a sulfur atom, can be prepared via a sequence similar to the one described in example 14 (method FF): 3,3,3-trifluoro-N-(4-mercaptophenyl)propanamide.

### Example 15

### N-(4-mercaptophenyl)propionamide

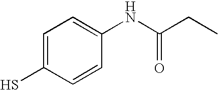

### Method GG: N,N′-(4,4′-disulfanediylbis(4,1-phenylene))dipropionamide

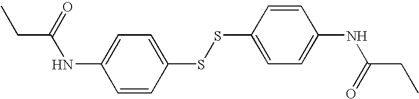

Propionyl chloride (18.3 ml, 0.21 mol) was added to a solution of bis-(4-aminophenyl)disulfide (26 g, 0.10 mmol) and triethylamine (42 ml, 0.30 mol) in dichloromethane (600 ml) cooled down to 0° C. The reaction mixture was stirred at 0° C. for 5 minutes then warmed up to room temperature for 1 hour. During this time, a white precipitate formed. The reaction mixture was concentrated to half of the volume and the white solid was filtered off and washed with a small amount of dichloromethane. The filtrate was again partially concentrated and the remaining white solid was filtered off and washed. The 2 batches of solid were combined (32.4 g, 90% yield). MS (ES⁺) 361, (ES⁻) 359.

### Method HH: N-(4-mercaptophenyl)propionamide

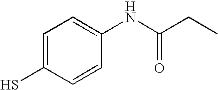

Tris-(2-carboxyethyl)phosphine hydrochloride (TCEP.HCl, 3.66 g, 12.77 mmol) was added to a solution of N,N′-(4,4′-disulfanediylbis(4,1-phenylene))dipropionamide (4 g, 11.1 mmol) and triethylamine (1.67 ml, 11.99 mmol) in a mixture of water (4 ml) and dimethylformamide (25 ml) cooled down to 0° C. The reaction mixture was allowed to warm up to room temperature and was stirred at room temperature for 90 minutes. The reaction mixture was diluted with water (100 ml), causing the precipitation of the desired product. The white solid was isolated by filtration and washed with water. The solid was dissolved in ethyl acetate, dried over magnesium sulfate and concentrated in vacuo to afford the title compound as a white solid (3.13 g, 78% yield). ¹H NMR (DMSO D⁶, 400 MHz) 1.07 (3H, t), 2.29 (2H, q), 5.24 (1H, s), 7.21 (2H, d), 7.48 (2H, d); MS (ES⁺) 182, (ES⁻) 180.

Other R¹QH moieties used in the preparation of compounds of formula I wherein Q is a sulfur atom can be prepared via a sequence similar to the one described in example 15 (methods GG and HH): 4,4,4-trifluoro-N-(4-mercaptophenyl)butanamide, N-(4-mercaptophenyl)-1-(trifluoromethyl)cyclopropanecarboxamide, 2,2-difluoro-N-(4-mercaptophenyl)cyclopropanecarboxamide, 2-cyclopropyl-N-(4-mercaptophenyl)acetamide, 2-cyclopentyl-N-(4-mercaptophenyl)acetamide, 2-chloro-N-(4-mercaptophenyl)benzamide, N-(4-mercaptophenyl)-2-(trifluoromethyl)benzamide.

### Example 16

### N-(2-fluoro-4-mercaptophenyl)cyclopropanecarboxamide

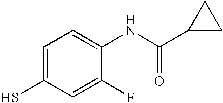

### Method II: N-(2-fluoro-4-iodophenyl)cyclopropanecarboxamide

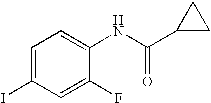

Triethylamine (8 ml, 57.40 mmol) was added to a solution of 2-fluoro-4-iodoaniline (11 g, 46.41 mmol) in tetrahydrofuran (60 ml) cooled down to 0° C. Cyclopropanecarboxylic acid chloride (4.6 ml, 50.60 mmol) was added dropwise to keep the temperature below 10° C. The reaction mixture was stirred at 0° C. for 1 hour then warmed up to room temperature for 30 minutes. The reaction mixture was diluted with ethyl acetate, washed with 1 M hydrochloric acid, a saturated solution of sodium bicarbonate and with brine. The organic phase was dried over magnesium sulfate and concentrated in vacuo. The residue was purified on silica gel by flash column chromatography to afford the title compound as an orange solid (14 g, 99% yield).

### Method JJ: N,N′-(4,4′-disulfanediylbis(2-fluoro-4,1-phenylene))dicyclopropanecarboxamide

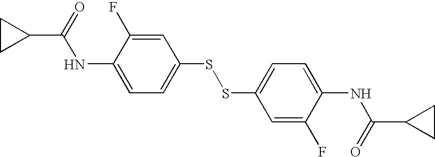

A round bottomed flask was charged with N-(2-fluoro-4-iodophenyl)cyclopropanecarboxamide (15 g, 49.17 mmol), thiourea (7.5 g, 98.53 mmol), nickel on silica (2.5 g) and NMP (100 ml). The mixture was heated at 140° C. for 18 hours. The reaction mixture was allowed to cool down, filtered through celite, diluted with ethyl acetate and washed twice with water and brine. The organic layer was dried over magnesium sulfate, filtered and concentrated in vacuo. The residue was purified by flash column chromatography to afford a mixture of N,N′-(4,4′-disulfanediylbis(2-fluoro-4,1-phenylene))dicyclopropanecarboxamide (2.2 g, 23% yield) and N-(2-fluoro-4-mercaptophenyl)cyclopropanecarboxamide (2.4 g, 11% yield).

### Method KK: N-(2-fluoro-4-mercaptophenyl)cyclopropane-carboxamide

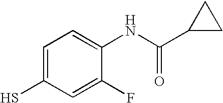

Tris-(2-carboxyethyl)phosphine hydrochloride (TCEP.HCl, 2.3 g, 8.04 mmol) was added to a solution of N,N′-(4,4′-disulfanediylbis(2-fluoro-4,1-phenylene)) dicyclopropanecarboxamide (3.31 g, 7.87 mmol) and triethylamine (1.1 ml, 7.91 mmol) in a mixture of water (2 ml) and dimethylformamide (10 ml). The reaction mixture was stirred at room temperature for 1 hour. The reaction mixture was diluted with water (200 ml). The solid was filtered off, redissolved in ethyl acetate, then washed with brine. The organic layer was dried over magnesium sulfate and concentrated in vacuo to afford the compound.

Other R¹QH moieties used in the preparation of compounds of formula I wherein Q is a sulfur atom can be prepared via a sequence similar to the one described in example 16 (methods II, JJ and KK): N-(3-fluoro-4-mercaptophenyl)cyclopropanecarboxamide.

Table 2 below depicts data for compounds of Table 1. Compound numbers correspond to those compounds depicted in

TABLE 2

| Compound | M + 1 | | Rt |
|---|---|---|---|
| No | (obs) | 1H NMR | (mins) |
| I-1 | 481.57 | (d6-DMSO, 400 MHz) 1.10 (3H, t), 1.38 (6H, m), 2.03 | 2.92 |
| | | (3H, s), 2.10 (1H, m), 2.20-2.50 (3H, m), 3.20-3.52 (3H, | |
| | | m), 3.67 (1H, m), 4.00 (1H, m), 5.40 (1H, m), 5.80 (1H, | |
| | | m), 7.50 (2H, d), 7.71 91H, d), 8.56 (2H, brs), 9.31 (1H, | |
| | | brs), 10.10 (1H, brs) | |
| I-2 | 510.52 | (d6-DMSO, 400 MHz) 0.99 (9 H, br s), 1.10 (3 H, t), | 3.61 |
| | | 1.63-1.60 (2 H, m), 1.91-1.80 (2 H, br m), 2.02 (3 H, br | |
| | | s), 2.34 (2 H, q), 3.32-3.05 (2 H, br m), 3.52 (1H, br m), | |
| | | 4.45-4.37 (1 H, br m), 5.48 (1H, br s), 5.80 (1 H, br s), | |
| | | 7.47 (2 H, d), 7.68 (2H, d), 9.10 (1 H, br s), 10.05 (1 H, | |
| | | s), 11.68 (1 H, br s). | |
| I-3 | 521.0 | (CD₃OD): 1.20-1.30 (3H, t), 1.40-1.50 (4H, m), 1.55- | 3.18 |
| | | 1.60 (1H, s), 2.00-2.30 (7H, m), 2.40-2.45 (2H, qd), | |
| | | 2.50-2.55 (1H, m), 2.65-2.70 (1H, m), 3.30-3.50 (2H, | |
| | | m), 3.65-3.80 (2H, m), 4.00-4.20 (2H, m), 5.50-5.55 (1H, | |
| | | s), 5.70-5.75 (1H, s), 7.60-7.70 (4H, m). | |
| I-4 | 552.53 | (d6-DMSO, 400 MHz) 0.80 (4H, d), 1.25-1.33 (2H, m), | 3.56 |
| | | 1.80-1.89 (3H, m), 2.09 (3H, s), 2.46 masked signal, 2.83 | |
| | | (2H, m), 3.36 masked signal, 3.56 (4H, m), 4.04 (2H, m), | |
| | | 5.85 (1H, s), 6.87 (1H, m), 7.50 (2H, m), 7.75 (2H, m) | |
| I-5 | 511.5 | (d6-DMSO, 400 MHz): 0.82 (4H, m), 1.09 (6H, m), 1.83 | 3.69 |
| | | (1H, m), 1.97 (2H, m), 2.09 (3H, s), 3.44 (1H, m), 3.66 | |
| | | (1H, m), 4.23 (1H, br s), 5.56 (1H, br s), 6.86 (1H, m), | |
| | | 7.52 (2H, m), 7.74 (2H, m), 10.43 (1H, s), 10.78 (1H, s) | |
| I-6 | 469.43 | (d6-DMSO, 400 MHz) 0.81 (4H, m), 1.75-2.02 (4H, m), | 3.29 |
| | | 2.08 (3H, s), 3.25-4.40 (4H, masked signals), 5.51 (1H, | |
| | | s), 6.90 (1H, s), 7.51 (2H, d), 7.71 (2H, d), 10.45 (1H, | |
| | | brs), 10.75 (1H, brs) | |
| I-7 | 509.0 | (d6-DMSO, 400 MHz) 1.10 (3H, t), 1.33 (9H, s), 1.45- | 3.11 |
| | | 1.53 (1H, m), 1.91-2.01 (5H, m), 2.34 (2H, q), 2.89 | |
| | | (2H, t), 4.07 (2H, d), 5.43 (1H, s), 6.08 (1H, brs), 7.47 | |
| | | (2H, d), 7.71 (2H, d), 8.08 (2H, s), 9.29 (1H, s), 10.10 | |
| | | (1H, s), 11.75 (1H, brs) | |
| I-8 | 510.0 | (d6-DMSO, 400 MHz) 0.83-0.81 (4 H, m), 1.49-1.46 | 3.57 |
| | | (1 H, m), 1.85-1.76 (3 H, m), 2.08 (1 H, m), 2.13 (3 H, s), | |
| | | 2.75-2.72 (6 H, m), 3.00 (1 H, m), 3.21 (1 H, br m), 3.40- | |
| | | 3.33 (2 H, m), 4.30-4.27 (1 H, m), 5.93 (1 H, s), 6.94 | |
| | | (1 H, s), 7.52 (2 H, d, J 8.5), 7.74 (2 H, d, J 8.5), 9.99 | |
| | | (1 H, br s), 10.52 (1 H, s), 11.12 (1 H, br s). | |
| I-9 | 453.0 | (d6-DMSO, 400 MHz) 1.10 (3 H, t, J 7.5), 2.01 (3 H, s), | 2.73 |
| | | 2.13 (1 H, m), 2.38-2.26 (3 H, m), 2.62 (3 H, s), 3.34- | |
| | | 3.32 (1 H, m), 3.49-3.42 (2 H, m), 3.63-3.59 (1 H, m), | |
| | | 3.82 (1 H, m), 5.41 (1 H, m), 5.76 (1 H, br s), 7.48 (2 H, | |
| | | d, J 8.6), 7.71 (2 H, d, J 8.6), 8.65-8.64 (2 H, m), 9.30 | |
| | | (1 H, s), 10.10 (1 H, s). | |
| I-10 | 496.53 | (d6-DMSO, 400 MHz) 0.95 (9 H, s), 1.10 (3 H, t), 1.70 | 3.42 |
| | | (1 H, m), 1.98 (1 H, m), 2.03 (3 H, s), 2.34 (2 H, q), | |
| | | 3.49.3.19 (4 H, masked signals), 5.48 (1 H, s), 5.75 (1 H, | |
| | | br s), 7.48 (2 H, d), 7.70 (2 H, d), 9.18 (1 H, br s), 10.04 | |
| | | (1 H, s). | |
| I-11 | 454.48 | (d6-DMSO, 400 MHz) 1.10 (3 H, t), 1.31 (3 H, s), 1.84 | 3.12 |
| | | (2 H, br m), 2.01 (3 H, s), 2.34 (2 H, q), 2.50 (2 H, | |
| | | masked signal), 3.12 (2 H, d), 4.79 (1 H, br s), 5.42 (1 H, | |
| | | s), 5.72 (1 H, br s), 7.48 (2 H, d), 7.69 (2 H, d), 9.14 | |
| | | (1 H, s), 10.07 (1 H, s), 11.70 (1 H, br s). | |
| I-12 | 469.37 | (d6-DMSO, 400 MHz) 0.81 (4H, m), 1.75-2.02 (4H, m), | 3.31 |
| | | 2.08 (3H, s), 3.25-3.40 (2H, m), 4.32 (1H, m), 4.98 (1H, | |
| | | m), 5.51 (1H, s), 6.90 (1H, s), 7.51 (2H, d), 7.71 (2H, d), | |
| | | 10.45 (1H, brs), 10.75 (1H, brs) | |
| I-13 | 510.0 | (d6-DMSO, 400 MHz) 0.83-0.81 (4 H, m), 1.49-1.46 (1 | 3.58 |
| | | H, m), 1.85-1.76 (3 H, m), 2.08 (1 H, m), 2.13 (3 H, s), | |
| | | 2.75-2.72 (6 H, m), 3.00 (1 H, m), 3.21 (1 H, br m), 3.40- | |
| | | 3.33 (2 H, m), 4.30-4.27 (1 H, m), 5.93 (1 H, s), 6.94 | |
| | | (1 H, s), 7.52 (2 H, d, J 8.5), 7.74 (2 H, d, J 8.5), 9.99 | |
| | | (1 H, brs), 10.52 (1 H, s), 11.12 (1 H, br s). | |
| I-14 | 483.49 | (d6-DMSO, 400 MHz): 0.97 (4H, m), 1.96 (1H, m), | 3.4 |
| | | 2.06-2.23 (5H, m), 3.40 (5H, m), 5.74 (1H, s), 7.06 (1H, | |
| | | s), 7.66 (2H, m), 7.89 (2H, m), 10.59 (1H, s), 11.20 (1H, | |
| | | brs) | |
| I-15 | 496.47 | (d6-DMSO, 400 MHz) 1.10 (3H, t), 1.57 (4H, m), 2.01 | 3.31 |
| | | (3H, s), 2.35 (2H, q), 3.47 (4H, m), 3.91 (4H, s), 5.43 | |
| | | (1H, s), 6.07 (1H, s), 7.48 (2H, d), 7.76 (2H, d), 9.21 | |
| | | (1H, s), 10.14 (1H, s), 11.70 (1H, s) | |
| I-16 | 463.54 | (d6-DMSO, 400 MHz): 1.10 (3H, m), 1.65 (2H, m), 1.86 | 3.37 |
| | | (2H, m), 2.01 (3H, s), 2.36 (2H, m), 3.10 (1H, m), 3.21 | |
| | | (2H, m), 3.63 (2H, m), 5.43 (1H, s), 6.65 (1H br s), 7.47 | |
| | | (2H, m), 7.69 (2H, m), 9.25 (1H, s), 10.08 (1H, s), 11.72 | |
| | | (1H, br s) | |
| I-17 | 452.36 | (d6-DMSO, 400 MHz) 1.09 (3H, t), 2.03 (3H, s), 2.39 | 3.24 |
| | | (6H, m), 3.70 (4H, t), 5.52 (1H, br s), 6.14 (1H, br s), | |
| | | 7.48 (2H, d), 7.71 (2H, d), 9.37 (1H, s), 10.08 (1H, s) | |
| I-18 | 442.4 | (d6-DMSO, 400 MHz) 1.14 (3H, t), 2.03 (3H, s), 2.2-2.28 | 3.32 |
| | | (2H, m), 2.42 (2H, q), 3.27-3.32 (1H, m), 3.45-3.62 | |
| | | (3H, m), 5.32 (0.5H, s), 5.43-5.48 (1.5H, m), 5.8 91H, brs), | |
| | | 7.52 (2H, d), 7.72 (2H, d), 9.25 (1H, brs), 10.09 (1H, s), | |
| | | 11.69 (1H, s) | |
| I-19 | 479.5 | (d6-DMSO, 400 MHz) 1.0-1.2 (3H, m, alk), 1.2-1.8 (5H, | 2.87 |
| | | m, alk), 1.9-2.7 (6H, m, alk), 2.8 (H, m, alk), 3.0-3.7 | |
| | | (5H, m, alk), 5.4 (H, brs, ar), 5.75 (H, brs, ar), 7.4-7.6 | |
| | | (2H, m, ar), 7.6-7.8 (2H, m, ar), 9.1 (H, s, NH), 10.05 (H, | |
| | | s, NH) and 11.7 (H, brs, NH) | |
| I-20 | 468.4 | (d6-DMSO, 400 MHz) 1.14 (3H, t), 2.03 (3H, s), 2.2-2.28 | 3.4 |
| | | (2H, m), 2.42 (2H, q), 3.27-3.32 (1H, m), 3.45-3.62 | |
| | | (3H, m), 5.32 (0.5H, s), 5.43-5.48 (1.5H, m), 5.8 91H, brs), | |
| | | 7.52 (2H, d), 7.72 (2H, d), 9.25 (1H, brs), 10.09 (1H, s), | |
| | | 11.69 (1H, s) | |
| I-21 | 468.0 | (d6-DMSO, 400 MHz) 1.08-1.18 (6H, m), 1.30-1.45 | 3.21 |
| | | (4H, m), 2.03 (3H, s), 2.34-2.38 (2H, m), 3.20 (2H, t), | |
| | | 3.64 (2H, d), 5.47 (1H, s), 6.05 (1H, brs), 7.48 (2H, d), | |
| | | 7.69 (2H, d), 9.28 (1H, s), 10.09 (1H, s), 12.00 (1H, brs) | |
| I-22 | 453.45 | (d6-DMSO, 400 MHz) 1.10 (3 H, t, J 7.5)), 2.01 (3 H, s), | 2.88 |
| | | 2.13-2.12 (1 H, m), 2.38-2.27 (3 H, m), 2.63-2.61 (3 H, | |
| | | m), 3.32 (1 H, m), 3.46-3.42 (2 H, m), 3.61-3.59 (1 H, | |
| | | m), 3.82 (1 H, m), 5.41 (1 H, s), 5.76 (1 H, br s), 7.48 | |
| | | (2 H, d, J 8.6), 7.71 (2 H, d, J 8.6), 8.68-8.66 (2 H, m), | |
| | | 9.32 (1 H, s), 10.10 (1 H, s). | |
| I-23 | 494.0 | (d6-DMSO, 400 MHz) 0.83 (9H, s), 0.98-1.08 (2H, partly | 4.1 |
| | | obscured m), 1.10 (3H, t), 1.18-1.25 (1H, m), 1.65 (2H, | |
| | | brd), 2.01 (3H, brs), 2.35 (2H, q), 2.65 (2H, brt), 4.13 | |
| | | (2H, brd), 5.43 (1H, brs), 6.05 (1H, vbrs), 7.47 (2H, d), | |
| | | 7.70 (2H, d), 9.16 (1H, brs), 10.08 (1H, s), 11.69 (1H, brs). | |
| I-24 | 467.0 | (d6-DMSO, 400 MHz) 1.09 (3 H, t, J 7.5), 1.75-1.73 (1 H, | 465.0 |
| | | m), 2.01 (3 H, s), 2.16-2.07 (2 H, masked signal), 2.16 | |
| | | (6 H, s), 2.33 (2 H, q, J 7.5), 2.67 (1 H, br m), 2.96-2.92 | |
| | | (1 H, m), 3.19-3.18 (1 H, m), 3.65-3.50 (1 H, br m), 5.43 | |
| | | (1 H, s), 5.78 (1 H, br s), 7.47 (2 H, d, J 8.6), 7.69 (2 H, | |
| | | d, J 8.6), 9.13 (1 H, s), 10.06 (1 H, s). | |
| I-25 | 496.0 | (d6-DMSO, 400 MHz) 0.83-0.81 (4 H, m), 1.50 (1 H, | 3.29 |
| | | m), 1.67 (1 H, m), 1.84-1.79 (2 H, m), 2.07 (2 H, m), | |
| | | 2.11 (3 H, s), 3.17-3.15 (2 H, m), 3.44-3.28 (1 H, m), | |
| | | 3.55-3.52 (1 H, m), 4.08-3.68 (1 H, masked signal), 5.90 | |
| | | (1 H, s), 6.90 (1 H, s), 7.51 (2 H, d, J 8.6), 7.74 (2 H, d, J | |
| | | 8.6), 8.68-8.57 (2 H, m), 10.49 (1 H, s), 10.98 (1 H, br s). | |
| I-26 | 528.24 | (d6-DMSO, 400 MHz) 1.69 (3H, s), 2.06 (6H, m), 3.53 | 3.53 |
| | | (2H, q), 3.76 (2H, t), 4.79 (1H, s), 6.12 (1H, br s), 7.55 | |
| | | (2H, d), 7.74 (2H, d), 9.31 (1H, s), 10.66 (1H, s) | |
| I-27 | 514.19 | (d6-DMSO, 400 MHz) 2.01 (3H, s), 3.50 (6H, m), 3.69 | 3.49 |
| | | (2H, m), 5.45 (1H, s), 5.80 (1H, br s), 7.55 (2H, d), 7.75 | |
| | | (2H, d), 9.33 (1H, s), 10.50 (1H, s) | |
| I-28 | 481.46 | (d6-DMSO, 400 MHz): 1.11 (3H, t), 1.24 (6H, d), 2.01 | 2.89 |
| | | (3H, s), 2.10 (1H, m), 2.34 (3H, m), 2.54 (4H, m), 3.65 | |
| | | (1H, s), 3.97 (1H, s), 5.43 (1H, s), 5.78 (1H, br s), 7.49 | |
| | | (2H, d), 7.70 (2H, d), 8.56 (2H, br s), 9.32 (1H, s), 10.10 | |
| | | (1H, s) | |
| I-29 | 496.51 | (d6-DMSO, 400 MHz): 0.82 (4H, m), 1.80 (2H, m), 2.08 | 3.44 |
| | | (4H, m), 2.17 (6H, s), 3.00 (1H, m), 3.25 (1H, m), 3.35 | |
| | | masked signal, 5.56 (1H, s), 6.87 (1H, m), 7.52 (2H, m), | |
| | | 7.72 (2H, m) | |
| I-30 | 496.45 | (d6-DMSO, 400 MHz): 0.81 (4H, d), 1.79 (2H, s), 1.82 | 3.5 |
| | | (1H, m), 2.09 (3H, s), 2.17 (5H, s), 2.54 (1H, s), 3.02 | |
| | | (6H, s), 5.55 (1H, s), 6.86 (1H, s), 7.52 (2H, d), 7.73 (2H, | |
| | | d), 10.42 (1H, s) | |
| I-31 | 547.0 | (CD₃OD, 400 MHz): 0.80-0.87 (2H, m), 0.90-0.95 (2H, | 3.08 |
| | | m), 1.02-1.07 (6H, d), 1.45-1.50 (4H, m), 1.65-1.70 (2H, | |
| | | t), 1.75-1.80 (1H, m), 2.10 (3H, s), 2.30-2.40 (1H, m), | |
| | | 2.50 (2H, s), 2.65-2.70 (2H, t), 3.25-3.37 (4H, m), 3.45- | |
| | | 2.50 (2H, m), 5.50-5.60 (1H, br s), 5.80-5.90 (1H, br s), | |
| | | 7.45-7.50 (2H, m), 7.62-7.65 (2H, m). | |
| I-32 | — | (CD₃OD, 400 MHz): 0.95-1.05 (4H, m), 1.65-1.80 (4H, | — |
| | | m), 1.90-2.05 (2H, m), 2.10-2.20 (4H, m), 2.90-2.95 (4H, | |
| | | m), 3.20-3.25 (1H, m), 3.50-3.75 (6H, m), 5.67 (1H, s), | |
| | | 5.80 (1H, s), 7.50-7.60 (2H, d*d), 7.85-7.90 (1H, t). | |
| I-33 | 535.0 | (CD₃OD, 400 MHz): 1.20-1.30 (5H, m), 1.45 (3H, s), | 3.09 |
| | | 1.55 (3H, s), 1.60-1.70 (2H, m), 1.95-2.10 (4H, m), 2.20 | |
| | | (3H, s), 2.25-2.30 (1H, m), 2.40-2.45 (2H, qd), 2.95-3.05 | |
| | | (2H, m), 3.30-3.40 (2H, m), 3.65-3.75 (2H, m), 4.35-4.65 | |
| | | (2H, m), 5.65 (1H, s), 5.80 (1H, s), 7.60-7.70 (4H, qd). | |
| I-34 | 521.0 | (CD₃OD, 400 MHz): 1.20-1.40 (5H, m), 1.55 (3H, s), | 2.96 |
| | | 1.90-2.15 (6H, m), 2.25 (3H, s), 2.40-2.45 (2H, qd), 3.2- | |
| | | 3.5 (8H, m), 4.3-4.4 (2H, m), 5.70 (1H, s), 5.80 (1H, s), | |
| | | 7.60-7.65 (2H, d), 7.70-7.75 (2H, d). | |
| I-35 | 524.6 | (d6-DMSO, 400 MHz) 2.06 (3H, s), 2.16-2.23 (2H, m), | 3.58 |
| | | 3.42-3.55 (3H, m), 5.33 (0.5H, s), 5.45 (1H, s), 5.75 | |
| | | (1H, vbrs), 7.46-7.62 (6H, m), 7.84 (2H, d), 9.24 (1H, brs), | |
| | | 10.74 (1H, s), 11.69 (1H, brs) | |
| I-36 | 482.46 | (d6-DMSO, 400 MHz) 0.83-0.81 (4 H, m), 1.85-1.79 (1 | 3.18 |
| | | H, m), 2.09 (3 H, s), 2.16 (1 H, m), 2.33 (1 H, m), 2.68- | |
| | | 2.62 (3 H, m), 3.37 (1 H, m), 3.55-3.48 (2 H, br m), 3.66 | |
| | | (1 H, m), 3.84 (1 H, m), 5.61 (1 H, s), 6.88 (1 H, s), 7.51 | |
| | | (2 H, d, J 8.6), 7.74 (2 H, d, J 8.6), 8.69 (2 H, br m), | |
| | | 10.46 (1 H, s), 10.91 (1 H, br s). | |
| I-37 | 522.56 | (d6-DMSO, 400 MHz) 0.86 (4H, m), 1.70-2.10 (7H, m), | 3.36 |
| | | 2.11-2.50 (3H, m), 3.05-4.15 (9H, m), 5.60 (1H, s), 6.91 | |
| | | (1H, s), 7.55 (2H, d), 7.80 (2H, d), 10.00 (1H, brs), 10.45 | |
| | | (1H, s), 10.98 (1H, brs). | |
| I-38 | 536.63 | (d6-DMSO, 400 MHz): 0.82 (4H, m), 1.48 (2H, m), 1.82 | 3.32 |
| | | (3H, m), 2.01 (2H, m), 2.10 (5H, m), 2.86 (2H, m), 3.09- | |
| | | 3.15 (2H, m), 3.40 (1H, m), 3.53 (2H, m), 4.20 (2H, m), | |
| | | 5.89 (1H, s), 6.89 (1H, s), 7.53 (2H, d), 7.75 (2H, d), | |
| | | 9.49 (1H, br s), 10.46 (1H, s), 10.90 (1H, br s) | |
| I-39 | 467.0 | (d6-DMSO, 400 MHz) 1.10 (3 H, t, J 7.5), 1.20 (3 H, t, J | 2.79 |
| | | 7.2), 2.01 (3 H, s), 2.13 (1 H, m), 2.38-2.27 (3 H, m), | |
| | | 3.04-3.00 (2 H, m), 3.33-3.31 (1 H, m), 3.48-3.46 (2 H, | |
| | | m), 3.64-3.60 (1 H, m), 3.87 (1 H, m), 5.42 (1 H, s), 5.77 | |
| | | (1 H, br s), 5.77 (1 H, br s), 7.48 (2 H, d, J 8.6), 7.71 (2 | |
| | | H, d, J 8.6), 8.59 (2 H, m), 9.33 (1 H, s), 10.1 1 (1 H, s). | |
| I-40 | 523.7 | (d6-DMSO, 400 MHz) 1.09 (3H, t), 1.22-1.30 (2H, m), | 3.29 |
| | | 1.75-1.80 (2H, m), 2.01 (3H, s), 2.37-2.47 (2H, m), 2.70- | |
| | | 2.80 (2H, m), 3.53-3.58 (4H, m), 4.02-4.08 (2H, m), 5.44 | |
| | | (1H, brs), 7.47 (2H, d), 7.70 (2H, d), 9.18 (1H, brs), 10.07 | |
| | | (1H, brs), 11-69 (1H, brs) | |
| I-41 | 507.0 | (d6-DMSO, 400 MHz) 1.05-1.15(3H, t, Et), 1.4-1.5 (2H, | 2.91 |
| | | m, alk), 1.75-1.9 (2H, m, alk), 1.9-2.1 (7H, m, alk), 2.3- | |
| | | 2.4 (2H, q, Et), 2.7-2.9 (2H, m, alk), 3.0-3.15 (2H, m, | |
| | | alk), 3.35 (H, m, alk), 3.5-3.6 (2H, m, alk), 4.1-4.2 (2H, | |
| | | m, alk), 5.4 (H, s, ar), 6.1 (H, s, ar0, 7.45 (2H, d, ar), 7.7 | |
| | | (2H, d, ar), 9.3 (H, s, NH), 9.5 (H, brs, NH) and 10.1 (H, | |
| | | s, NH). | |
| I-42 | 496.0 | (d6-DMSO, 400 MHz) 1.03 (6 H, s), 1.11-1.08 (5 H, m), | 3.34 |
| | | 1.38 (1 H, m), 1.69 (2 H, d), 2.01 (3 H, s), 2.34 (2 H, q), | |
| | | 2.68-2.65 (2 H, m), 4.14-4.12 (3 H, m), 5.44 (1 H, br s), | |
| | | 6.07 (1 H, br s), 7.47 (2 H, d), 7.69 (2 H, d), 9.15 (1 H, br | |
| | | s), 10.07 (1H, s), 11.70 (1H, br s). | |
| I-43 | 525.7 | (d6-DMSO, 400 MHz) 1.14 93H, t), 1.25-1.35 (2H, m), | 3.38 |
| | | 1.86-1.94 (2H, m), 2.06 (3H, s), 2.25-2.3 (1H, m), 2.42 | |
| | | (2H, q), 2.65-2.8 (1H, m), 2.85-2.97 (3H, m), 3.92-3.96 | |
| | | (2H, m), 5.13-5.15 (0.5H, m), 5.31-5.34 (0.5H, m), 5.5 | |
| | | (1H, s), 6.15 (1H, vbrs), 7.52 (2H, d), 7.78 (2H, d), 9.25 | |
| | | (1H, brs), 10.14 (1H, s), 11.65 (1H, brs) | |
| I-44 | 482.0 | (d6-DMSO, 400 MHz) 0.83 (3H, t), 1.09 (3H, t), 1.23- | 3.37 |
| | | 1.44 (4H, m), 2.01 (3H, s), 2.33 (2H, q), 3.10-3.18 (2H, | |
| | | m), 3.73 (2H, d), 4.17 (1H, s), 5.44 (1H, s), 6.11 (1H, | |
| | | brs), 7.47 (2H, d), 7.73 (2H, d), 9.14 (1H, s), 10.13 (1H, | |
| | | s), 11.70 (1H, brs) | |
| I-45 | 510.55 | (d6-DMSO, 400 MHz) 1.03 (6 H, s), 1.15-1.08 (5 H, m), | 3.69 |
| | | 1.63 (3 H, m), 2.02 (3 H, s), 2.34 (2 H, q), 2.67 (2 H, t), | |
| | | 3.08 (3 H, s), 4.13 (2 H, d), 5.46 (1 H, s), 5.77 (1 H, s), | |
| | | 6.05 (1H, br s), 7.48 (2 H, d), 7.70 (2 H, d), 9.28 (1 H, s), | |
| | | 10.09 (1 H, s). | |
| I-46 | 467.48 | (d6-DMSO, 400 MHz): 1.09 (3H, t), 2.00 (3H, s), 2.37 | 3.06 |
| | | (2H, q), 2.82 (6H, s), 3.29 (1H, m), 3.46 (2H, m), 3.78 | |
| | | (2H, m), 3.99 (2H, m), 5.40 (1H, s), 5.70 (1H, br s), 7.49 | |
| | | (2H, d), 7.72 (2H, d), 9.31 (1H, s), 9.82 (1H, s), 10.09 | |
| | | (1H, s) | |
| I-47 | 523.0 | (d6-DMSO, 400 MHz) 1.10 (3H, t), 1.37 (9H, s), 1.58- | 3.35 |
| | | 1.87 (4H, m), 2.34 (2H, q), 2.90-2.98 (2H, m), 3.58- | |
| | | 3.66 (1H, m), 3.86-3.92 (1H, m), 4.10 (1H, d), 4.20 | |
| | | (1H, d), 5.44 (1H, s), 6.04 (1H, brs), 7.48 (2H, d), 7.70 | |
| | | (2H, d), 8.26 (0.5H, brs), 8.58 (1H, s), 9.28 (1H, s), 10.10 | |
| | | (1H, s), 11.72 (1H, brs). | |
| I-48 | 493.0 | (CD3OD): 1.20-1.30 (3H, t), 2.00-2.25 (7H, m), 2.30- | 3.17 |
| | | 2.40 (1H, br s), 2.45-2.60 (3H, m), 3.20-3.30 (2H, m), | |
| | | 3.50-3.60 (1H, m), 3.70-3.80 (3H, m), 4.00-4.20 (2H, m), | |
| | | 5.55 (1H, s), 5.80 (1H, s), 7.60-7.65 (2H, d), 7.80-7.85 | |
| | | (2H, d). | |
| I-49 | 524.33 | (d6-DMSO, 400 MHz) 1.05-1.1 (3H, t, CH3), 1.4-1.55 | 3.11 |
| | | (2H, m, alk), 1.8-1.95 (2H, m, alk), 1.95-2.1 (2H, q, | |
| | | CH2), 2.8-2.9 (2H, m, alk), 3.0-3.15 (22H, m, alk), 3.4 | |
| | | (H, m, alk), 3.45-3.6 (2H, m, alk), 4.1-4.25 (2H, m, alk), | |
| | | 5.9 (H, s, ar), 6.9 (H, s, ar), 7.5-7.55 (2H, d, ar), 7.75-7.8 | |
| | | (2H, d, ar), 9.6 (H, brs, NH), 10.15 (H, s, NH) and 10.95 | |
| | | (H, brs, NH). | |
| I-50 | 481.61 | (d6-DMSO, 400 MHz) 1.1-1.15 (3H, t, CH3), 1.25-1.35 | 3.17 |
| | | (3H, m, CH3), 2.05 (3H, s, CH3), 2.3 (H, m, alk), 2.35- | |
| | | 2.45 (3H, m, alk), 2.75(2H, m, alk), 3.0-3.35 (4H, m, | |
| | | alk), 3.5-3.6 (2H, m, alk), 3.8 (H, m, alk), 4.0 (H, m, alk), | |
| | | 5.5 (H, m, ar), 6.75 (H, m, ar), 7.5 (2H, d, ar), 7.75 (2H, | |
| | | d, ar), 9.65 (H, alk, NH), 10.2 (H, s, NH) and 10.7 (H, | |
| | | brs, alk) | |
| I-51 | 545.0 | (CD₃OD, 400 MHz): 0.55-0.60 (2H, m), 0.85-0.95 (4H, | 3.23 |
| | | m), 0.95-1.00 (2H, m), 1.50-1.55 (4H, m), 1.70-1.80 (4H, | |
| | | m), 2.35 (3H, s), 2.48 (2H, s), 2.60-2.67 (2H, t), 3.35- | |
| | | 3.50 (5H, m), 5.50-5.65 (1H, br s), 5.90-6.00 (1H, br s), | |
| | | 7.40-7.50 (2H, m), 7.62-7.67 (2H, m). | |
| I-52 | 482.0 | (d6-DMSO, 400 MHz) 0.83-0.81 (4 H, m), 1.82-1.80 (1 H, m), | 3.15 |
| | | 2.09 (3 H, s), 2.16-2.14 (1 H, m), 2.39-2.30 (1 H, m), | |
| | | 2.65 (3 H, s), 4.24-3.28 (5 H, masked signal), 5.61 (1 H, | |
| | | s), 6.88 (1 H, s), 7.51 (2 H, d, J 8.6), 7.74 (2 H, d, J | |
| | | 8.6), 8.66 (2 H, m), 10.45 (1 H, s), 10.90 (1 H, br s). | |
| I-53 | 507.6 | (d6-DMSO, 400 MHz) 1.09 (3H, t), 1.5-1.6 (3H, m), 1.78- | 3.03 |
| | | 1.85 (1H, m), 2.03 (3H, s), 2.34 (2H, q), 2.84 (3H, s), 3.1- | |
| | | 3.17 (1H, m), 3.3-3.55 (7H, m), 5.45 (1H, s), 6.05 (1H, s), | |
| | | 7.47 (2H, d), 7.70 (2H, d), 9.27 (1H, s), 9.80 (1H, brs), | |
| | | 10.10 (1H, brs), | |
| I-54 | — | (CD₃OD, 400 MHz): 0.95-1.05 (4H, m), 1.60-1.80 (4H, | — |
| | | m), 1.90-2.05 (1H, m), 2.10-2.20 (4H, m), 2.90-2.95 (4H, | |
| | | m), 3.20-3.25 (1H, m), 3.45-3.75 (6H, m), 5.70 (1H, s), | |
| | | 5.80 (1H, s), 7.30-7.35 (2H, d), 7.57-7.62 (1H, t), 7.63- | |
| | | 7.68 (1H, d). | |
| I-55 | 493.52 | (d6-DMSO, 400 MHz) 1.1-1.5 (3H, m, alk), 1.75-2.15 | 3.09 |
| | | (7H, m, alk), 2.3 (H, m, alk), 2.35-2.45 (2H, m, alk), | |
| | | 3.05-3.25 (4H, m, alk), 3.3 (H, m, alk), 3.4-3.65 (4H, m, | |
| | | alk), 3.8 (H, m, alk), 4.0 (H, m, alk), 5.5 (H. s, ar), 5.8 | |
| | | (H, s, ar), 7.5 (2H, d, ar), 7.75 (2H, d, ar), 9.4 (H, s, NH), | |
| | | 10.3 (H, s, NH) and 10.9 (H, brs, NH). | |
| I-56 | 533.62 | (d6-DMSO, 400 MHz) 0.5-0.6 (2H, m, alk), 0.8-0.9 (2H, | 3.03 |
| | | m, alk), 1.05-1.15 (3H, t, CH3), 1.45-1.6 (2H, m, alk), | |
| | | 1.75 (H, m, alk), 1.85 (H, m, alk), 1.95-2.1 (2H, m, alk), | |
| | | 2.35-2.4 (2H, m, alk), 2.75-2.85 (2H, m, alk), 3.0-3.15 | |
| | | (2H, m, alk), 3.35 (H, m, alk), 3.5 (2H, m, alk), 4.15 (2H, | |
| | | m, alk), 5.5 (H, s, ar), 6.15 (H, brs, ar), 7.5-7.55 (2H, d, | |
| | | ar), 7.7-7.75 (2H, d, ar), 9.5 (H, s, NH), 10.1 (H, s, NH) | |
| | | and 10.25 (H, brs, NH). | |
| I-57 | 535.6 | (d6-DMSO, 400 MHz) 0.9-0.98 (6H, m), 1.1 (3H, t), 1.25- | 3.06 |
| | | 1.3 (4H, m), 1.7-1.9 (2H, m), 2.03 (3H, s), 2.34 (2H, q), | |
| | | 2.7-2.8 (2H, m), 3.17-3.27 (1H, m), 3.98-4.12 (2H, m), | |
| | | 5.43 (1H, s), 6.05 (1H, brs), 7.47 (2H, d), 7.70 (2H, d), 9.18 | |
| | | (1H, brs), 10.07 (1H, s), 11.69 (1H, s) | |
| I-58 | 496.0 | (d6-DMSO, 400 MHz) 0.83 (6H, d), 1.09 (3H, t), 1.29- | 3.49 |
| | | 1.52 (5H, m), 2.00 (3H, s), 2.33 (2H, q), 3.05 (2H, t), | |
| | | 3.84 (2H, d), 4.07 (1H, s), 5.44 (1H, s), 6.10 (1H, brs), | |
| | | 7.47 (2H, d), 7.69 (2H, d), 9.16 (1H, s), 10.07 (1H, s), | |
| | | 11.71 (1H, brs) | |
| I-59 | 508.49 | (d6-DMSO, 400 MHz) 0.81 (4 H, d, J 6.1), 1.40 (1 H, br | 3.19 |
| | | m), 1.70-1.51 (3H, br m), 1.84-1.78 (1 H, m), 2.08 (3 H, | |
| | | s), 2.34-2.20 (1 H, m), 2.55-2.50 (2 H, masked signal), | |
| | | 2.85-2.82 (1 H, m), 3.17-3.01 (1 H, m), 3.50-3.25 (3 H, | |
| | | masked signal), 5.53-5.51 (1 H, m), 6.85 (1 H, s), 7.51 | |
| | | (2 H, d, J 8.6), 7.72 (2 H, d, J 8.6), 10.43 (1 H, s), 10.76 | |
| | | (1 H, br s). | |
| I-60 | 507.53 | (d6-DMSO, 400 MHz): 0.63 (1H, m), 1.09 (3H, m), 1.28 | 3.0 |
| | | (6H, m), 1.81 (1H, m), 2.39 (2H, m), 2.90-3.06 (8H, m), | |
| | | 3.31-3.56 (3H, m), 3.73 (1H, m), 5.41 (1H, s), 5.77 (1H, | |
| | | br s), 7.49 (2H, m), 7.72 (2H, m), 9.68 (1H, m), 10.18 | |
| | | (1H, s), 10.73 (1H, s) | |
| I-61 | 510.0 | (d6-DMSO, 400 MHz) 0.84 (9H, s), 1.09 (3H, t), 1.45 | 3.63 |
| | | (4H, brs), 2.01 (3H, s), 2.34 (2H, q), 2.98-3.05 (2H, m), | |
| | | 3.87-3.90 (2H, m), 5.44 (1H, s), 6.15 (1H, brs), 7.47 | |
| | | (2H, d), 7.69 (2H, d), 9.14 (1H, s), 10.07 (1H, s), 11.70 | |
| | | (1H, s) | |
| I-62 | 519.57 | (d6-DMSO, 400 MHz) 0.5-0.55 (2H, m, alK), 0.8-0.85 | 3.18 |
| | | (2H, m, alk), 1.05-1.15 (3H, t, CH3), 1.65 (H, m, alk), | |
| | | 1.8-1.95 (2H, m, alk), 2.0-2.1 (2H, m, alk), 2.25 (H, m, | |
| | | alk), 2.3-2.4 (2H, q CH2), 3.1-3.25 (4H, m, alk), 3.3 (H, | |
| | | m, alk), 3.75 (2H, m, alk), 3.95 (2H, m, alk), 5.5 (H, s, | |
| | | ar), 5.85 (H, brs, ar), 7.45-7.5 (2H, d, ar), 7.7-7.75 (2H, d, | |
| | | ar), 9.35 (H, s, NH), 10.1 (H, s, NH) and 10.4 (H, s, NH). | |
| I-63 | 579.3 | (d6-DMSO, 400 MHz) 1.1-1.2 (2H,m), 1.65-1.72 | 3.34 |
| | | (1H, m), 1.85 (3H, s), 2.2-2.3 (1H, m), 2.6-2.75(4H, m), 3.4 | |
| | | (2H, q), 3.75-3.8 (2H, m), 5.15 (0.5H, m), 5.23-5.28 | |
| | | (1.5H, m), 5.91 (1H, vbrs), 6.38 (1H, brs), 7.38 (2H, d), 7.5 | |
| | | (2H, d), 9.16 (1H, brs), 10.33 (1H, brs), 11.7 (1H, brs) | |
| I-64 | 616.68 | (CDCl₃) 1.51 (6H, m), 2.05 (2H, d), 2.20 (3H, s), 2.84 | 3.36 |
| | | (3H, s), 3.02 (6H, m), 3.33 (6H, m), 5.64 (1H, s), 5.78 | |
| | | (1H, s), 7.58 (2H, d), 7.78 (2H, d) | |
| I-65 | 587.0 | (d6-DMSO, 400 MHz) 1.35-1.39 (2H, m), 1.41-1.49 | 3.2 |
| | | (5H, m), 1.82-1.87 (2H, m), 1.99-2.09 (7H, m), 2.78-2.84 | |
| | | (2H, m), 3.05-3.17 (2H, m), 3.34-3.37 (1H, m), 3.49-3.55 | |
| | | (2H, m), 4.13-4.16 (2H, m), 5.34-5.40 (1H, s), 6.0-6.1 | |
| | | (1H, br s), 7.51-7.53 (2H, d), 7.74-7.76 (2H, d), 9.33 | |
| | | (1H, s), 9.47-9.48 (1H, s), 10.04 (1H, s). | |
| I-66 | 607.0 | (d6-DMSO, 400 MHz) 1.40-1.52 (2H, m), 1.79-1.80 | 3.45 |
| | | (2H, m), 1.99 (3H, s), 2.18-2.29 (4H, m), 2.91 (2H, brs), | |
| | | 3.35-3.46 (4H, m), 3.55 (2H, q), 5.81 (1H, s), 6.87 (1H, | |
| | | s), 7.57 (2H, d), 7.70 (2H, d), 10.57 (1H, s), 10.85 (1H, s) | |
| I-67 | 603.63 | (CDCl₃) 1.44 (2H, m), 1.55 (2H, m), 1.70 (2H, m), 2.18 | 3.48 |
| | | (3H, s), 2.23 (2H, m), 2.99 (2H, t), 3.32 (6H, br m), 3.43 | |
| | | (1H, m), 3.95 (4H, m), 5.66 (1H, s), 5.92 (1H, s), 7.57 | |
| | | (2H, d), 7.74 (2H, d) | |
| I-68 | 561.4 | (d6-DMSO, 400 MHz) 1.3-1.4 (4H, m), 1.65-1.72 | 3.03 |
| | | (4H, m), 1.8-1.9 (2H, m), 2.03 (3H, s), 2.2-2.3 (1H, m), | |
| | | 2.83-2.89 (2H, m), 3.55 (2H, q), 3.9-3.98 (2H, m), 5.45 | |
| | | (1H, brs), 5.91 (1H, vbrs), 6.38 (1H, brs), 7.55 (2H, d), 7.7 | |
| | | (2H, d), 9.26 (1H, brs), 10.55 (1H, brs), 11.8 (1H, brs) | |
| I-69 | 547.0 | (d6-DMSO, 400 MHz) 1.40-1.51 (1H, m), 1.52-1.69 (3H, | 3.3 |
| | | m), 2.01 (3H, brs), 2.01-2.09 (1H, partly obscured m), | |
| | | 2.15 (3H, brs), 2.30-2.41 (1H, m), 2.06-2.68 (2H, m), | |
| | | 3.11-3.28 (2H, m), 3.53 (2H, q), 5.45 (1H, brs), 5.77 (1H, | |
| | | vbrs), .54 (2H, d), 7.67 (2H, d), 9.07 (1H, brs), 10.48 | |
| | | (1H, s), 11.65 (1H, brs). NB solvent/water peaks obscure | |
| | | some signals. | |
| I-70 | 583.22 | (d6-DMSO, 400 MHz) 2.09 (3 H, s), 2.46-2.27 (2 H, m), | 3.39 |
| | | 2.79-2.77 (6 H, m), 3.32-3.29 (1 H, m), 3.57-3.53 (2 H, | |
| | | m), 3.79-3.77 (1 H, m), 3.93-3.91 (1 H, m), 5.57 (1 H, s), | |
| | | 5.81 (1 H, br s), 7.59 (2 H, d), 7.89-7.68 (6 H, m), 9.64 | |
| | | (1 H, br s), 10.82 (1 H, s), 10.99 (1 H, br s). | |
| I-71 | 496.2 | (d6-DMSO, 400 MHz) 2.03 (3H, s), 2.2-2.3 (2H, m), 3.45- | 3.32 |
| | | 3.65 (5H, m), 5.32 (0.5H, s), 5.5 (1.5H, s), 5.85 (1H, vbrs), | |
| | | 7.58 (2H, d), 7.72 (2H, d), 9.21 (1H, s), 10.5 (1H, s), 11.65 | |
| | | (1H, s) | |
| I-72 | 577.3 | (d6-DMSO, 400 MHz) 1.25-1.35 (2H, m), 1.7-1.75 | 3.31 |
| | | (2H, m), 2.05 (3H, s), 2.2-2.3 (2H, m), 3.53-3.58 (6H, m), | |
| | | 4.02-4.05 (2H, m), 5.45 (1H, brs), 6.1 (1H, vbrs), 7.52 | |
| | | (2H, d), 7.67 (2H, d), 9.13 91H, brs), 10.45 (1, s), 11.7 | |
| | | (1H, brs) | |
| I-73 | 590.0 | (d6-DMSO, 400 MHz) 1.33-1.38 (4H, m), 1.82-1.85 (2H, | 8.52 |
| | | m), 2.10 (3H, brs), 2.25 (4H, brs), 2.81-2.87 (2H, m), | |
| | | 3.63 (2H, q), 4.10-4.15 (2H, m), 5.54 (1H, s), 6.10 (1H, | |
| | | vbrs), 7.62 (2H, d), 7.75 (2H, d), 9.23 (1H, brs), 10.56 | |
| | | (1H, s), 11.75 (1H, brs). NB solvent/water peaks obscure | |
| | | some signals | |
| I-74 | 558.3 | (d6-DMSO, 400 MHz) 2.10 (3H, s), 2.2-2.3 (2H, m), 3.45- | 3.49 |
| | | 3.65 (3H, m), 5.32 90.5H, s), 5.5 (0.5H, s), 5.55 (1H, brs), | |
| | | 5.8 (1H, vbrs), 7.6 (2H, d), 7.65-7.9 (5H, m), 9.25 (1H, s), | |
| | | 10.8 (1h, s), 11.65 (1H, s) | |
| I-75 | 571.9 | (d6-DMSO, 400 MHz) 0.8-0.86 (2H, m), 1.2-1.3 (4H, m), | 3.33 |
| | | 1.8-1.9 (2H, m), 2.0 (3H, s), 2.3-2.45 (4H, m), 2.7-2.85 | |
| | | (3H, m), 3.5-3.53 (4H, m), 4.0-4.05 (2H, m), 5.4 (1H, brs), | |
| | | 6.0 (1H, brs), 7.45 (2H, d), 7.75 (2H, d), 9.2 (1H, brs), 10.7 | |
| | | (1H, brs), 11.7 (1H, brs) | |
| I-76 | 558.3 | (d6-DMSO, 400 MHz) 2.10 (3H, s), 2.2-2.3 (2H, m), 3.45- | 3.49 |
| | | 3.65 (3H, m), 5.32 (0.5H, s), 5.5 (0.5H, s), 5.55 (1H, brs), | |
| | | 5.8 (1H, vbrs), 7.6 (2H, d), 7.65-7.9 (5H, m), 9.25 (1H, s), | |
| | | 10.8 (1h, s), 11.65 (1H, s) | |
| I-77 | 561.54 | (d6-DMSO, 400 MHz) 1.35 (2H, m), 1.62 (6H, m), 1.90 | 3.34 |
| | | (4H, m), 2.10 (6H, m), 2.40 (4H, m), 2.88 (2H, t), 3.20 | |
| | | (2H, m), 3.48 (1H, m), 3.61 (2H, m), 4.23 (2H, d), | |
| | | 5.53(1H, s), 6.20 (1H, br s), 7.55 (2H, d), 7.79 (2H, d), | |
| | | 9.34 (1H, s), 9.55 (1H, s), 10.11 (1H, s) | |
| I-78 | 496.2 | (d6-DMSO, 400 MHz) 2.03 (3H, s), 2.2-2.3 (2H, m), 3.45- | 3.32 |
| | | 3.65 (5H, m), 5.32 (0.5H, s), 5.5 (1.5H, s), 5.85 (1H, vbrs), | |
| | | 7.58 (2H, d), 7.72 (2H, d), 9.21 (1H, s), 10.5 (1H, s), 11.65 | |
| | | (1H, s) | |
| I-79 | 550.22 | (d6-DMSO, 400 MHz) 1.53 (4H, m), 2.02 (3H, s), 3.29 | 3.46 |
| | | (2H, q), 3.47 (4H, m), 3.88 (4H, s), 5.53 (1H, br s), 5.82 | |
| | | (1H, br s), 7.47 (2H, d), 7.58 (2H, d) | |
| I-80 | 508.21 | (d6-DMSO, 400 MHz) 1.29 (2H, m), 1.70 (2H, m), 2.03 | 3.17 |
| | | (3H, s), 3.04 (2H, m), 3.58 (2H, q), 3.76 (3H, m), 5.49 | |
| | | (1H, s), 6.06 (1H, br s), 7.53 (2H, d), 7.68 (2H, d), 9.27 | |
| | | (2H, d), 10.49 (1H, s) | |
| I-81 | 510.29 | (d6-DMSO, 400 MHz) 2.02 (3H, s, CH3), 2.33 (2H, m, | 3.35 |
| | | alk), 3.26-3.33 (3H, m, alk), 3.41-3.45 (H, m, alk), 3.50- | |
| | | 3.58 (3H, m, alk), 5.32 (H, brs, alk). 5.45-5.47(2H, 2xs, | |
| | | alk, ar), 5.8 (H, brs, ar), 7.51-7.54 (2H, d, ar), 7.68-7.70 | |
| | | (2JH, d, ar), 9.37 (s, NH) and 10..31 (H, s, NH) | |
| I-82 | 597.33 | (d6-DMSO, 400 MHz) 1.44 (2H, m), 2.01 (5H, br s), 2.84 | 3.59 |
| | | (2H, t), 3.56 (4H, q), 4.13 (4H, d), 5.46 (1H, s), 6.10 (1H, | |
| | | s), 7.54 (2H, d), 7.69 (2H, d), 9.26 (1H, s), 10.50(1H, s) | |
| I-83 | 533.4 | (d6-DMSO, 400 MHz) 0.10 (2H, m), 0.31 (2H, m), 0.85 | 3.01 |
| | | (1H, m), 1.24 (2H, m), 1.58 (4H, m), 1.75 (2H, d), 1.89 | |
| | | (2H, s), 2.09 (3H, m), 2.63 (6H, m), 3.84 (2H, d), | |
| | | 4.01(1H, s), 7.27 (2H, d), 7.45 (2H, d), 8.98 (1H, s), 9.79 | |
| | | (1H, s) | |
| I-84 | 528.24 | (d6-DMSO, 400 MHz) 1.96 (4H, m), 2.01 (3H, s), 3.50 | 3.61 |
| | | (6H, m), 5.46 (1H, s), 6.04 (1H, br s), 7.55 (2H, d), 7.66 | |
| | | (2H, d), 9.31 (1H, s), 10.50 (1H, s) | |
| I-85 | 510.00 | (d6-DMSO, 400 Mhz) d 1.09 (3H, t), 1.15 (9H, s), 1.25- | 3.76 |
| | | 1.35 (2H, m), 1.65-1.69 (2H, m), 2.01 (3H, s), 2.34 (2H, | |
| | | q), 3.02 (2H, t), 3.67-3.72 (1H, m), 3.78-3.81 (2H, m), | |
| | | 5.44 (1H, s), 6.04 (1H, brs), 7.47 (2H, d), 7.69 (2H, d), | |
| | | 9.17 (1H, s), 10.07 (1H, s), 11.70 (1H, s) | |

### Example 17

### Aurora-2 (Aurora A) Inhibition Assay

Compounds were screened for their ability to inhibit Aurora-2 using a standard coupled enzyme assay (Fox et al., Protein Sci., (1998) 7, 2249). Assays were carried out in a mixture of 100 mM Hepes (pH7.5), 10 mM MgCl₂, 1 mM DTT, 25 mM NaCl, 2.5 mM phosphoenolpyruvate, 300 μM NADH, 30 μg/ml pyruvate kinase and 10 μg/ml lactate dehydrogenase. Final substrate concentrations in the assay were 400 μM ATP (Sigma Chemicals) and 570 μM peptide (Kemptide, American Peptide, Sunnyvale, Calif.). Assays were carried out at 30° C. and in the presence of 40 nM Aurora-2.

An assay stock buffer solution was prepared containing all of the reagents listed above, with the exception of Aurora-2 and the test compound of interest. 55 μl of the stock solution was placed in a 96 well plate followed by addition of 2 μl of DMSO stock containing serial dilutions of the test compound (typically starting from a final concentration of 7.5 μM). The plate was preincubated for 10 minutes at 30° C. and the reaction initiated by addition of 10 μl of Aurora-2. Initial reaction rates were determined with a Molecular Devices SpectraMax Plus plate reader over a 10 minute time course. IC50 and Ki data were calculated from non-linear regression analysis using the Prism software package (GraphPad Prism version 3.0cx for Macintosh, GraphPad Software, San Diego Calif., USA).

Compounds I-2 to I-7, I-9 to I-12, I-14 to I-27, I-29 to I-85 were found to have Aurora A kinase activity at ≦10 nM Ki.

Compounds I-1, I-8, I-13, and I-28 were found to have Aurora A kinase activity at >10 nM Ki and ≦50 nM Ki.

### Example 18

### Aurora-1 (Aurora B) Inhibition Assay(radiometric)

An assay buffer solution was prepared which consisted of 25 mM HEPES (pH 7.5), 10 mM MgCl₂, 0.1% BSA and 10% glycerol. A 22 nM Aurora-B solution, also containing 1.7 mM DTT and 1.5 mM Kemptide (LRRASLG), was prepared in assay buffer. To 22 μL of the Aurora-B solution, in a 96-well plate, was added 2 μl of a compound stock solution in DMSO and the mixture allowed to equilibrate for 10 minutes at 25° C. The enzyme reaction was initiated by the addition of 16 μl stock [γ-³³P]-ATP Pi-ATP solution (˜20 nCi/μL) prepared in assay buffer, to a final assay concentration of 800 μM. The reaction was stopped after 3 hours by the addition of 16 μL 500 mM phosphoric acid and the levels of ³³P incorporation into the peptide substrate were determined by the following method.

A phosphocellulose 96-well plate (Millipore, Cat no. MAPHNOB50) was pre-treated with 100 μL of a 100 mM phosphoric acid prior to the addition of the enzyme reaction mixture (40 μL). The solution was left to soak on to the phosphocellulose membrane for 30 minutes and the plate subsequently washed four times with 200 μL of a 100 mM phosphoric acid. To each well of the dry plate was added 30 μL of Optiphase ‘SuperMix’ liquid scintillation cocktail (Perkin Elmer) prior to scintillation counting (1450 Microbeta Liquid Scintillation Counter, Wallac). Levels of non-enzyme catalyzed background radioactivity were determined by adding 16 μL of the 500 mM phosphoric acid to control wells, containing all assay components (which acts to denature the enzyme), prior to the addition of the [γ-³³P]-ATP solution. Levels of enzyme catalyzed ³³P incorporation were calculated by subtracting mean background counts from those measured at each inhibitor concentration. For each Ki determination 8 data points, typically covering the concentration range 0-10 μM compound, were obtained in duplicate (DMSO stocks were prepared from an initial compound stock of 10 mM with subsequent 1:2.5 serial dilutions). Ki values were calculated from initial rate data by non-linear regression using the Prism software package (Prism 3.0, Graphpad Software, San Diego, Calif.).

Compounds I-20, I-32, I-35, I-63, I-67, I-69 to I-72, I-74, I-76, and I-78 to I-80 were found to have Aurora B kinase activity at ≦10 nM Ki.

Compounds I-5 to I-7, I-9, I-11, I-14 to I-18, I-21, I-22, I-26, I-27, I-29, I-31, I-33, I-38 to I-40, I-42 to I-47, I-49, I-51, I-54, I-56 to I-62, I-64 to I-66, I-68, I-73, I-75, I-77, I-81, I-82, I-84 and I-85 were found to have Aurora B kinase activity >10 nM and <50 nM Ki.

Compounds I-1 to I-4, I-8, I-10, I-12, I-13, I-19, I-23 to I-25, I-28, I-30, I-34, I-36, I-37, I-41, I-48, I-50, I-52, I-53, I-55, and I-83 were found to have Aurora B kinase activity >50 nM Ki and ≦1 uM Ki.

### Example 19

### Microsomal Stability Assay

Microsomal stability was monitored by generation of depletion-time profiles in microsomes from a range of species (male CD-1 mouse, male Sprague-Dawley rat, male Beagle dog, male Cynomolgus monkey and pooled mixed gender human). Compound spiking solutions were made up by diluting down the compound stock solution in DMSO (typically 10 mM) to give a solution in acetonitrile (0.5 mM). Compound (to give final concentration of 5 μM) was incubated with a final reaction mixture (1000 μL) consisting of liver microsome protein (1 mg/mL) and a (β-nicotinamide adenine dinucleotide phosphate, reduced form (NADPH)-regenerating system (RGS) [consisting of 2 mM β-nicotinamide adenine dinucleotide phosphate (NADP), 20.5 mM isocitric acid, 0.5 U of isocitrate dehydrogenase/mL, 30 mM magnesium chloride, and 0.1 M phosphate buffer (PB) pH 7.4] in the presence of 0.1 M PB (pH 7.4).

The reaction was initiated by the addition (250 μL) of the pre-incubated RGS to the pre-incubated microsome/VRT/PB mixture (pre-incubation in both instances was for 10 minutes at 37° C.). Samples were incubated within Eppendorf vials (1.5 ml) on a heater shaker (DPC Micromix 5 (settings; form 20, amplitude 4) modified to be heated, to 37° C., by two plate heaters fixed to the deck and controlled by a Packard Manual Heater) attached to a Multiprobe II HT Ex automated liquid handler. The liquid handler was programmed (WinPREP software) to sample the microsomal incubation mixture after 0, 2, 10, 30 and 60 minutes of incubation and transfer an aliquot (100 μL) to a stop block (96-well block) containing 100 μL of chilled methanol. The % organic in the stop mixture was optimized for analysis by addition of appropriate volumes of aqueous/organic (typically 100 μL of 50:50 methanol:water).

Prior to analysis the stop block was placed on a shaker (DPC Micromix 5; 10 min, form 20, amplitude 5) to precipitate out proteins. The block was then centrifuged (Jouan GR412; 2000 rpm, 15 min, 4° C.). A sample aliquot (200 μL) was then transferred to an analysis block and the block was centrifuged again (Jouan GR412; 2000 rpm, 5 min, 4° C.) prior to being sent for analysis. Depletion profiles were determined by monitoring the disappearance of VRT by liquid chromatography-tandem mass spectrometry (LC-MS/MS). Samples were injected (20 μL; Agilent 1100 liquid chromatographic system equipped with autosampler) onto an analytical column. Mobile phase consisted of Water+0.05% (v/v) formic acid (A) and methanol+0.05% (v/v) formic acid (B).

Running a gradient method optimized for the compound of interest carried out the compound elution from analytical column. The total run time was 6 minutes with a flow rate of 0.35 mL/min. The entire column effluent entered the electrospray ionization source (positive mode) of a Micromass Quattro LC tandem mass spectrometer between 0.5 and 5.9 min of the run. The mass spectrometry was optimized for the compound of interest. All incubations were conducted in duplicate and results were expressed as % parent remaining at either 30 minutes or 60 minutes relative to 0 minutes sample.

The following compounds were found to have >50% parent remaining after 30 minutes incubation with human liver microsomes: I-2, I-11, I-16, I-18 to I-20, I-32, I-34, I-35, I-40, I-43, I-47 to I-50, I-53 to I-57, I-60, I-62 to I-65, I-67, I-70 to I-78, I-80, I-81, and I-83.

The following compounds were found to have >50% parent remaining after 60 minutes incubation with human liver microsomes: I-7, I-11, I-18 to I-20, I-26, I-31 to I-35, I-41, I-47, I-49, I-51, I-53, I-54, I-56, I-57, I-59, I-65, I-68, I-71, I-73, I-74, I-76 to I-78, I-81, and I-83.

### Example 20

### Analysis of Cell Proliferation

Compounds were screened for their ability to inhibit cell proliferation using Colo205 cells obtained from ECACC and using the assay shown below.

Colo205 cells were seeded in 96 well plates and serially diluted compound was added to the wells in duplicate. Control groups included untreated cells, the compound diluent (0.1% DMSO alone) and culture medium without cells. The cells were then incubated for 72 or 96 hrs at 37C in an atmosphere of 5% CO2/95% humidity.

To measure proliferation, 3 h prior to the end of the experiment 0.5 μCi of 3H thymidine was added to each well. Cells were then harvested and the incorporated radioactivity counted on a Wallac microplate beta-counter. Dose response curves were calculated using either Prism 3.0 (GraphPad) or SoftMax Pro 4.3.1 LS (Molecular Devices) software.

The following compounds had IC50 values of ≦25 nM after 72 hours: I-56, I-59, and I-63 to I-74.

The following compounds had IC50 values of >25 nM and ≦125 nM after 72 hours: I-26, I-27, I-41, I-53, and I-75 to I-84.

The following compounds had IC50 values of ≦50 nM after 96 hours: I-36 to I-62.

The following compounds had IC50 values of >50 nM and ≦200 nM after 96 hours: I-8 to I-35.

The following compounds had IC50 values of >200 nM and ≦1 uM after 96 hours: I-1 to I-7 and I-85.

### Example 21

### Abl Kinase Activity Inhibition Assay and Determination of the Inhibition Constant Ki

Compounds were screened for their ability to inhibit N-terminally truncated (Δ 27) Abl kinase activity using a standard coupled enzyme system (Fox et al., Protein Sci., 7, pp. 2249 (1998)). Reactions were carried out in a solution containing 100 mM HEPES (pH 7.5), 10 mM MgCl₂, 25 mM NaCl, 300 μM NADH, 1 mM DTT and 3% DMSO. Final substrate concentrations in the assay were 110 μM ATP (Sigma Chemicals, St Louis, Mo.) and 70 μM peptide (EAIYAAPFAKKK, American Peptide, Sunnyvale, Calif.). Reactions were carried out at 30° C. and 21 nM Abl kinase. Final concentrations of the components of the coupled enzyme system were 2.5 mM phosphoenolpyruvate, 200 μM NADH, 60 μg/ml pyruvate kinase and 20 μg/ml lactate dehydrogenase.

An assay stock buffer solution was prepared containing all of the reagents listed above with the exception of ATP and the test compound of interest. The assay stock buffer solution (60 μl) was incubated in a 96 well plate with 2 μl of the test compound of interest at final concentrations typically spanning 0.002 μM to 30 μM at 30° C. for 10 min. Typically, a 12 point titration was prepared by serial dilutions (from 1 mM compound stocks) with DMSO of the test compounds in daughter plates. The reaction was initiated by the addition of 5 μl of ATP (final concentration 110 μM). Rates of reaction were obtained using a Molecular Devices Spectramax plate reader (Sunnyvale, Calif.) over 10 min at 30° C. The Ki values were determined from the residual rate data as a function of inhibitor concentration using nonlinear regression (Prism 3.0, Graphpad Software, San Diego, Calif.).

Compounds I-4, I-18, I-24, I-29, I-38, I-39, I-46, I-52, I-53, I-57, I-60, I-68, and I-78 were found to inhibit Abl kinase at a Ki value of <25 nM.

Compounds I-7, I-12, I-27, I-41, I-42, I-63, I-65, I-69, I-71, I-80, and I-81 were found to inhibit Abl kinase at a Ki value of ≧25 nM and ≦100 nM.

### Example 22

### Mutant Abl Kinase (T315I) Activity Inhibition Assay and Determination of the Inhibition Constant IC50

Compounds were screened for their ability to inhibit the T315I mutant form of human Abl at Upstate Cell Signaling Solutions (Dundee, UK). In a final reaction volume of 25 μl, the T315I mutant of human Abl (5-10 mU) was incubated with 8 mM MOPS pH 7.0, 0.2 mM EDTA, 50 μM EAIYAAPFAKKK, 10 mM Mg Acetate, [γ-³³P-ATP] (specific activity approx. 500 cpm/pmol, 10 mM final assay concentration) and the test compound of interest at final concentrations over the range 0-4 μnM. The reaction was initiated by the addition of the MgATP mix. After incubation for 40 minutes at room temperature, the reaction was stopped by the addition of 5 μl of a 3% phosphoric acid solution. 10 μl of the reaction was then spotted onto a P30 filtermat and washed three times for 5 minutes in 75 mM phosphoric acid and once in methanol prior to drying and scintillation counting. Inhibition IC50 values were determined from non-linear regression analysis of the residual enzyme activities as a function of inhibitor concentration (Prism 3.0, Graphpad Software, San Diego, Calif.).

Compounds I-7, I-27, I-29, I-41, I-53, I-63, I-65, I-68, I-69, I-71, I-72, I-78, I-80, and I-81 were found to inhibit Mutant Abl Kinase (T315I) kinase at a Ki value of <200 nM.

Compounds I-18, I-42, I-55, I-61, and I-82 were found to inhibit Mutant Abl Kinase (T315I) kinase at a Ki value of ≧200 nM and ≦500 nM.

While we have described a number of embodiments of this invention, it is apparent that our basic examples may be altered to provide other embodiments that utilize or encompass the compounds, methods, and processes of this invention. Therefore, it will be appreciated that the scope of this invention is to be defined by the appended claims.

We claim:

1. A compound of formula I: 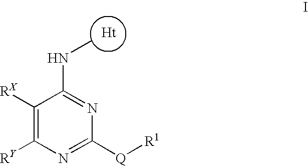
or a pharmaceutically acceptable salt thereof, wherein:
Ht is 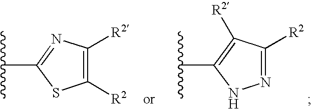
R² is H, C₁₋₃ alkyl, or cyclopropyl;
R^{2′} is H;
Q is —O—, —S—, or —C(R′)₂—;
R^{X} is H or F;
R^{Y} is 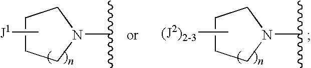
J¹ is F, NR⁴R⁵, CN, OR⁶, oxo (═O), or C₂₋₆alkyl optionally substituted with 1 occurrence of OH or OCH₃;
each J² is independently C₁₋₆alkyl, F, NR⁴R⁵, CN, or OR⁶; or two J² groups, together with the atom(s) to which they are bound, form a 4-7 membered heterocyclyl ring containing 1-2 heteroatoms selected from N or O; wherein said ring is optionally substituted with 0-3 J^{R};
n is 1 or 2;
R⁴ is H, C₁₋₅alkyl, or C₃₋₆ cycloalkyl;
R⁵ is C₁₋₅alkyl or C₃₋₆ cycloalkyl;
or R⁴ and R⁵, together with the nitrogen atom to which they are bound, form a 3-6 membered monocyclic ring containing 1-2 heteroatoms selected from O, N, or S; wherein said monocyclic ring is optionally substituted with 0-3 J^{R};
R⁶ is H, C₁₋₄alkyl or C₃₋₆ cycloalkyl; wherein said C₁₋₄alkyl or C₃₋₆ cycloalkyl is optionally substituted with 1-3 fluorine atoms;
J^{R} is F or R⁷;
R¹ is phenyl or a 6-membered heteroaryl ring, wherein said heteroaryl has 1-4 ring heteroatoms selected from O, N, and S; R¹ is optionally substituted with 0-4 occurrences of —NHC(O)R³ or 0-4 fluorine atoms;
R³ is C₁₋₆aliphatic or phenyl, wherein said R³ is optionally substituted with 0-6 J³;
each J³ is independently halo, C₁₋₆alkyl, —O—(C₁₋₆alkyl), —S—(C₁₋₆ alkyl), nitro, or CN, wherein said C₁₋₆alkyl group is optionally substituted with 0-3 flourine atoms; or two J³ groups, together with the carbon atom to which they are bound, form a 3-5 membered monocyclic group containing 0-1 heteroatom selected from O, N, and S;
each R⁷ is independently C₁₋₆ aliphatic; a 5-6 membered heteroaryl containing 1-4 heteroatoms selected from O, N, or S; each R⁷ is optionally substituted with 0-3 J⁷; and
J⁷ is independently NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄aliphatic), or haloC₁₋₄aliphatic.

2. The compound of claim 1, wherein Q is S.

3. The compound of claim 1 or claim 2, wherein R^{X} is H.

4. The compound of any one of claims 1-3, wherein R² is C₁₋₃alkyl or cyclopropyl.

5. The compound of claim 4, wherein R² is C₁₋₃ alkyl.

6. The compound of any one of claims 1-5, wherein R^{2′} is H.

7. The compound of any one of claims 1-6, wherein R¹ is phenyl.

8. The compound of claim 7, wherein Ht is

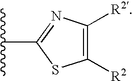

9. The compound of claim 7, wherein Ht is

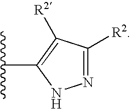

10. The compound of claim 8 or claim 9, wherein n is 1.

11. The compound of claim 8 or claim 9, wherein n is 2.

12. The compound of claim 10 or claim 11, wherein each J² is independently C₁₋₆alkyl, F, NR⁴R⁵, CN, or OR⁶.

13. The compound of claim 10 or claim 11, wherein two J² groups, together with the atom(s) to which they are bound, form a 4-7 membered heterocyclyl ring containing 1-2 heteroatoms selected from N or O; wherein said ring is optionally substituted with 0-3 J^{R}.

14. The compound of claim 12 or claim 13, wherein R¹ is substituted at the para position.

15. The compound of claim 14, wherein R¹ is optionally substituted with 1 occurrence of —NHC(O)R³.

16. The compound of claim 15, wherein R³ is C₁₋₆aliphatic wherein said R³ is optionally substituted with 0-6 J³.

17. The compound of claim 16, wherein R³ is —CH₂CH₃, —CH₂CF₃, —CH₂CH₂CF₃, cyclopropyl, or

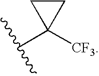

18. The compound of claim 15, wherein R³ is phenyl.

19. The compound of claim 18, wherein R³ is substituted in the ortho position with J³.

20. The compound of claim 19, wherein J³ is halogen, —CF₃, C₁₋₃alkyl, —S—(C₁₋₃alkyl), or OCF₃.

21. The compound of any one of claims 1-9, 14-20, wherein
R^{Y} is 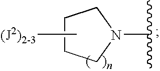
n is 2; and
two J² groups, together with the atom(s) to which they are bound, form a 4-7 membered heterocyclyl ring containing 1-2 heteroatoms selected from N or O; wherein said ring is optionally substituted with 0-3 J^{R}.

22. The compound of claim 21, wherein the two J² groups, together with the atom to which they are bound, form a 4-7 membered spirocyclic heterocyclyl ring containing 1-2 heteroatoms selected from N or O; wherein said ring is optionally substituted with 0-3 J^{R}.

23. The compound of claim 21, wherein the two J² groups together with the atom(s) to which they are bound, form a 5-membered spirocyclic heterocyclyl ring containing 1 heteroatom selected from N or O; wherein said ring is optionally substituted with 0-3 J^{R}.

24. The compound of claim 23, wherein the two J² groups together with the atom(s) to which they are bound, form a 5-membered spirocyclic heterocyclyl ring containing 1 N heteratom; wherein said ring is optionally substituted with 0-3 J^{R}.

25. The compound of claim 24, wherein the two J² groups together with the atom(s) to which they are bound, form a 5-membered spirocyclic heterocyclyl ring containing 1 N heteratom; wherein said ring is optionally substituted with 1 J^{R}.

26. The compound of claim 25, wherein the J^{R} is R⁷ and the R⁷ is C₁₋₆alkyl.

27. The compound of claim 26, wherein R¹ is phenyl and R¹ is substituted with 0-4 occurrences of —NHC(O)R³.

28. The compound of claim 27, wherein R³ is C₁₋₆alkyl.

29. The compound of claim 27, wherein R³ is cyclopropyl.

30. The compound of claim 25, wherein R¹ is

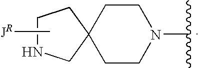

31. The compound of claim 30, wherein R^{Y} is

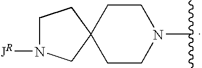

32. The compound of claim 31, wherein J^{R} is CH₃.

33. The compound of any one of claims 30-32, wherein R¹ is

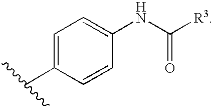

34. The compound of claim 33, wherein R³ is C₁₋₆aliphatic.

35. The compound of claim 34, wherein R³ is ethyl or cyclopropyl.

36. The compound of any one of claims 1-9, wherein
R^{Y} is 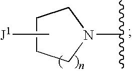
n is 1;
J¹ is F;
R¹ is substituted with 1 occurrence of —NHC(O)R³;
R³ is C₁₋₆aliphatic, wherein said R³ is substituted with 0-6 J³;
each J³ is halo.

37. The compound according to claim 36, wherein R^{Y} is

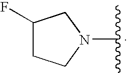

38. The compound according to claim 37, wherein R^{Y} is

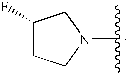

39. The compound according to any one of claims 36-38, wherein R³ is CH₂CF₃ or CH₂CH₂CF₃.

40. The compound according to any one of claims 36-38, wherein R³ is ethyl or cyclopropyl.

41. The compound according to claim 39, wherein R³ is CH₂CF₃.

42. The compound of any one of claims 1-9, wherein
R¹ is 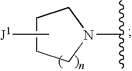
n is 1;
J¹ is NR⁴R⁵;
R¹ is substituted with 1 occurrence of —NHC(O)R³;
R³ is C₁₋₆aliphatic, wherein said R³ is substituted with 0-6 J³;
each J³ is halo.

43. The compound according to claim 41, wherein R^{Y} is

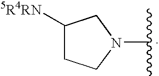

44. The compound according to claim 42, wherein R^{Y} is

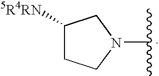

45. The compound according to any one of claims 42-44, wherein R³ is CH₂CF₃ or CH₂CH₂CF₃.

46. The compound according to any one of claims 42-44, wherein R³ is ethyl or cyclopropyl.

47. The compound according to claim 45, wherein R³ is CH₂CF₃.

48. The compound of claim 1 wherein the compounds are selected from the following:

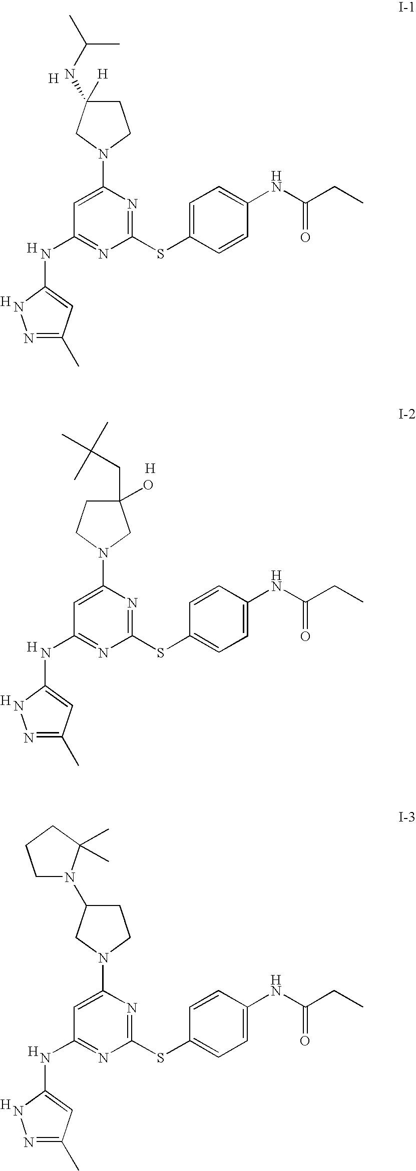

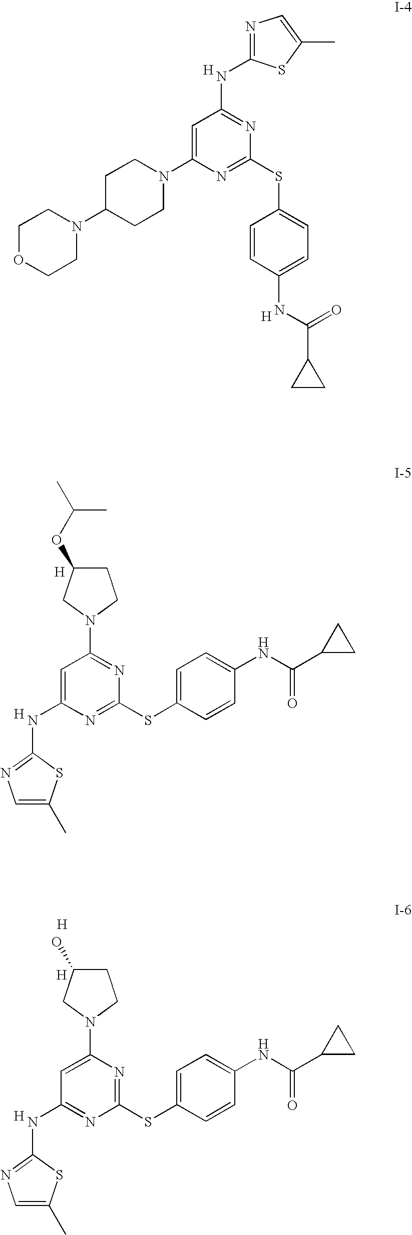

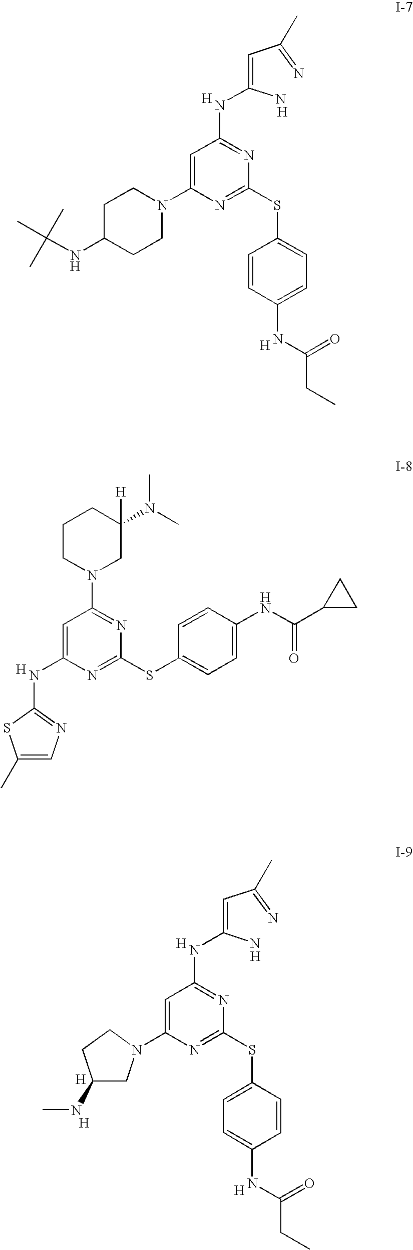

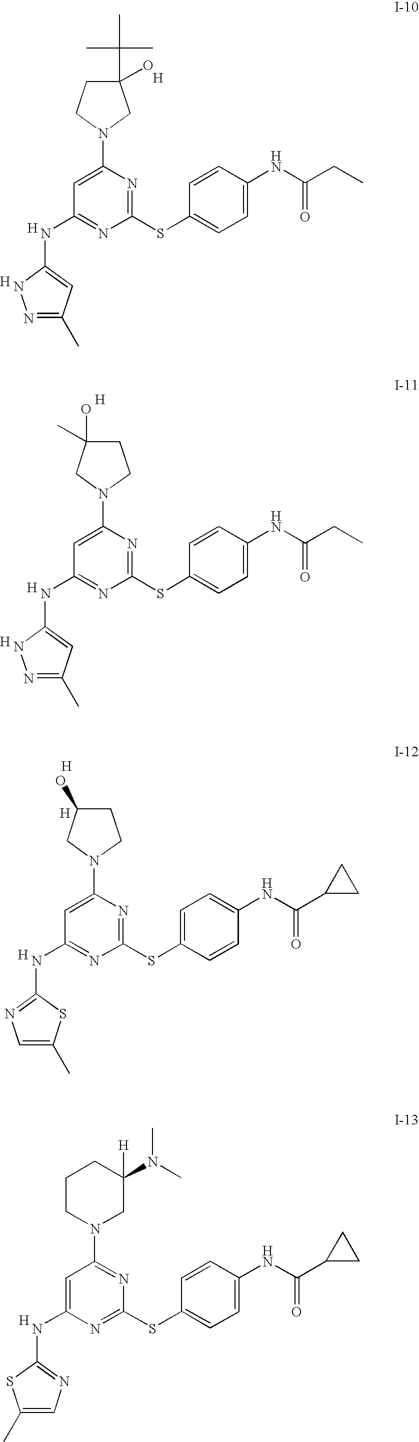

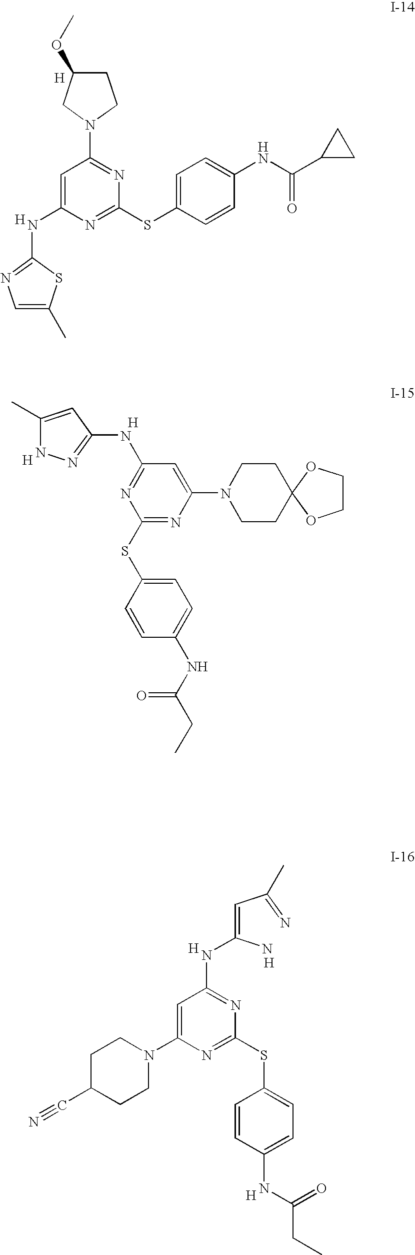

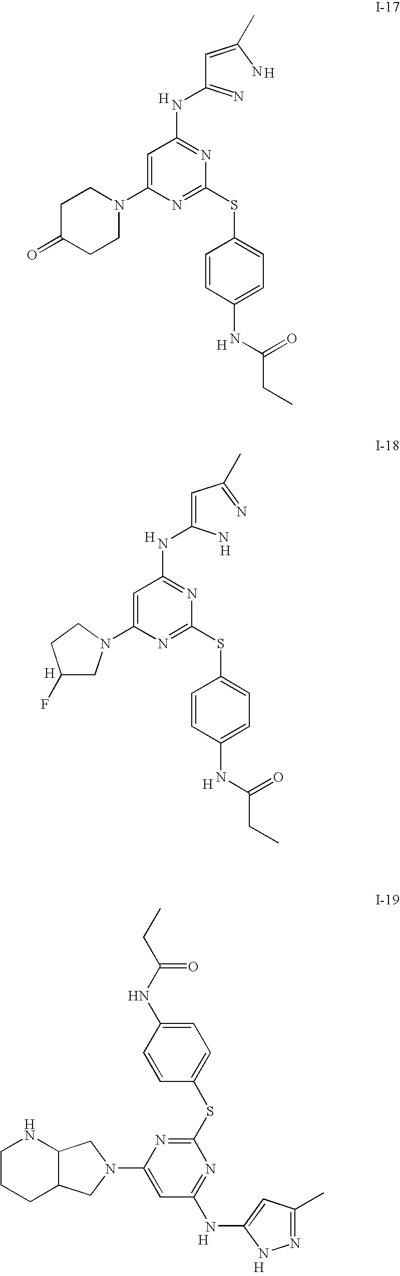

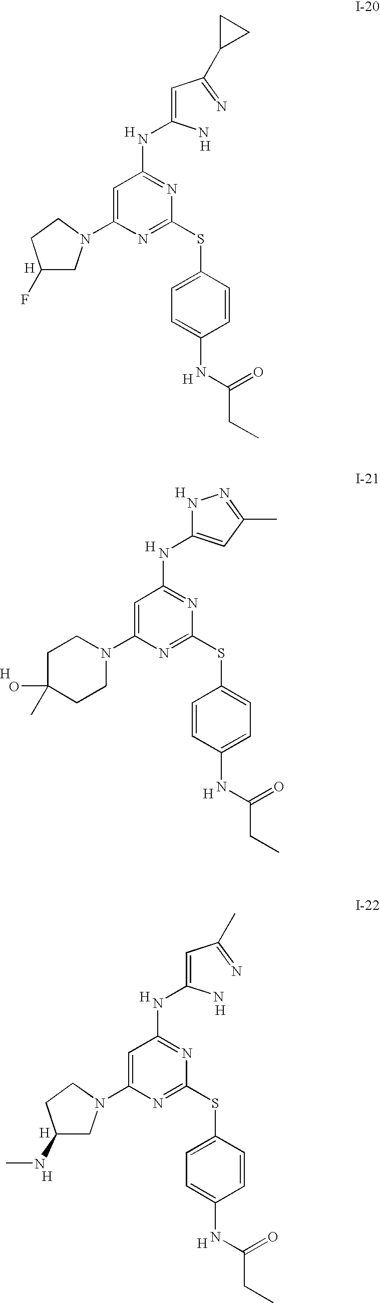

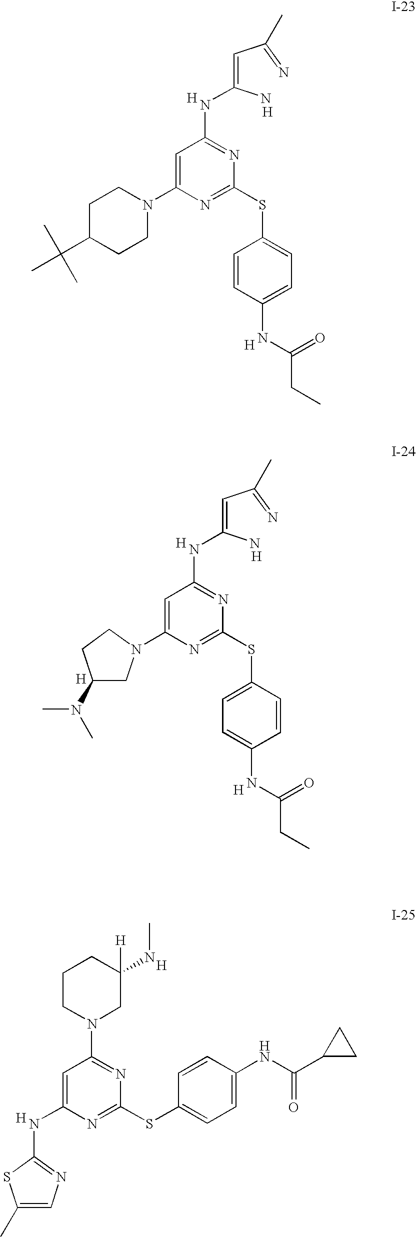

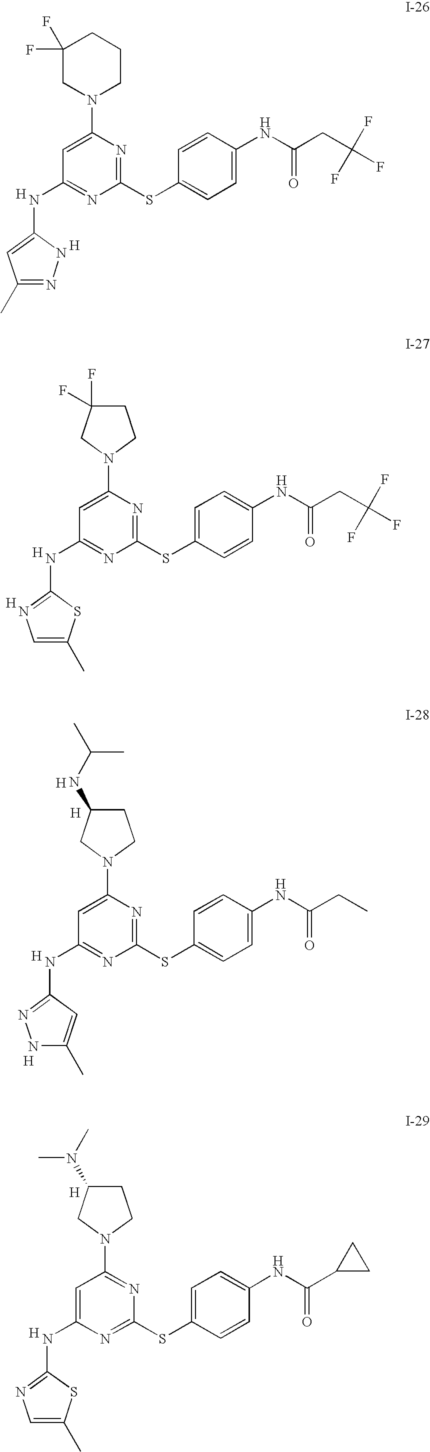

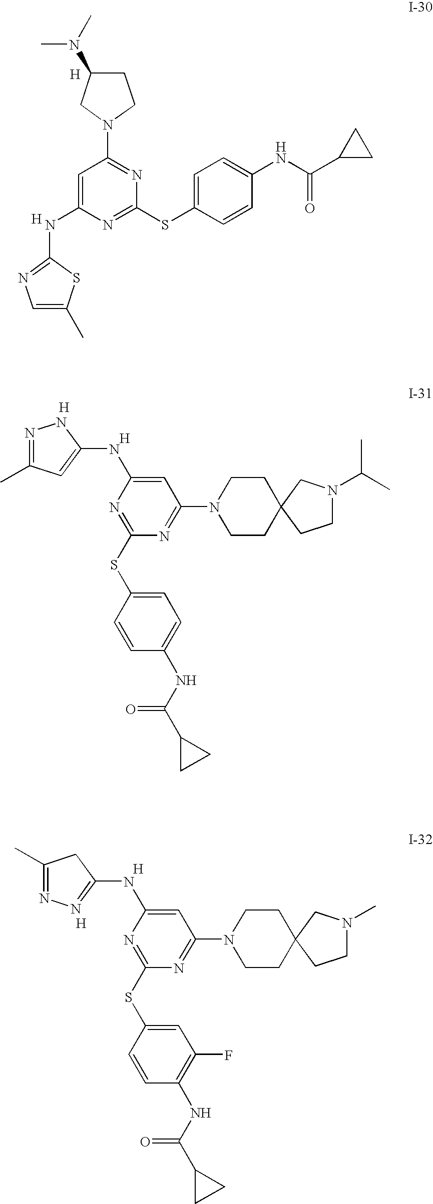

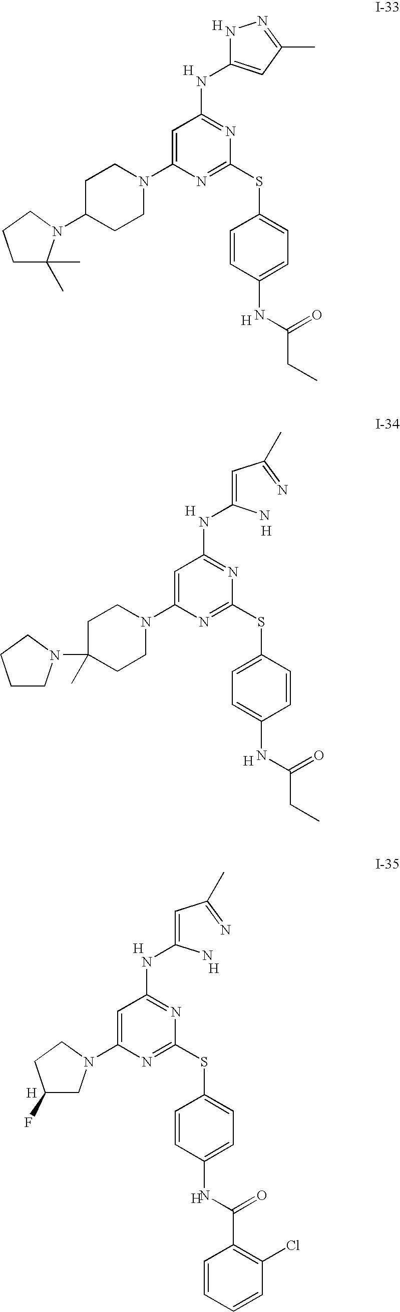

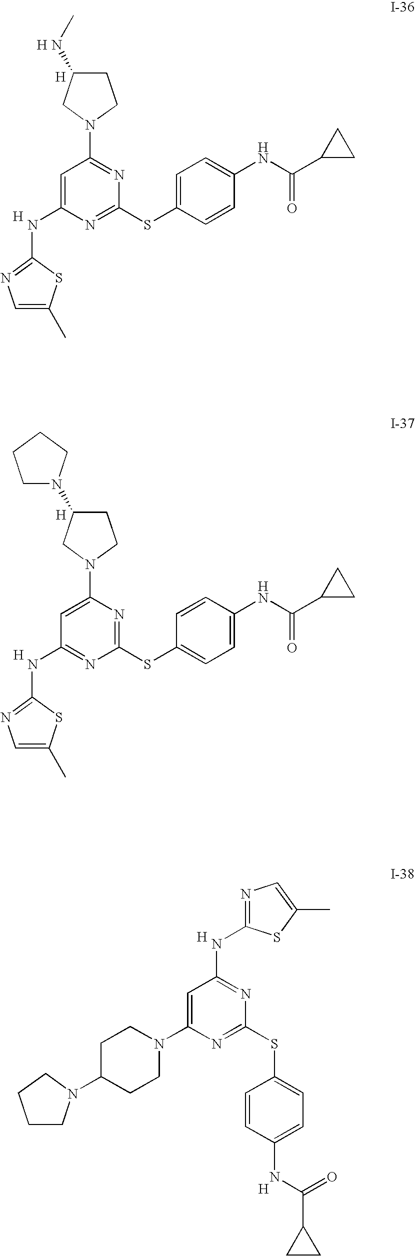

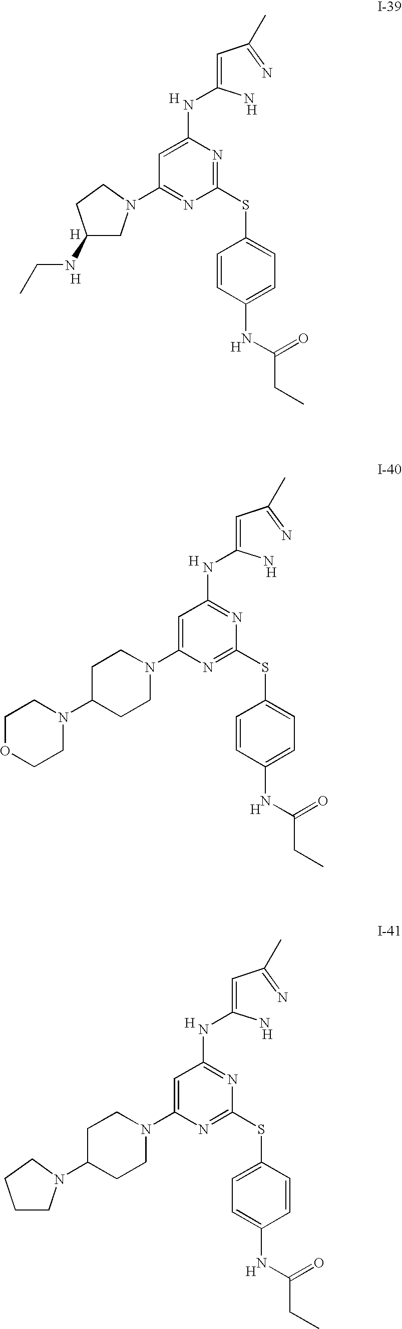

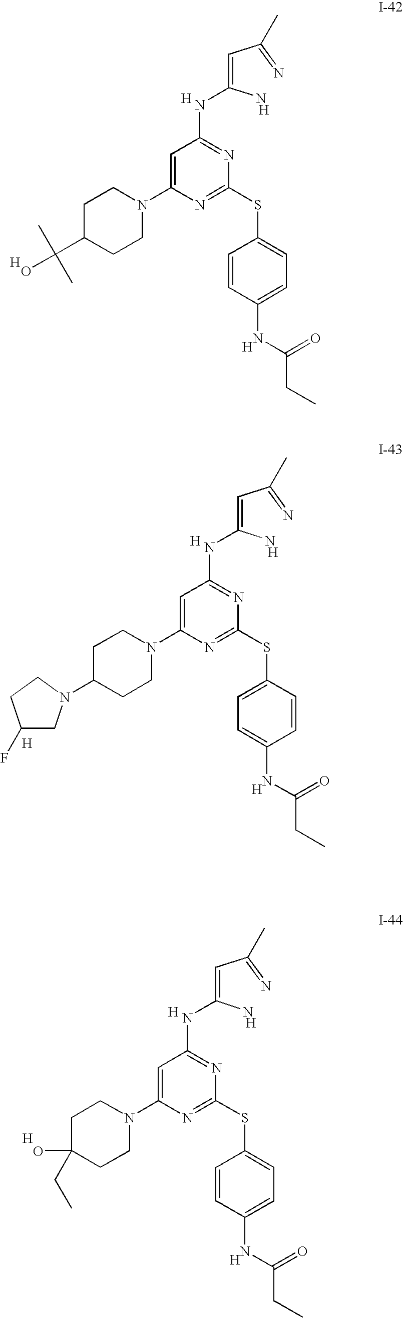

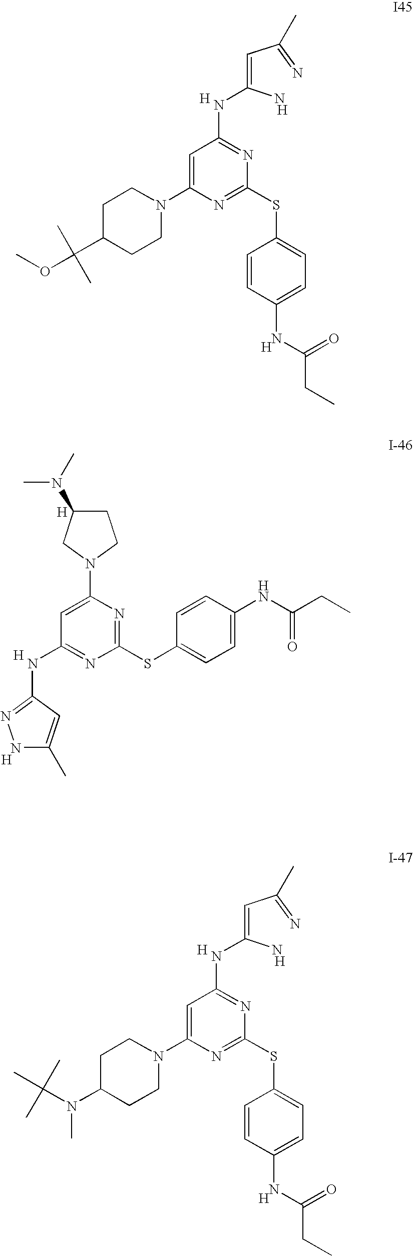

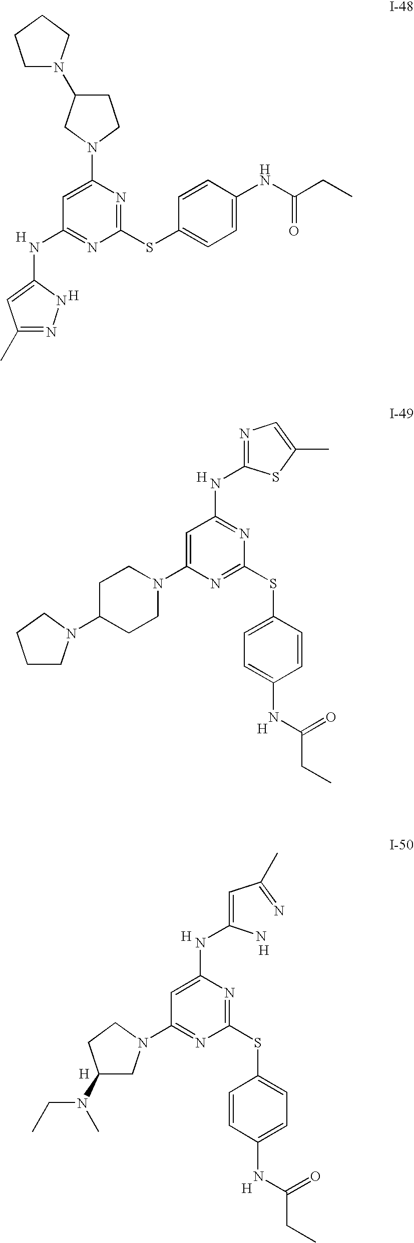

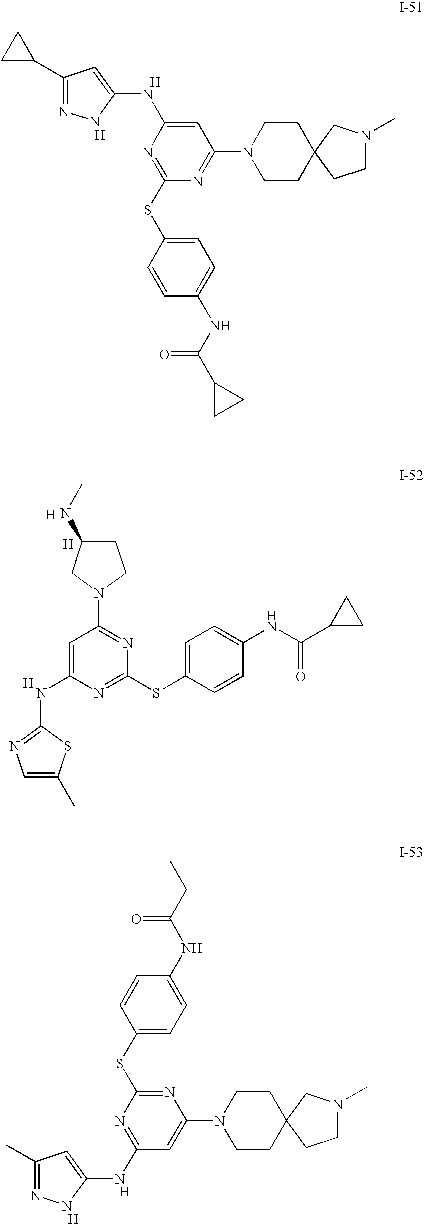

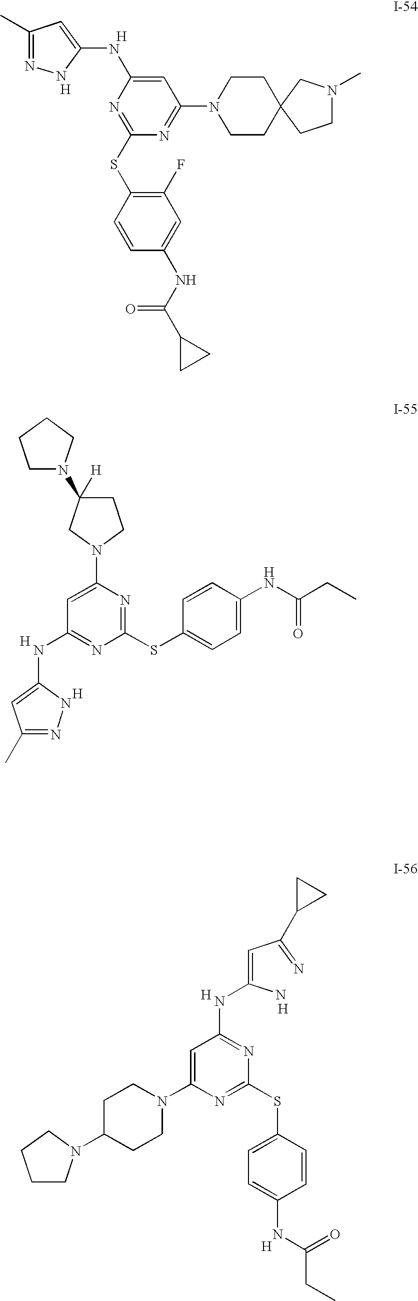

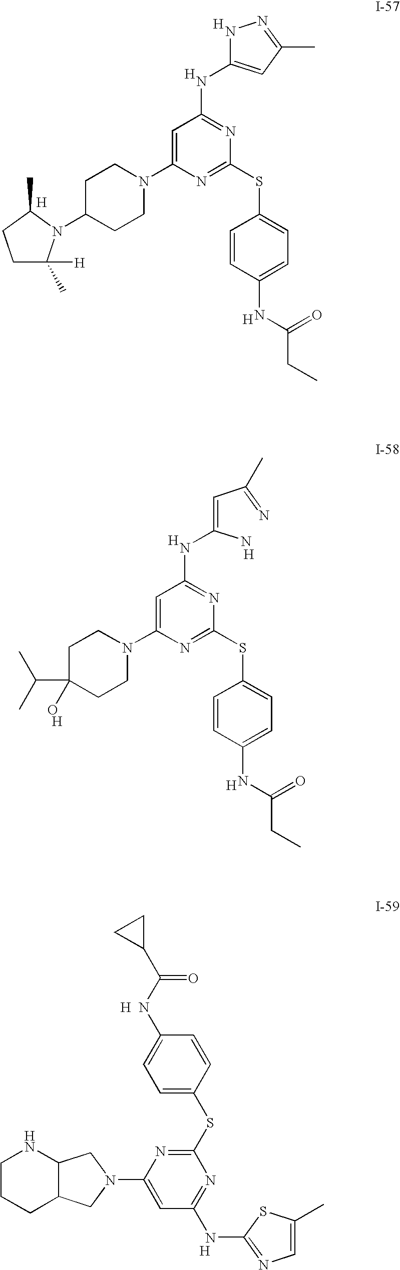

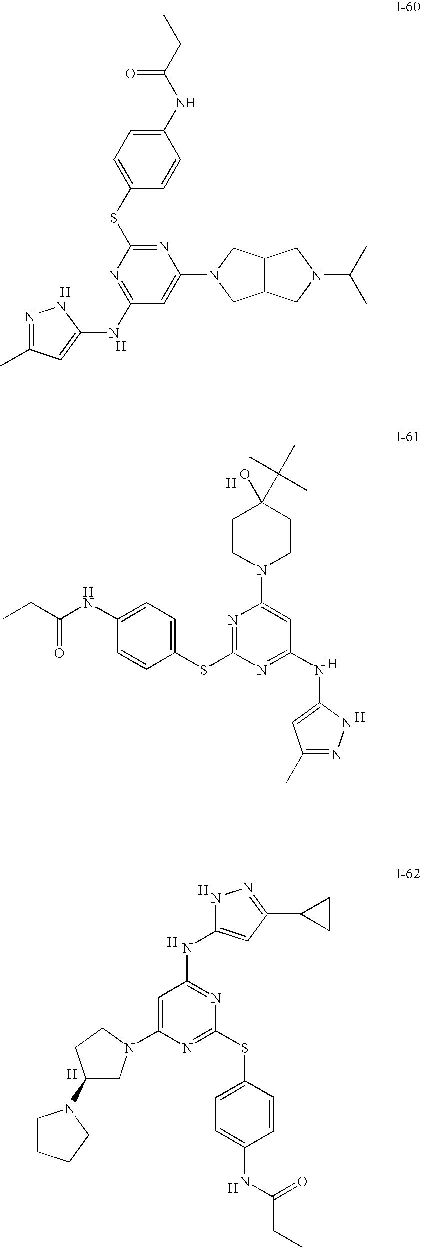

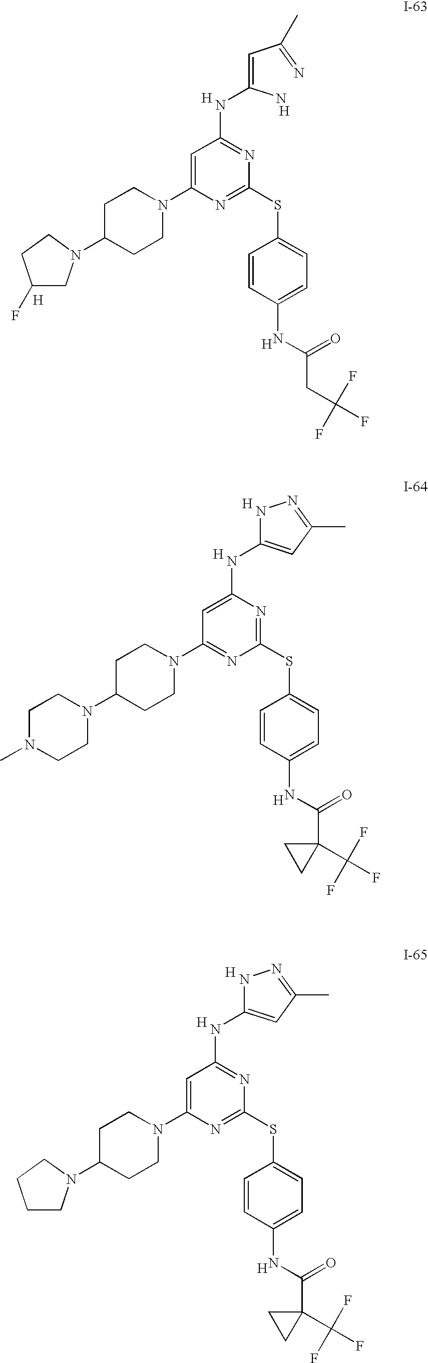

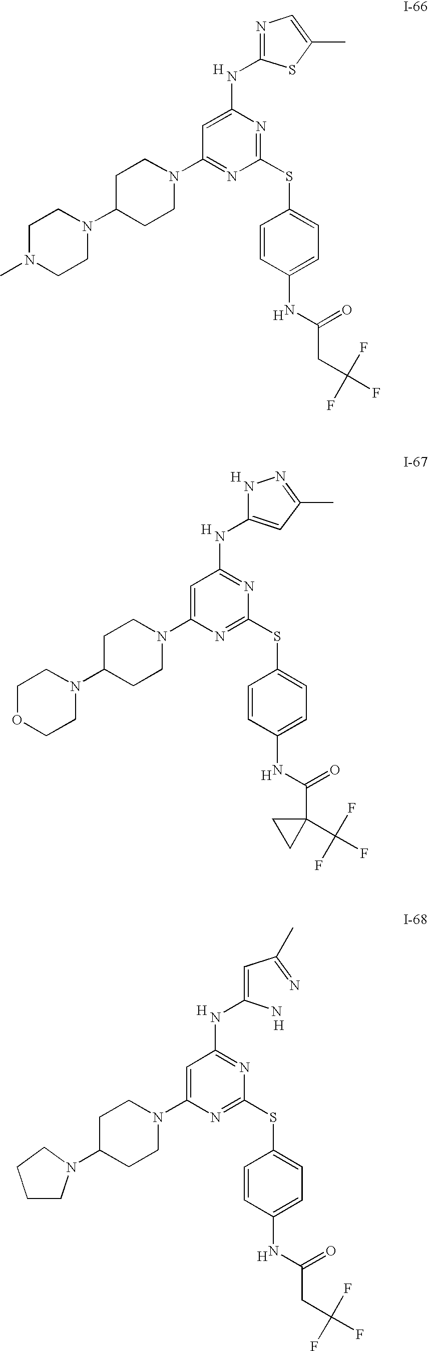

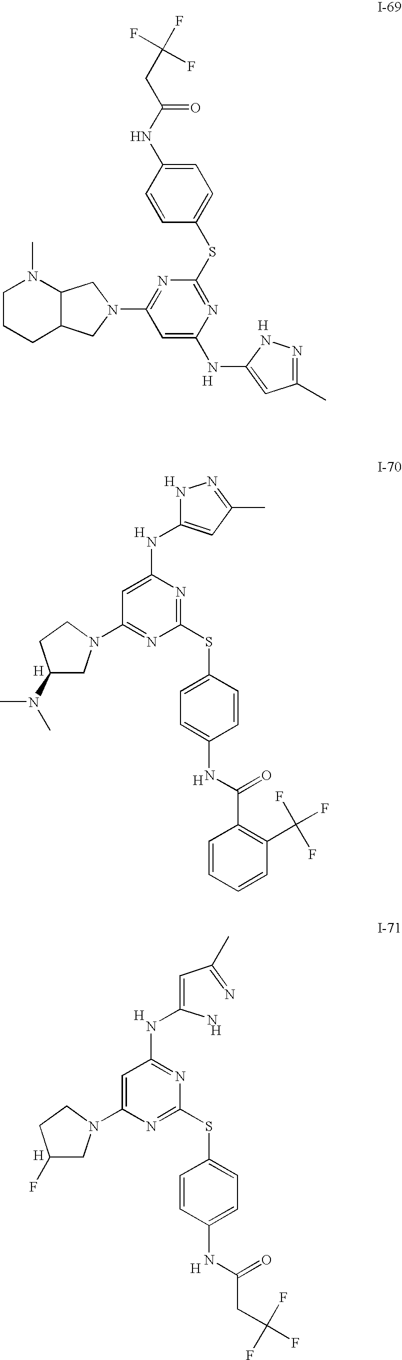

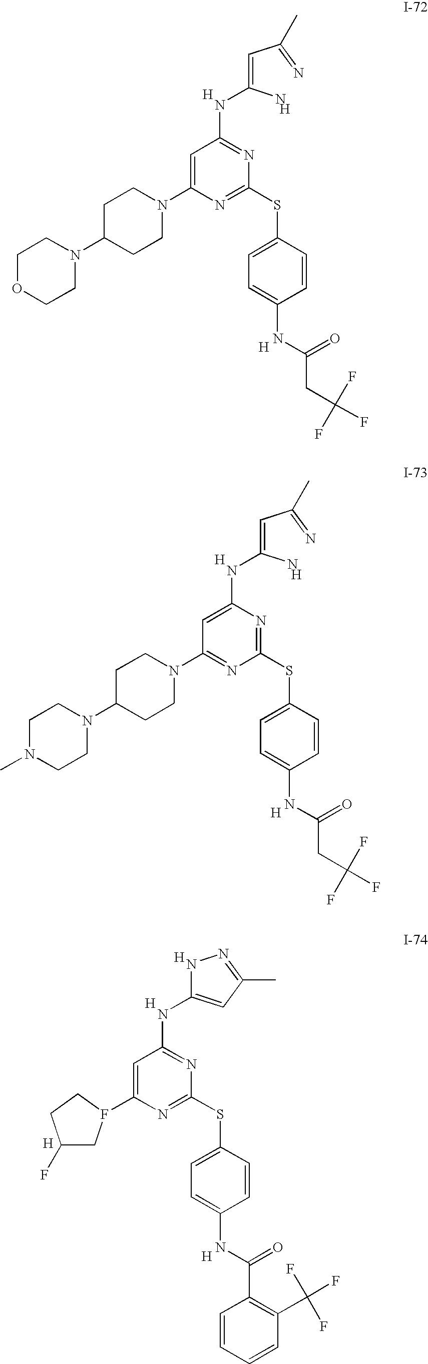

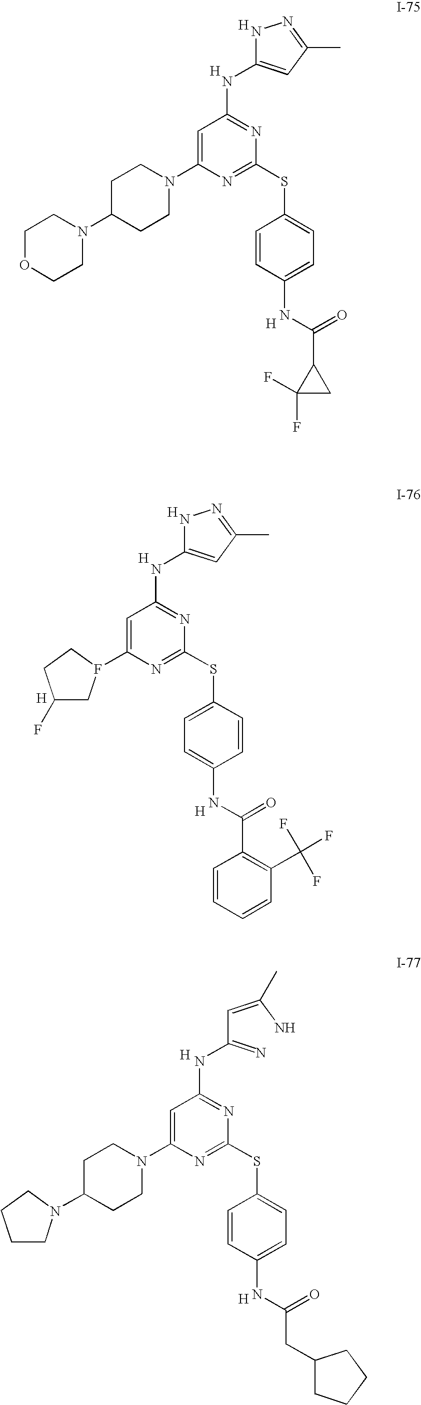

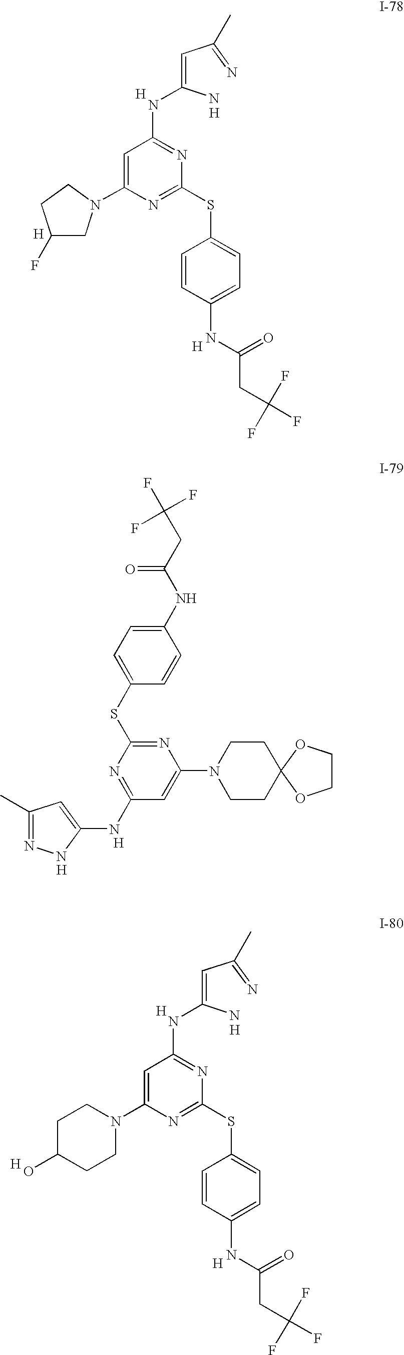

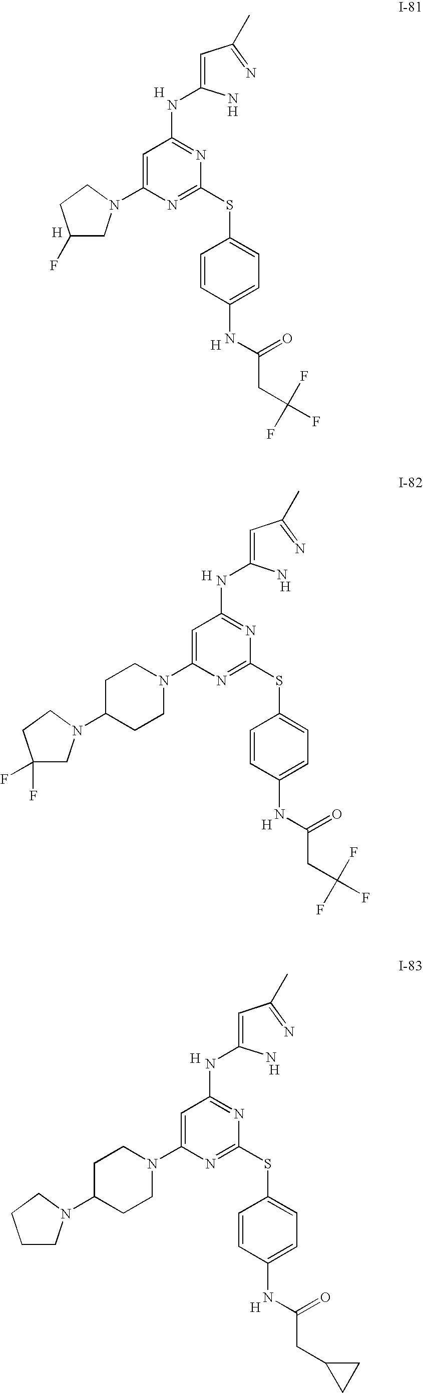

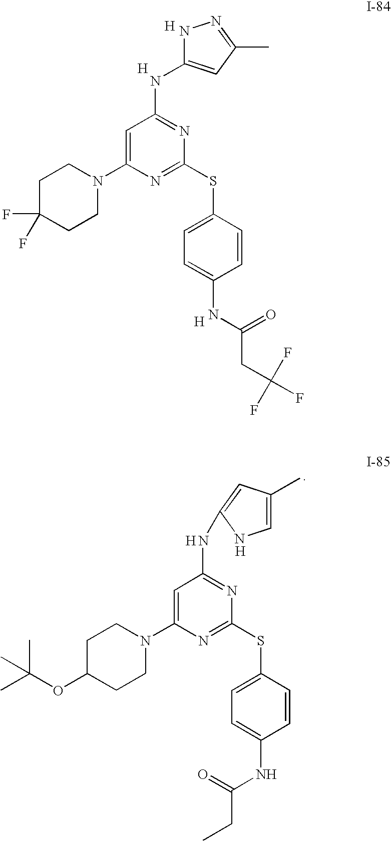

49. A composition comprising a compound of formula I: 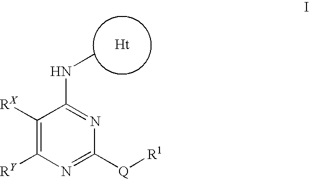
or a pharmaceutically acceptable salt thereof, wherein the variables are as defined according to any one of claims 1-48.

50. A method of inhibiting Aurora protein kinase activity in a biological sample comprising contacting said biological sample with a compound of formula I: 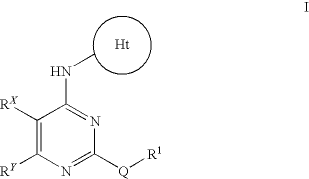
or a pharmaceutically acceptable salt thereof, wherein the variables are as defined according to any one of claims 1-48.

51. A method of treating a proliferative disorder in a patient comprising the step of administering to said patient a compound of formula I: 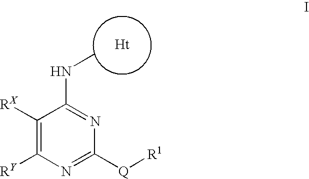
or a pharmaceutically acceptable salt thereof, wherein the variables are as defined according to any one of claims 1-48.

52. The method according to claim 49, wherein said proliferative disorder is cancer.

53. The method according to claim 49, wherein said proliferative disorder is selected from melanoma, myeloma, leukemia, lymphoma, neuroblastoma, or a cancer selected from colon, breast, gastric, ovarian, cervical, lung, central nervous system (CNS), renal, prostate, bladder, pancreatic, brain (gliomas), head and neck, kidney, liver, melanoma, sarcoma, or thyroid cancer.

54. The method according to claim 51-53 further comprising the sequential or co-administration of a therapeutic agent.

55. The method according to claim 54, wherein said therapeutic agent is selected from taxanes, inhibitors of bcr-abl, inhibitors of EGFR, DNA damaging agents, and antimetabolites.

56. The method according to claim 54, wherein said therapeutic agent is selected from Paclitaxel, Gleevec, dasatinib, nilotinib, Tarceva, Iressa, cisplatin, oxaliplatin, carboplatin, anthracyclines, AraC and 5-FU.

57. The method according to claim 54, wherein said therapeutic agent is selected from camptothecin, doxorubicin, idarubicin, Cisplatin, taxol, taxotere, vincristine, tarceva, the MEK inhibitor, U0126, a KSP inhibitor, vorinostat, Gleevec, dasatinib, and nilotinib.

58. The method according to claim 54, wherein said therapeutic agent is dasatinib.

59. The method according to claim 54, wherein said therapeutic agent is nilotinib.
